(19) United States
(12) Patent Application Publication
(10) Pub. No.: US 2025/0366837 A1
(43) Pub. Date: Dec. 4, 2025

(54) Disposable Cryobiopsy System

(71) Applicant: Serpex Medical, Inc., Santa Clara, CA (US)

(72) Inventors: Ryan Bondy, Newport Beach, CA (US); Glen Rabito, Lake Forest, CA (US); Mitch McBride, Somerville, MA (US); Jack Sattell, Cambridge, MA (US); Robert C. Taft, Orange, CA (US); Alexander Siegel, Aliso Viejo, CA (US)

(73) Assignee: Serpex Medical, Inc., Santa Clara, CA (US)

(21) Appl. No.: 19/224,408

(22) Filed: May 30, 2025

Related U.S. Application Data
(60) Provisional application No. 63/654,828, filed on May 31, 2024.

(51) Int. Cl. A61B 10/04; A61B 10/02; A61B 18/02

(52) U.S. Cl. CPC A61B 10/04; A61B 10/0233; A61B 18/02; A61B 2010/0208; A61B 2018/0293

(57) ABSTRACT

A cryobiopsy device may be comprised of a sampling system forming a handle for a user to hold and a refrigerant system providing a refrigerant to the sampling system from a removable gas canister. The sampling system also includes a refrigerant controller connected to a valve assembly for controlling the flow of refrigerant from the refrigerant system, and a probe assembly cooled by the refrigerant to retrieve tissue samples via cryo-adhesion. The sampling system may further include a position controller to extend and retract a distal tip of the probe assembly based on a desired sample size.

### RELATED APPLICATIONS

This application claims the benefit of and priority to U.S. Provisional Application Ser. No. 63/654,828, filed May 31, 2024, and entitled DEVICE FOR CRYOBIOPSY, which is hereby incorporated herein by reference in its entirety.

### BACKGROUND OF THE INVENTION

Endoscopic procedures have revolutionized the field of minimally invasive medicine, enabling clinicians to diagnose and treat various conditions with reduced patient trauma and faster recovery times. A critical aspect of many endoscopic interventions is the collection of tissue samples for histopathological analysis, particularly in the diagnosis of cancer, inflammatory diseases, and other pathological conditions. Traditional methods for tissue collection, such as forceps biopsy, suction-based retrieval, and needle aspiration, often present challenges including mechanical trauma, inadequate sample size, and difficulty in securing fragile or mobile tissues.

Cryo-adhesion technology has emerged as a promising alternative for atraumatic tissue manipulation and collection. By utilizing localized cooling, cryo-adhesion probes can temporarily adhere to biological tissues, allowing for controlled extraction without, for example, imparting the mechanical forces which often lead to tissue crush damage in traditional forceps biopsies. Further, cryo-adhesion devices may help to improve sample integrity and procedural efficiency, and additionally enable the collection of larger samples, as tissue may adhere to an entire surface area of a cryo-probe, rather than, for example, being retained only within the small space between a pair of pivotable forceps jaws.

### SUMMARY OF THE INVENTION

In some aspects, the techniques described herein relate to a cryobiopsy device, including: a refrigerant system including a refrigerant cartridge; and a sampling system including: a distal tip translatable relative to an outer sheath; a position controller connected to the distal tip; and, a gas controller.

In some aspects, the techniques described herein relate to a cryobiopsy device, wherein the position controller is translatable between at least a first position Zin which the distal tip is within an outer sheath and a second position in which the distal tip extends distally beyond the outer sheath; and, wherein the sampling system further includes a refrigerant controller movable between an open position in which refrigerant may flow through the sampling system to the distal tip and a closed position in which refrigerant is prevented from flowing through the sampling system to the distal tip.

In some aspects, the techniques described herein relate to a cryobiopsy device, wherein the refrigerant system further includes: a cartridge nest holding the refrigerant cartridge; and a base housing receiving the cartridge nest and fluidly connecting the refrigerant cartridge to sampling system.

In some aspects, the techniques described herein relate to a cryobiopsy device, wherein the base housing defines a first plurality of threads and the cartridge nest defines a second plurality of threads engaged with the first plurality of threads.

In some aspects, the techniques described herein relate to a cryobiopsy device, wherein the base housing includes a piercing element passing through a septum of a refrigerant cartridge within the cartridge nest.

In some aspects, the techniques described herein relate to a cryobiopsy device, further including a valve assembly in fluid communication with the refrigerant system, the valve assembly movable between an open position in which gas flows through the valve assembly to the distal tip and a closed position in which refrigerant is prevented from flowing through the valve assembly to the distal tip.

In some aspects, the techniques described herein relate to a cryobiopsy device, further including: a proximal supply line fluidly connecting the refrigerant cartridge to the valve assembly; and a tube encompassing the proximal supply line between an outer housing of the sampling system and the base housing of the refrigerant system, the tube fluidly connecting an exhaust port of the base housing to an exhaust line located within the outer housing.

In some aspects, the techniques described herein relate to a cryobiopsy device, wherein the sampling system includes a probe assembly extending distally from an outer housing, the probe assembly including: an outer sheath connected to the outer housing; and an inner sheath connected to the position controller, wherein the distal tip is received within, and extends distally beyond, a distal end of the inner sheath.

In some aspects, the techniques described herein relate to a cryobiopsy device, wherein the inner sheath includes a wire positioned to regulate refrigerant flow to the distal tip within the inner sheath.

In some aspects, the techniques described herein relate to a cryobiopsy device, wherein the valve assembly includes a translatable tubular element which opens and closes the valve assembly.

In some aspects, the techniques described herein relate to a cryobiopsy device, wherein an aperture extends through a sidewall of the tubular element, wherein the aperture is aligned with a proximal supply line when the valve assembly is open.

In some aspects, the techniques described herein relate to a cryobiopsy device, wherein the refrigerant controller is translatable in a direction parallel to a longitudinal axis to open and close the valve assembly.

In some aspects, the techniques described herein relate to a cryobiopsy device, wherein the refrigerant controller is engageable with the position controller to lock the valve assembly in an open position.

In some aspects, the techniques described herein relate to a cryobiopsy device, wherein the sampling system includes an outer housing having a position controller that may extend and retract the distal tip.

In some aspects, the techniques described herein relate to a cryobiopsy device, wherein the position controller is configured to guide the distal tip between, and maintain the distal tip in, a retracted position, a partially extended position, and a fully extended position.

In some aspects, the techniques described herein relate to a cryobiopsy device, including: a refrigerant system including a refrigerant cartridge; and a sampling system including: a scope adapter including a connection mechanism; an outer housing translatable between at least a first position relative to the scope adapter in which a distal tip is within an outer sheath and a second position relative to the scope adapter in which the distal tip extends distally beyond the outer sheath; and a refrigerant controller.

In some aspects, the techniques described herein relate to a cryobiopsy device, further including a central body connecting the outer housing to the scope adapter, wherein the outer housing is translatable about the central body to extend and retract the distal tip.

In some aspects, the techniques described herein relate to a cryobiopsy device, further including a pawl assembly, wherein the central body includes a plurality of teeth engageable by the pawl assembly to prevent translation of the outer housing about the central body.

In some aspects, the techniques described herein relate to a cryobiopsy device, wherein the central body is adjustably connected to the scope adapter to adjust a position of a distal end of the cryobiopsy device with respect to a distal end of an endoscope.

In some aspects, the techniques described herein relate to a cryobiopsy device, further including a valve assembly located within the outer housing and in fluid communication with the refrigerant system, the valve assembly movable between an open position in which refrigerant flows through the valve assembly to the distal tip and a closed position in which refrigerant is prevented from flowing through the valve assembly to the distal tip.

In some aspects, the techniques described herein relate to a cryobiopsy device, wherein the valve assembly includes a tubular element translatable in a direction orthogonal to a longitudinal axis of the sampling system to open or close the valve assembly.

In some aspects, the techniques described herein relate to a cryobiopsy device, wherein the valve assembly includes a tubular element translatable in a direction parallel to a longitudinal axis of the sampling system to open or close the valve assembly.

In some aspects, the techniques described herein relate to a cryobiopsy device, wherein the valve assembly includes a plunger translatable in a direction orthogonal to a longitudinal axis of the sampling system to open or close the valve assembly.

In some aspects, the techniques described herein relate to a cryobiopsy device, wherein the valve assembly includes a tube that is deformable to open or close the valve assembly.

In some aspects, the techniques described herein relate to a cryobiopsy device, wherein the valve assembly includes a rotatable element rotatable to open or close the valve assembly.

In some aspects, the techniques described herein relate to a method of performing a cryobiopsy using a cryo-adhesion device, the method including: establishing fluid communication between a refrigerant cartridge and a probe assembly; inserting the probe assembly into a working channel of an endoscope positioned within a patient; directing refrigerant from the refrigerant cartridge through the probe assembly to cool a distal tip; and advancing the distal tip into contact with tissue to cause tissue to adhere thereto.

In some aspects, the techniques described herein relate to a method, wherein establishing fluid communication between the refrigerant cartridge and the probe assembly includes inserting the refrigerant cartridge into a base housing connected to the probe assembly.

In some aspects, the techniques described herein relate to a method, wherein directing refrigerant from the refrigerant cartridge through the probe assembly includes generating an audible alert by venting exhaust gases to the atmosphere through an exhaust port of the base housing.

In some aspects, the techniques described herein relate to a method, wherein directing refrigerant from the refrigerant cartridge through the probe assembly includes replacing the refrigerant cartridge with a second refrigerant cartridge.

In some aspects, the techniques described herein relate to a method, wherein directing refrigerant from the refrigerant cartridge through the probe assembly includes grasping a handle of the cryo-adhesion device and translating a refrigerant controller thereof in a direction parallel to a longitudinal axis of the handle.

In some aspects, the techniques described herein relate to a method, wherein translating the refrigerant controller includes locking the refrigerant controller in an open position.

In some aspects, the techniques described herein relate to a method, wherein advancing the distal tip includes translating a position controller disposed on an outer housing in a direction parallel to a longitudinal axis of the outer housing.

In some aspects, the techniques described herein relate to a method, wherein advancing the distal tip into contact with tissue further includes rotating the position controller about the longitudinal axis.

In some aspects, the techniques described herein relate to a method, wherein the method first includes securing the probe assembly to the endoscope using an endoscope adapter.

In some aspects, the techniques described herein relate to a method, wherein securing the probe assembly to the endoscope includes adjusting position of a distal end of the probe assembly with respect to a distal end of the endoscope using the endoscope adapter.

### BRIEF DESCRIPTION OF THE DRAWINGS

The following figures are included to illustrate certain example aspects of the present disclosure and should not be viewed as exclusive or limiting. The subject matter disclosed is capable of considerable modifications, alterations, combinations, and equivalents in form and function, as will occur to one having ordinary skill in the art and having the benefit of this disclosure. The present disclosure references the drawings as follows:

FIG. 1 is a side view of a cryobiopsy device passing through an endoscope, according to one example of the present disclosure.

FIG. 2 is a cross-section of a gas system, according to one example of the present disclosure.

FIG. 3 is an exploded view of the gas system of FIG. 2, according to one example of the present disclosure.

FIG. 4 is a cross-section of a fluid coupling of the gas system of FIGS. 2-3, according to one example of the present disclosure.

FIG. 5 is a side view of a sampling system, according to one example of the present disclosure.

FIG. 6 is an exploded view of the sampling system of FIG. 5, according to one example of the present disclosure.

FIG. 7 is a cross-section of a refrigerant controller and a first axial tube valve assembly in a closed position, according to one example of the present disclosure.

FIG. 8 is a cross-section of the refrigerant controller and the first axial tube valve assembly of FIG. 7 in an open position, according to one example of the present disclosure.

FIG. 9 illustrates a cross-section of a distal end portion of an outer housing with a fluid connector shown in shadow, according to one example of the present disclosure.

FIG. 10 illustrates a cross-section of a probe assembly, according to one example of the present disclosure.

FIG. 11 illustrates a side view of the probe assembly of FIG. 9 with a distal tip shown partially in shadow, according to one example of the present disclosure.

FIG. 12 is a top view of a position controller in a first position, according to one example of the present disclosure.

FIG. 13 is a cross-section of a probe assembly in a first position, according to one example of the present disclosure.

FIG. 14 is a top view of a position controller in a second position, according to one example of the present disclosure.

FIG. 15 is a cross-section of a probe assembly in a second position, according to one example of the present disclosure.

FIG. 16 is a top view of a position controller in a third position, according to one example of the present disclosure.

FIG. 17 is a cross-section of a probe assembly in a third position, according to one example of the present disclosure.

FIG. 18 is a cross-section of a position controller in first position, according to one example of the present disclosure.

FIG. 19 is a cross-section of a position controller in a second position, according to one example of the present disclosure.

FIG. 20 is a perspective view of a biopsy device, according to one example of the present disclosure.

FIG. 21 illustrates a scope adapter of the cryobiopsy device of FIG. 20 detached from an endoscope, according to one example of the present disclosure.

FIG. 22 illustrates the scope adapter of the cryobiopsy system of FIG. 20 attached to an endoscope according to one example of the present disclosure.

FIG. 23 is a cross-section of the sampling system of FIG. 20, according to one example of the present disclosure.

FIG. 24 is a cross-section of a proximal portion of a sampling system, according to one example of the present disclosure.

FIG. 25 is a cross-section of a central body, according to one example of the present disclosure.

FIG. 26 is a cross-section of probe assembly in a first position, according to one example of the present disclosure.

FIG. 27 is a side view of a sampling system, according to one example of the present disclosure.

FIG. 28 is a cross-section of a probe assembly in a second position, according to one example of the present disclosure.

FIG. 29 is a side view of a sampling system, according to one example of the present disclosure.

FIG. 30 is a side view of a distal portion of a sampling system, according to one example of the present disclosure.

FIG. 31 is a side view of a central body and a scope adapter, according to one example of the present disclosure.

FIG. 32 is a cross section of proximal portion of a sampling system, according to one example of the present disclosure.

FIG. 33 is a cross-section of a second axial tube valve assembly in an open position, according to one example of the present disclosure.

FIG. 34 is a cross-section of a second axial tube valve assembly in a closed position, according to one example of the present disclosure.

FIG. 35 is a cross-section of a first vertical tube valve assembly in a closed position, according to one example of the present disclosure.

FIG. 36 is a cross-section of a first vertical tube valve assembly in an open position, according to one example of the present disclosure.

FIG. 37 is a cross-section of a first plunger valve assembly in a closed position, according to one example of the present disclosure.

FIG. 38 is a cross-section of a first plunger valve assembly in an open position, according to one example of the present disclosure.

FIG. 39 is a cross-section of a second plunger valve assembly in a closed position, according to one example of the present disclosure.

FIG. 40 is a cross-section of a second plunger valve assembly in an open position, according to one example of the present disclosure.

FIG. 41 is a cross-section of a third plunger valve assembly in an open position, according to one example of the present disclosure.

FIG. 42 is a cross-section of a first crush valve assembly in a closed position, according to one example of the present disclosure.

FIG. 43 is a cross-section of a first crush valve assembly in an open position, according to one example of the present disclosure.

FIG. 44 is a cross-section of a second crush valve assembly in a closed position, according to one example of the present disclosure.

FIG. 45 is a cross-section of a second crush valve assembly in an open position, according to one example of the present disclosure.

FIG. 46 is a cross-section of a third crush valve assembly in a closed position, according to one example of the present disclosure.

FIG. 47 is a cross-section of a third crush valve assembly in an open position, according to one example of the present disclosure.

FIG. 48 is a cross-section of a fourth crush valve assembly in an open position, according to one example of the present disclosure.

FIG. 49 is a cross-section of a fourth crush valve assembly in a closed position, according to one example of the present disclosure.

FIG. 50 is a cross-section of a rotatable valve assembly in an open position, according to one example of the present disclosure.

FIG. 51 is a cross-section of a rotatable valve assembly in a closed position, according to one example of the present disclosure.

FIG. 52 is a cross-section of a third vertical tube valve assembly in an open position, according to one example of the present disclosure.

FIG. 53 is a cross-section of a third vertical tube valve assembly in a closed position, according to one example of the present disclosure.

FIG. 54 is a cross-section of a filter housing, according to one example of the present disclosure.

FIG. 55 is a cross-section of a distal tip of a probe assembly, according to one example of the present disclosure.

FIG. 56 is a cross-section of a sampling system including a suction connector, according to one example of the present disclosure.

### DETAILED DESCRIPTION

It will be appreciated by persons skilled in the art that the present disclosure is not limited to what has been particularly shown and described herein. A variety of modifications and variations are possible in view of the teachings herein without departing their scope, spirit, or intent.

While different examples may be described in this specification, it is specifically contemplated that any of the features from the different examples can be used and brought together in any combination. In other words, the features of different examples can be mixed and matched with each other. Hence, while every permutation of features from different examples may not be explicitly shown or described, it is the intention of this disclosure to cover any such combinations, especially as may be appreciated by one of skill in the art.

The terminology used in this disclosure should be interpreted in a permissive manner and is not intended to be limiting. In the drawings, like numbers refer to like elements. Unless otherwise noted, all of the accompanying drawings are not to scale. Unless otherwise noted, the term “about” is defined to mean plus-or-minus 5% of a stated value.

The terms distal or distally generally refer to a direction or area towards an end of a device within a patient (e.g., away from a physician/clinician), while the terms proximal or proximally refer to a direction or area toward an end of a device that remains outside of a patient (e.g., toward or closer to a physician/clinician or handle/hub of a device).

Numerical ranges discussed in this specification should be interpreted as both inclusive numerical ranges and as covering/disclosing a plurality of numbers within the ranges. Specifically, a range should be considered to recite numbers that increment by two decimal places (hundredths) for the purposes of support in the claims (e.g., 0.01, 0.02, 0.03, etc.). Any of these incremented numbers from a range should be understood to have significance and importance in the context of the present specification.

As previously noted above, cryobiopsy devices may improve both the quality and the size of collected tissue samples over traditional forceps biopsy devices. However, current cryobiopsy systems still include some limitations which significantly limit accessibility and applicability. For example, the cooling functionality of existing cryobiopsy probes is enabled and controlled via a connection to a computerized refrigerant control console that is expensive to acquire and requires routine cleaning and maintenance as well as significant amount of dedicated storage space. Still further, current cryobiopsy devices utilize probes which are relatively large in diameter, such as measuring within an inclusive range of about one millimeter to about three millimeters. Such a diameter may prevent such probes from accessing various anatomical locations within a patient, such as deeper locations within the lungs, and may also contribute to the risk of post-collection bleeding and/or infection.

The present disclosure can help to address these issues, among others, such as by providing a cryobiopsy device which eliminates the need for a computerized refrigerant control console and the capital expenditure, ongoing maintenance costs, and storage space requirements associated therewith. Additionally, the cryobiopsy device of the present disclosure may be compatible with a wide variety of existing endoscopes, bronchoscopes, laparoscopes, uroscopes, ureteroscopes, or pleuroscopes, among other systems, so that a new or proprietary scope is not required.

Moreover, by eliminating the need for a traditional computerized refrigerant control console, the cryobiopsy device of the present disclose may be more portable and less expensive than existing devices, and thus cryo-adhesion biopsies or endoscopic ablative procedures may be performed in a wider range of clinical locations to thereby increase patient access to such procedures. Finally, the biopsy system of the present disclosure may include a smaller sampling probe than any existing cryobiopsy or cryoablation probe, such as by defining an outer diameter of less than one millimeter, to enable such procedures to be performed in a wider range of anatomical locations within a patient while concurrently reducing the risk of post-collection bleeding.

Several different novel features of the present disclosure are described below. While some of these novel features may be shown combined together on a single embodiment or device, they may not necessarily be inextricably linked together and thus may be used separately or independently on different embodiments or devices.

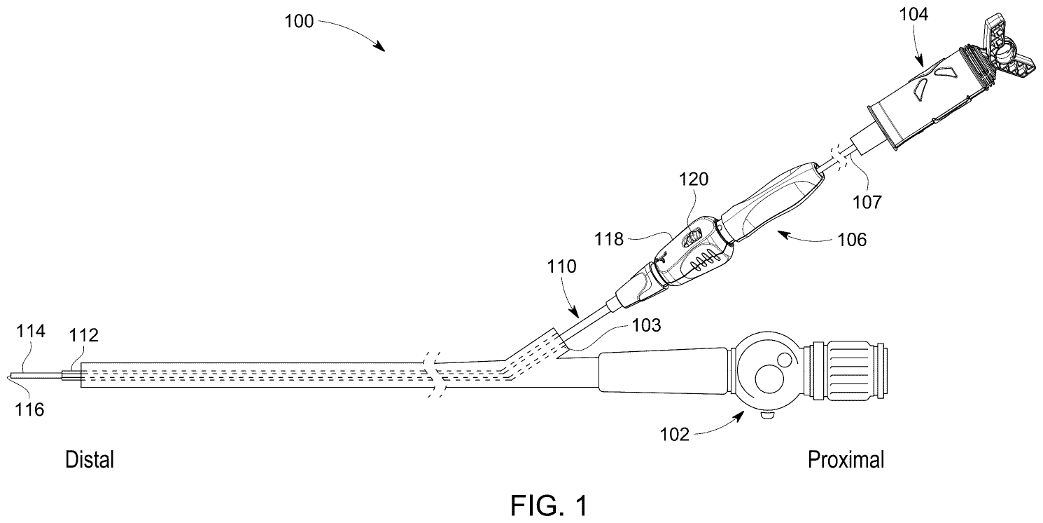

FIGS. 1-20 show various views of a cryobiopsy device 100, according to at least one example of the present disclosure. Turning first to FIG. 1, FIG. 1 shows a side view of a cryobiopsy device 100 passing through a working channel 103 of an endoscope 102. The cryobiopsy device 100 may be a sterile, single-use (e.g., disposable) tissue sampling system that may be used in interventional procedures typically guided by surgical robots through relative long or tortious anatomical insertion paths, such as, but not limited to, to the inner (i.e. central or peripheral) lung nodules.

In some examples, such a surgical robot may be, or may be similar to, the robotic systems and/or devices disclosed in U.S. Pat. No. 7,689,320 or U.S. Pat. Pub. No: 20220313375A1, each of which is hereby incorporated by reference in its entirety. In some examples, such a surgical robot may be the Ion robotic systems from Intuitive Surgical, Inc., of Sunnyvale, California. In another example, such a surgical robot may be the Galaxy robotic system from Noah Medical of San Carlos, California. In a further example, such a surgical robot may be Monarch robotic system from Johnson & Johnson of New Brunswick, New Jersey.

The endoscope 102 may represent a wide variety of different pre-existing flexible or rigid scopes. In some examples, the endoscope 102 may represent a variety of existing scopes defining a working channel defining a diameter within an inclusive range of about 1 millimeter and about 3 millimeters, and a longitudinal length within an inclusive range of about 90 centimeters to about 110 centimeters.

Generally, the cryobiopsy device 100 may include two fluidly connected sub-assemblies: a refrigerant system 104 (FIGS. 1 and 2-4) and a sampling system 106 (FIGS. 1 and 5-20). The refrigerant system 104 houses a single-use refrigerant source, such as a disposable compressed gas cartridge, and is in fluid communication with the sampling system 106 via a tube 107 which may include refrigerant supply and/or exhaust lumens. The sampling system 106 may be a structure, device, or apparatus including an outer housing 108 and a probe assembly 110. The probe assembly 110 may be an elongated structure that may pass through the working channel 103 of the endoscope 102 to collect tissue samples. The probe assembly 110 may be comprised of an outer sheath 112 housing an inner sheath 114 ending in a distal tip 116 which forms a probe for engaging tissue.

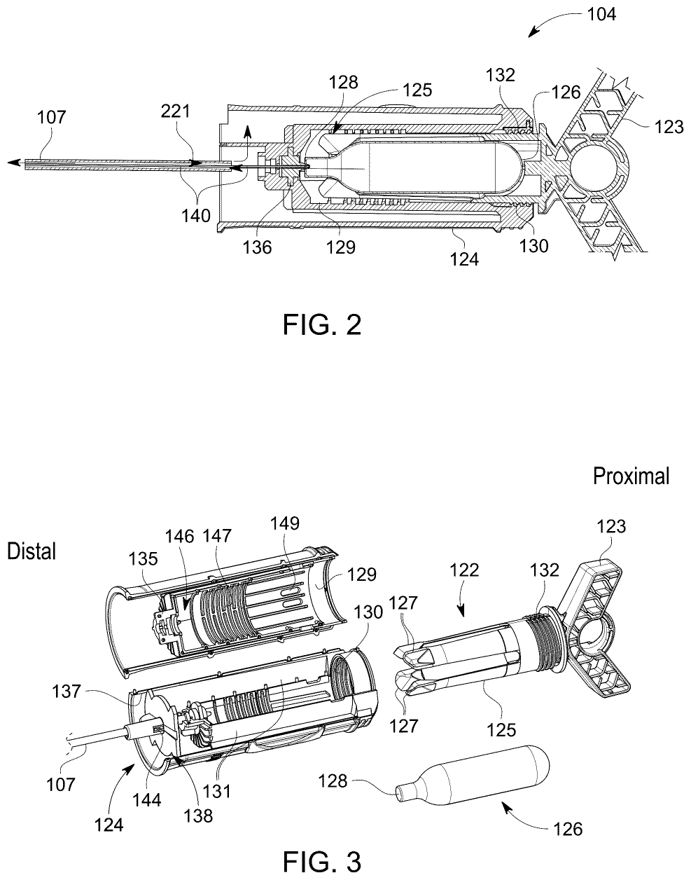

The outer housing 108 may be a generally handle-shaped structure from which the probe assembly 110 distally extends, and which includes a position controller 118 and a refrigerant controller 120 disposed thereon. The position controller 118 advances and retracts the inner sheath 114 and its distal tip 116 relative to the outer sheath 112, and the refrigerant controller 120 starts or stops a flow of refrigerant from the refrigerant system 104 to cool the distal tip 116 and freeze tissue. Thus, the cryobiopsy device 100 does not require a conventional computerized refrigerant supply and/or control console to perform an endoscopic biopsy and/or ablation procedure, for example, to retrieve tissue samples for diagnostic purposes, remove foreign bodies, mucus plugs, blood clots, necrotic tissue, tissue tumors (e.g., palliative recanalization), or otherwise destroy or inactivate diseased tissue. In view of the above, the cryobiopsy device 100 may be more portable, less expensive, and more accessible to both clinicians and patients than existing devices. The cryobiopsy device 100 is described in greater detail below with reference to FIGS. 2-20 and 54.

FIGS. 2-4 show several views of the refrigerant system 104 illustrated in, and described above with reference to, FIG. 1. FIGS. 2-4 are discussed below concurrently. The refrigerant system 104 is a component assembly that may mechanically supply a refrigerant (e.g., a compressed gas) to the sampling system 106 (FIG. 1) in order to, for example, selectively cool the distal tip 116 (FIG. 1) to a temperature suitable for tissue collection via cryo-adhesion or dispense the refrigerant directly into tissue for cryo-ablation. While the refrigerant system 104 is generally described below as being a separate assembly spaced away from the sampling system 106 by the tube 107, it is to be appreciated that the refrigerant system 104, including any of its various components, may alternatively be integrated directly into the sampling system 106. In one such example, the refrigerant system 104 may be contained entirely within the outer housing 108 of the sampling system 106.

The refrigerant system 104 may include a cartridge nest 122 and a base housing 124. The cartridge nest 122 may include a receiving portion 125 sized and shaped to hold a refrigerant cartridge 126 therein. In one such example, the receiving portion 125 may include a plurality of flexible members 127 that may close around the refrigerant cartridge 126 to hold it securely in place therebetween, such as via a snap fit. Alternatively, the receiving portion 125 may instead define a cavity or space to hold the refrigerant cartridge 126, and, in such examples, an end cap, strap, or other retaining feature to retain the refrigerant cartridge 126. In some examples, the cartridge nest 122 may further comprise a handle portion 123. The handle portion 123 may be a proximal-most portion or region sized and shaped to help enable a user to grasp and/or hold the cartridge nest 122, such as when inserting the cartridge nest 122 into, or removing the cartridge nest from, the cartridge nest 122 from, the base housing 124.

The refrigerant cartridge 126 may represent a wide range of different commercially available single-use compressed gas canisters typically having a septum 128 that may be pierced or otherwise broken to release the payload therein. In some examples, the refrigerant cartridge 126 may contain a payload within an inclusive range of about 10 milliliters to about 50 milliliters of compressed gas in a liquid state. In some examples, the refrigerant cartridge 126 may contain a payload within an inclusive range of about 5 grams to about 50 grams of compressed gas. In one specific example, the refrigerant cartridge 126 may contain a payload of 14 grams of compressed gas, which may be sufficient to continuously release gas to the sampling system 106 within an inclusive range of about three minutes to about five minutes of freeze or cooling activation time.

In some examples, the payload of the refrigerant cartridge 126 may generally provide a sufficient amount of refrigerant to complete an entire biopsy procedure. However, in other procedures, the inventors have recognized that a user may need to replace the refrigerant cartridge 126 with one or more replacement gas cartridges to provide additional refrigerant, such as, but not limited to, to obtain relatively large sample sizes or ablate a relatively large amount of tissue.

The refrigerant cartridge 126 may also contain various types of compressed gas, such as, among others, nitrous oxide or carbon dioxide stored in a liquid or gaseous state. In this regard, the inventors have recognized that the use of nitrous oxide may provide the distal tip 116 (FIG. 1) of the sampling system 106 (FIG. 1) with the ability to reach suitable sampling (e.g., cryo-adhesion) temperatures more quickly and/or provide an enhanced ablative effect, as nitrous oxide expands more rapidly than carbon dioxide due to its higher vapor pressure.

Moreover, because nitrous oxide has a lower boiling point (e.g., about −80 degrees Celsius) than carbon dioxide (e.g., about −56 degrees Celsius), the distal tip 116 will cool to a lower temperature when the refrigerant controller 120 (FIG. 1) is in an open position. Still further, the inventors have also recognized that because nitrous oxide has a lower boiling point than carbon dioxide, the use of nitrous oxide as a refrigerant may enable a sampling system connected to the refrigerant system 104 to ablate tissue, such as when a distal tip thereof is hollow and/or defines a bore or passage configured to enable the refrigerant to flow distally therebeyond in contact with tissue.

Additionally, the inventors have recognized that storing and delivering, to the distal tip 116, compressed refrigerant in a liquid state rather than in a gaseous state may substantially reduce the time required to cool the distal tip 116 to a sub-zero temperature for cryo-adhesion purposes as a phase change from a liquid to a gas within or near the distal tip 116 may to help absorb ambient heat, and may also reduce refrigerant consumption to increase the lifespan of the refrigerant cartridge 126 as a reduced refrigerant flow rate may be used without sacrificing cooling functionality.

The base housing 124 may contain the receiving portion 125 of the cartridge nest 122. For example, the base housing 124 may define a chamber 129 into which the receiving portion 125 of the cartridge nest 122 may be inserted. The base housing 124 may also removably engage the cartridge nest 122 via various mechanical coupling techniques known in the art. For example, the base housing 124 may define a first plurality of threads 130 that may threadedly engage a second plurality of threads 132 defined by the receiving portion 125. In some such examples, the first plurality of threads 130 and the second plurality of threads 132 may each include two starts, three starts, or four starts to help maintain the base housing 124 and the cartridge nest 122 in alignment during a start of thread engagement therebetween.

In other examples, the base housing 124 may engage the receiving portion 125 by utilizing other coupling techniques, such as a snap fit or friction fit, removable fasteners such as screws and/or bolts, or a magnetic interface, among others. In still further examples, the cartridge nest 122 may secured to, and/or pressed into the base housing 124 with a system employing a mechanical advantage, such by using a translatable or a rotatable level disposed on the base housing 124 having a cam-style action or linked to a 3 or 4 bar linkage, to help advance the cartridge into the chamber 129 with form to pierce or break a septum of the refrigerant cartridge 126 as discussed further below.

The base housing 124 fluidly connects the refrigerant cartridge 126 to the sampling system 106 (FIG. 1) when the cartridge nest 122 is secured to the base housing 124 within the chamber 129. For example, the base housing 124 may include a fluid coupling 134. The fluid coupling 134 may be an integral structure of the base housing 124, or a separate component assembly, including both a piercing element 136 and a connecting member 138. The piercing element 136 may be a removable or an integral component of the base housing 124 which is hollow, tapered, and/or fluted to pierce the septum 128 of the refrigerant cartridge 126 to begin receiving the compressed payload therein.

The connecting member 138 may be a removable or integral component of the base housing 124 that may receive, or otherwise be connected to, a proximal terminus of the tube 107, and allow a proximal supply line 140 originating either within the piercing element 136 or within geometry of the housing in fluid communication with the piercing element 136 to pass into the tube 107. Further, in this regard, the connecting member 138 may fixedly or detachably interface with the proximal terminus of the tube 107 through various coupling techniques known in the art, such via an adhesive or reflow bond therebetween, or through a mechanical engagement mechanism such as a barbed tube nipple, a circumferential tube clamp, or one or more screws or and/or bolts, among others.

In some examples, the cartridge nest 122 may be adapted to hold a plurality of gas cartridges. For example, the receiving portion 125 of the cartridge nest 122 may be adapted to include a plurality of spaces or cavities each sized and shaped to receive an individual gas cartridge. In some examples, such a plurality of spaces or cavities may comprise two, three, four, or other numbers of spaces or cavities to hold two, three, four, or other numbers of gas cartridges, respectively. In such examples, the piercing element 136 and the connecting member 138 may also be configured such that a user may insert the cartridge nest 122 into the base housing 124 to simultaneously establish fluid communication between each of the plurality of gas cartridges and the proximal supply line 140. For example, the piercing element 136 may comprise a plurality of hollow, tapered, and/or fluted members positioned to concurrently pierce the septum 128 of each of a plurality of gas cartridges held within the receiving portion 125, and the connecting member 138 may include a splitter, plenum, or other feature adapted to fluidly connect each of the hollow, tapered, and/or fluted members of the piercing element 136 to the proximal supply line 140.

Once within the tube 107, the proximal supply line 140 continues distally therethrough and into the sampling system 106 (FIG. 1). Notably, in FIG. 2, the connecting member 138 is removed to help illustrate the proximal supply line 140 entering the tube 107. The proximal supply line 140 may be a high-pressure line sized and shaped to maintain refrigerant in a liquid state as it flows thereinto, such as to prevent the refrigerant from losing any of its cooling potential. In some examples, the proximal supply line 140 may define an outer diameter within an inclusive range of about 0.008 to about 0.030 inches. In one specific example, the proximal supply line 140 may define an outer diameter of about 0.022 inches.

In some examples, the proximal supply line 140 may define an inner diameter within an inclusive range of about 0.008 to about 0.025 inches. In one specific example, the proximal supply line 140 may define an inner diameter of about 0.016 inches. Of course, the inventors have recognized that the inner and outer diameter of the proximal supply line 140 may vary depending upon, for example, the type of refrigerant used, whether the refrigerant is intended to be maintained in a liquid state or allowed to expand to a gaseous state, or the longitudinal distance to the sampling system 106, among other factors. The proximal supply line 140 may be constructed from various suitably resilient and/or pressure resistant materials such as, but not limited to, braided, woven, or solid stainless steel, titanium, nitinol, or coated or non-coated steel tubing, or alternately braided, woven, or solid polymeric material such as, but not limited to, polyamide, polyether ether ketone, ethylene tetrafluoroethylene, polytetrafluoroethylene, or polyvinylidene fluoride, among others.

As previously mentioned, the proximal supply line 140 passes through the tube 107 to supply the sampling system 106 with a flow of refrigerant from the refrigerant cartridge 126, such as indicated by arrow 220 in FIG. 2. In this regard, the tube 107 also functions as a return lumen for returning exhaust gases, such as shown by arrow 221 in FIG. 2, from the sampling system 106 to the refrigerant system 104. As the tube 107 carries expanded gases, the tube 107 may, in some examples, be constructed of a less pressure resistant and more flexible material relative to the proximal supply line 140, such as, but not limited to, polyurethane, polyamide, or rubber, among others. Moreover, in some examples, the material used for the tube 107 and/or the proximal supply line 140 may be configured to ensure that the tube 107 and the proximal supply line 140 can flex to some extent to allow the refrigerant system 104 and the sampling system 106 (FIG. 1) to be manipulated relative to each other. In some such examples, the tube 107 may be at least partially made from a braided material or may otherwise include one or more reinforcing elements, such as coils or stringers, to help improve kink resistance.

In some examples, the tube 107 and the proximal supply line 140 therein may define an overall length with an inclusive range of between about 2 feet and about 8 feet. In one specific example, the tube 107 may define a length measuring about 5 feet. In some examples, the tube 107 may define an inner diameter within an inclusive range of about 0.07 inches to about 0.12 inches. In one example, the tube 107 may define an inner diameter of about 0.09327 inches. In some examples, the tube 107 may define an outer diameter within an inclusive range of about 0.12 inches to about 0.17 inches. In one example, the tube 107 may define an outer diameter of about 0.15625 inches. Generally, the proximal supply line 140 may define a slightly greater overall length than the tube 107, such as, for example, but not limited to, within an inclusive range of about 2 inches to 6 inches greater, at least by virtue of extending farther into the sampling system 106 and/or the base housing 124.

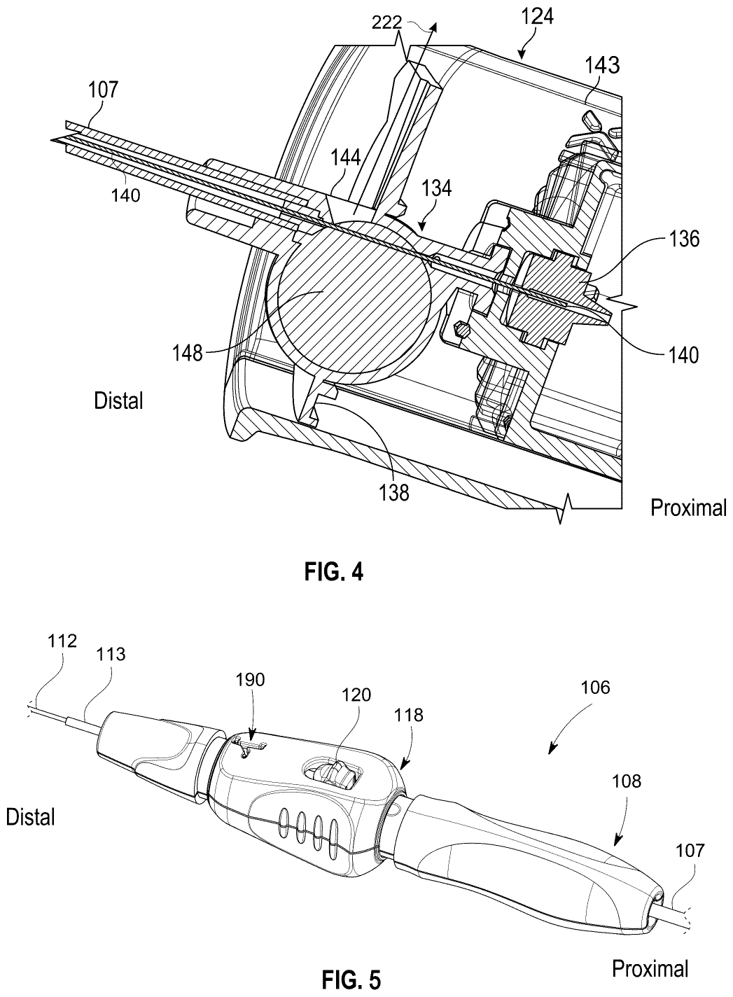

Turning back to the base housing 124, the connecting member 138 thereof may also be sized and shaped to allow exhaust gases returning from the sampling system 106 through the tube 107 to vent into the atmosphere. For example, the connecting member 138 may further define an exhaust port 144. The exhaust port 144 may generally be a passage sized and shaped to vent exhaust gases in a direction away from the hands of the user, such as distally away from the handle portion 123 of the cartridge nest 122 and/or into a non-permeable sidewall of the base housing 124 to ensure the user is not directly exposed to the exhaust gases. In one example, the exhaust port 144 may be sized and shaped to direct exhaust gases in a distal direction at an angle within an inclusive range of about 5 degrees and about 90 degrees relative to a central axis A1 of the base housing 124. In some examples, the exhaust port 144 may be sized and shaped to direct exhaust gases into a distal chamber 143, such as shown by arrow 222 in FIG. 4, of the base housing 124 located distally to the chamber 129 of the base housing 124, such as to help contain and/or disperse the exhaust gases as they leave the exhaust port 144.

In some examples, the exhaust port 144 may also include, or may be fluidly connected to, a resistance control mechanism adapted to allow a user to selectively obstruct the exhaust port 144 to increase exhaust backpressure to, in turn, help limit or control the size of an ice ball created in tissue at or near the distal tip 116, as the inventors have recognized that increasing the exhaust backpressure may increase the boiling point of the refrigerant and thus reduce a maximum size of the ice ball created in tissue. In various examples, the exhaust backpressure may be selected or otherwise configured based on the physical characteristics of the tissue to be sampled, the desired tissue sample size, and/or environmental factors such as ambient heat or natural ground elevation (e.g., height above sea level) that may affect the natural flow rate of exhaust gases.

In some such examples, the tube 107 may be made from a deformable material and the connecting member 138 may include or contain a pivotable, slidable, rotatable, or otherwise movable element configured to enable a user to impart a crush force to the tube 107 at or near its proximal terminus, such as to enable a user to selectively reduce the internal diameter and increase exhaust backpressure within the tube 107. In other examples, the connecting member 138 may include or contain a rotatable mechanism adapted to enable a user to rotate a plurality of differently sized apertures or nozzles into the exhaust flow path within the tube 107 to selectively restrict or limit the flow rate of exhaust gases passing through the exhaust port 144.

In some examples, the base housing 124 may further include a vent pathway 146 configured to improve user safety during insertion and/or withdrawal of the cartridge nest 122. For example, when the septum 128 is broken by the piercing element 136 during insertion of the cartridge nest 122 into the base housing 124, or when the refrigerant cartridge 126 disengages the piercing element 136 during withdrawal of the cartridge nest 122 from the base housing 124, a relatively limited or small volume of gas may be released into the chamber 129 and immediately vent proximally and toward the user's hand on the handle portion 123. In order to prevent this occurrence, the base housing 124 may define a vent pathway 146 within the chamber 129 which includes a series of baffles 147 that may direct escaping gas through a highly tortuous pathway to at least one outlet 149 defined through a sidewall of the base housing 124 to thereby slow down and/or deflect the gas away from the handle portion 123 of the cartridge nest 122.

In some examples, such as in examples where the base housing 124 may be a two-piece assembly comprising of a first portion 135 and a second portion 137, one of the top or bottom portions may include a pair of flanges 131 sized and shaped to ensure that the first portion 135 and the second portion 137 mate asymmetrically to help prevent gas from escaping the base housing 124 through a gap between the first portion 135 and a second portion 137 and bypassing the vent pathway 146.

In some examples, the connecting member 138 may further define a whistle chamber 148 located between the tube 107 and the exhaust port 144. The whistle chamber may be configured (e.g., sized and shaped) to cause flowing exhaust gases (e.g., when the refrigerant controller 120 (FIG. 1) is in an “on” position) to generate an audible whistling sound or alert tone through aerodynamic oscillation therewithin. In some examples, the whistle chamber 148 may also contain a ball or other freely movable element to thereby produce a trilling sound similar to a commercially available pea-whistle.

Moreover, the inventors have recognized that the sound or tone emitted from the whistle chamber 148 will change in proportion to the flow rate of exhaust gas exiting through the exhaust port 144, and correspondingly, the rate of liquid refrigerant flowing out of the refrigerant cartridge 126. Thus, in view of the above, the whistle chamber 148 may produce an audible signal capable of allowing a user to audibly monitor, and/or act to consciously conserve such as by adjusting a position of the refrigerant controller 120 (FIG. 1), the amount of refrigerant being consumed while performing a surgical procedure.

However, in other examples, the refrigerant system 104 may further include a continuous flow regulator, such as positioned proximally to the connecting member 138 and/or within the tube 107, adapted to down regulate the flow rate of exhaust gases into the whistle chamber 148. In such examples, the audible signal emitted by the whistle chamber 148 may be a constant or otherwise unchanging tone irrespective of the position of the refrigerant controller 120 (e.g., if the refrigerant controller 120 is in a fully open position or in a partially open position) or the ambient environmental conditions such as temperature or natural ground elevation (e.g., height above sea level) that may otherwise affect the natural flow rate of exhaust gases through the tube 107 and/or the connecting member 138.

In some examples, the base housing 124 may further include an insulating jacket or layer surrounding or encompassing at least a portion of an outer circumference or outermost perimeter of the base housing 124 to provide additional thermal protection for a user's hands. In some examples, such an insulating jacket or layer may be formed from various insulative materials, such as including, but not limited to, a closed cell foams such as polyethylene foam, polyurethane foam, ethylene-vinyl acetate foam, polychloroprene foam, or polyvinyl chloride foam, among others.

In some examples, the base housing 124 may also incorporate a visible gauge, connected to a scale adapted to weigh the refrigerant cartridge 126, in fluid communication with the piercing element 136, the connecting member 138, and/or the proximal supply line 140 in order to visually display to a user an estimated amount of liquid and/or gaseous payload remaining in the refrigerant cartridge 126. In some examples, the base housing 124 may also include an electric heater and/or temperature regulation mechanism configured to counteract the natural cooling of the refrigerant cartridge 126 as its payload flows distally.

As those skilled in the art will appreciate, preventing the refrigerant cartridge 126 from cooling and/or increasing the temperature of the refrigerant cartridge 126 may improve refrigerant flow through the proximal supply line 140 carrying the refrigerant to the sampling system 106. In this regard, in some examples, the cartridge nest 122 may further be configured to help retain ambient heat to further warm the refrigerant cartridge 126. For example, the cartridge nest 122 may include one or more metallic portions and/or heatsinks positioned to contact the refrigerant cartridge 126 when the refrigerant cartridge 126 is received within the cartridge nest 122.

Additionally, with respect to improving flow characteristics within the proximal supply line 140, the base housing 124 may also be positioned in a vertical orientation to optimize liquid flow from the refrigerant cartridge 126 and through the proximal supply line 140. For example, the base housing 124 may be hung or otherwise suspended from a pre-existing intravenous bag (“IV”) support pole, a proprietary or pre-existing structure, a surgical robot, and/or clinical personnel so that the septum 128 faces downwardly toward the ground or the surface on which the support pole or structure is positioned. Additionally, to help facilitate such positioning, the base housing 124 may further include one or more hooks, clips, suction cups, or other features that may help a user attached the base housing 124 in a vertical orientation to a supporting structure, surgical robot, or clinical personnel.

Finally, with respect to the refrigerant system 104, the cartridge nest 122 and the base housing 124, including various components thereof such as, but not limited to, the first portion 135, the second portion 137, and/or the connecting member 138, may be comprised of various materials, such as including, but not limited to, polymeric materials including acrylonitrile butadiene styrene, polyamide, or high impact polystyrene, among others. In some examples, other components of the refrigerant system 104, such as including, but not limited to, the piercing element 136, may be comprised of metallic materials including, but not limited to, stainless steel, titanium, brass, or coated or non-coated steel, or alternatively non-metallic metallic materials including, but not limited to, plastics such as polyether ether ketone (“PEEK”), glass filled poly ether ketone, carbon filled poly ether ketone, or polyetherimide (“Ultem”).

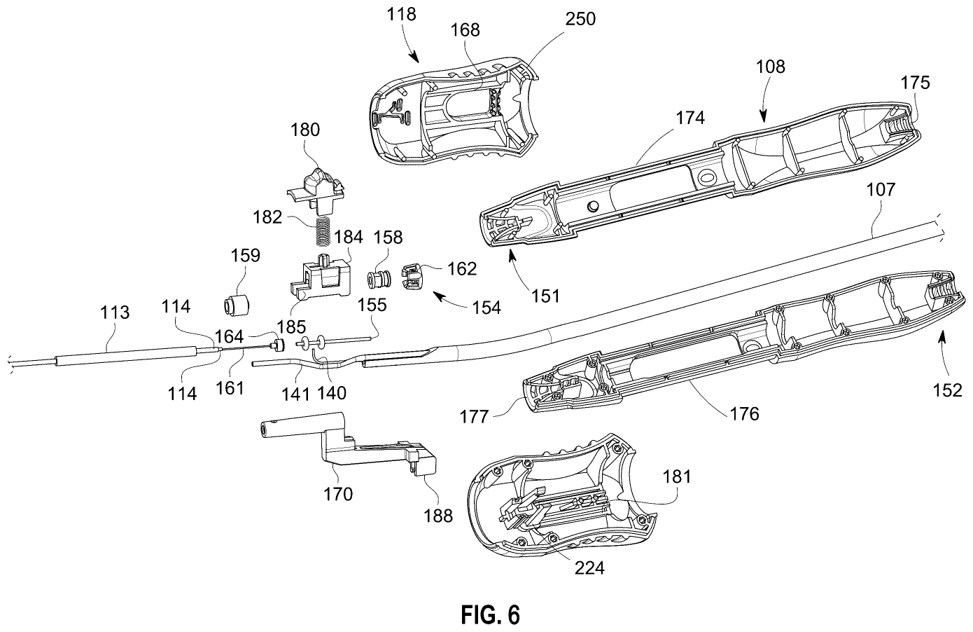

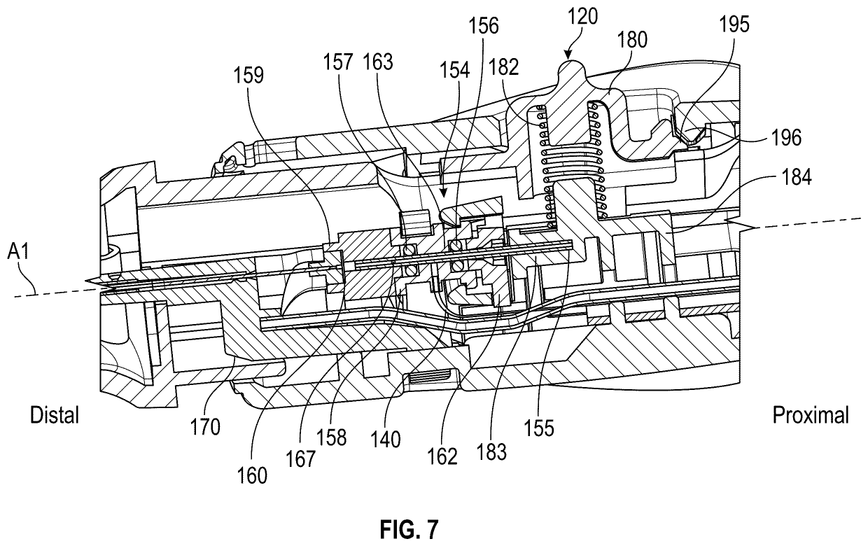

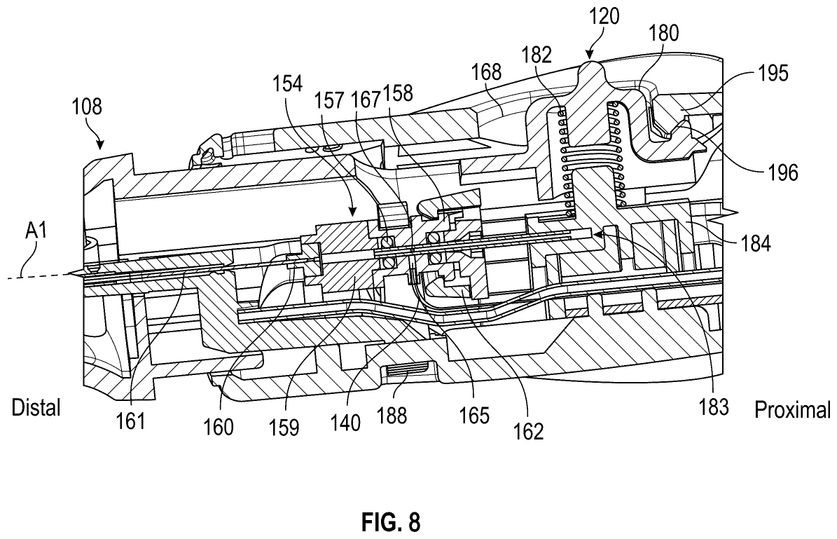

FIGS. 5-19 and 54 show various views of the sampling system 106 illustrated in, and described above with reference to, FIG. 1. FIGS. 7-8 further show a longitudinal axis A1 of the sampling system 106. FIGS. 5-19 are discussed below concurrently. As shown in FIG. 5, the sampling system 106 includes the outer housing 108 and the probe assembly 110. The outer housing 108 forms a generally handle-shaped structure with a proximal end portion 151 (FIG. 6) and a distal end portion 152 (FIG. 6). In some examples, such as shown in FIG. 6, the outer housing 108 may be a two-piece assembly comprising of a first portion 174 and a second portion 176.

As shown in FIG. 6, the tube 107 (FIGS. 5-6), with the proximal supply line 140 therein, may pass through an aperture in the proximal end portion 151 of the outer housing 108 and into the sampling system 106. In some examples, such an aperture may be a flared proximal passage 175 (FIG. 6) having a trumpet-like shape configured (e.g., sized and shaped) shaped to help reduce stress on the proximal supply line 140 and/or the tube 107, such as during manipulation of the refrigerant system 104 (FIGS. 1-4) and the outer housing 108 relative to each other, by progressively engaging the tube 107 to limit axial deflection and/or bending of the tube 107 to a gradual curvature. Such a configuration may help to prevent kinking or breakage of the tube 107 and the proximal supply line 140 passing there though.

Generally, a distal terminus of the tube 107 may be positioned within the outer housing 108 in a location proximal to a distal terminus of the proximal supply line 140. In this respect, it is appreciated that the tube 107 may be potted with an adhesive or sealing material at the location where the proximal supply line 140 exits the tube 107 to prevent leakage of the exhaust gases which, as previously noted, travel proximally through the tube 107 to the refrigerant system 104 for venting to the atmosphere. However, in other examples, the proximal end portion 151 of the outer housing 108 may detachably interface with the proximal supply line 140 and the tube 107, such through barbed tube nipples, circumferential tube clamps, or a plurality of screws or and/or bolts, among others.

As also previously noted, the sampling system 106 includes the refrigerant controller 120, which starts or stops the flow of refrigerant from the refrigerant system 104 into the sampling system 106 via the proximal supply line 140, and the position controller 118, which extends and retracts the inner sheath 114 and the distal tip 116 relative to the outer sheath 112 via an interface with the inner sheath 114. In the examples of FIGS. 5-19, the position controller 118 may be a translatable structure, or component assembly, disposed at least partially around the outer housing 108, and the refrigerant controller 120 may be a translatable structure, or component assembly, disposed at least partially within the position controller 118 and/or the outer housing 108. Detailed aspects of the refrigerant controller 120 are described first below.

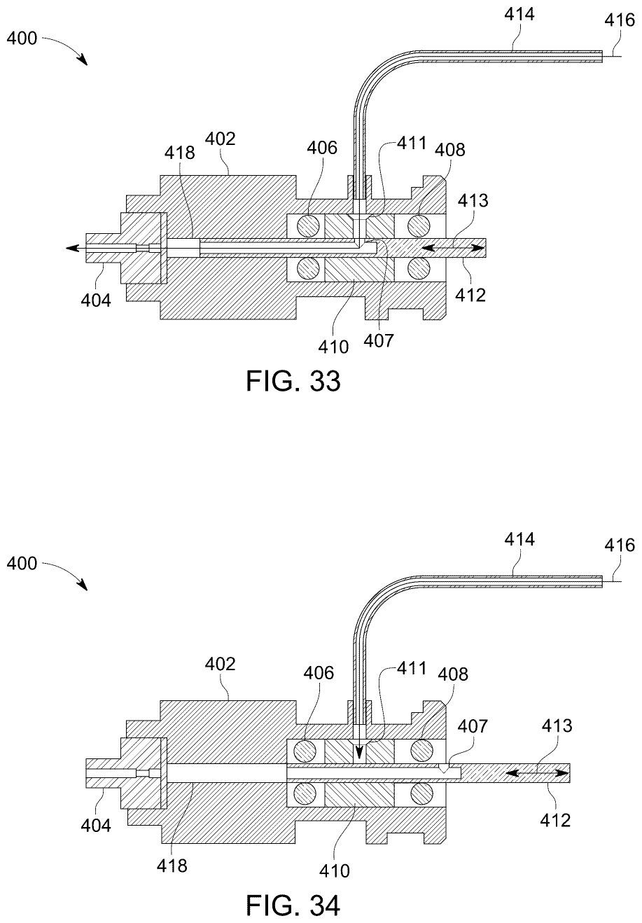

The refrigerant controller 120 may be enabled to control the flow of refrigerant from the refrigerant system 104 by opening and closing a valve assembly fluid connecting the proximal supply line 140 to a distal supply line 161 which carries the refrigerant distally onward to the distal tip 116. In some examples, the valve assembly may be the first axial tube valve assembly 154 shown in at least FIGS. 6-8. In other examples, alternative valve assemblies may be used, such as including, but not limited to, the valve assemblies illustrated in, and described with reference to, FIGS. 33-53 below. The first axial tube valve assembly 154 may be comprised of, as best shown in FIGS. 6-9, a tubular element 155, a first seal 156, a second seal 157, a valve body comprising a first body portion 158 and a second body portion 159, a filter 160, an end cap 162, and a filter cap 164.

The first body portion 158 may be fluidly connected to a distal terminus of the proximal supply line 140 and secured to the position controller 118 via a mounting boss 224 (FIG. 6). In this regard, the mounting boss 224 may, in some examples, be configured to break away from the first body portion 158 and/or the position controller 118 upon receiving or experiencing a pre-determined force. For example, if a user pulls on the outer housing 108 during a surgical procedure, the mounting boss 224 may break at a predetermined force that is less than a predetermined force at which the distal tip 116 may detach from the inner sheath 114 to thereby foreclose the possibility of refrigerant leaking into a location within a patient in the event that excessive withdrawal forces are applied to the sampling system 106.

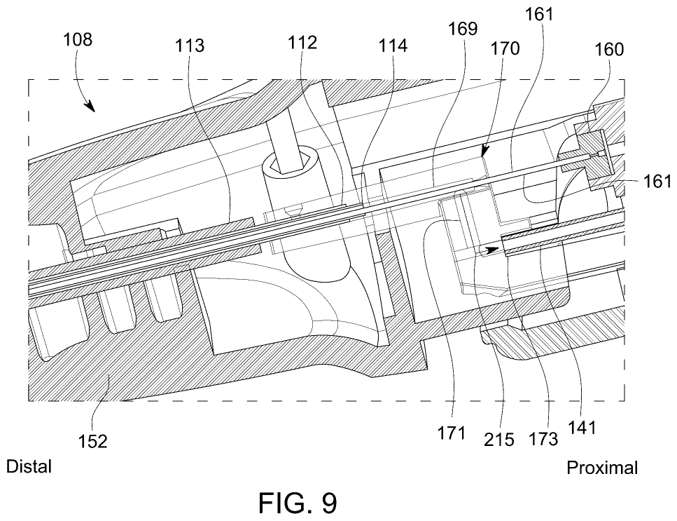

The first body portion 158 may also define a first passage 163 (FIG. 8) for guiding translation of the tubular element 155 in a direction parallel to the longitudinal axis A1, and a second passage 165 (FIG. 9) in fluid communication with the proximal supply line 140. The first seal 156 and the second seal 157 are positioned on opposite sides of the second passage 165 within the first body portion 158 and are sized and shaped to seal around an outer circumference of the tubular element 155. Further, the first seal 156 and the second seal 157 are comprised of a compliant gasket material capable of creating an air-tight seal, such as, but not limited to, polymeric materials such as, but not limited to, rubber or other elastomeric materials including, but not limited to, ethylene propylene diene monomer (“EPDM”), silicone, or nitrile, among others.

Thus, compressed gas may escape the first body portion 158 only by passing through the tubular element 155. In this regard, the tubular element 155 may define an aperture 167 (FIGS. 7-8) extending through its sidewall to enable gas from the proximal supply line 140 to enter the tubular element 155 when the aperture 167 is aligned with the second passage 165 and thereby the proximal supply line 140. Additionally, a proximal end of the tubular element 155 may be sealed to prevent refrigerant from escaping through a proximal terminus of the tubular element 155.

As such, when the aperture 167 of the tubular element 155 is axially aligned with the proximal supply line 140, as shown in FIG. 8, refrigerant such as in liquid form, is free to flow through the tubular element 155 and distally therebeyond. Thus, in such a state, the first axial tube valve assembly 154 is in an “open” position. Conversely, when the aperture 167 is located proximally to the first seal 156, such as shown in FIG. 7, refrigerant is retained between the first seal 156 and the second seal 157. Thus, in such a state, the first axial tube valve assembly 154 is in a “closed” position.

Finally, in some examples, the aperture 167 may form an elongated shape, such as in the form of a rectangular slit or an ellipsoidal opening, to provide for variable flow control. In other words, a user may completely or entirely align the aperture 167 with the second passage 165 to thereby select a maximum flow rate through the tubular element 155, or a user may partially align the aperture 167 with the second passage 165 to thereby select a reduced the flow rate. Such a configuration may be, for example, beneficial for selecting the speed at which the distal tip 116 cools and/or a desire to conserve refrigerant, such as depending upon the physical characteristics of the tissue, the desired tissue sample size, and/or environmental factors such as ambient heat or natural ground elevation (e.g., height above sea level) that may affect the natural flow rate of liquid or gaseous refrigerant.

The end cap 162 may be a structure sized and shaped to proximally abut and engage the first body portion 158 to thereby retain the first seal 156 within the first body portion 158, and the second body portion 159 may be a structure sized and shaped to distally abut the first body portion 158 to thereby retain the second seal 157 within the first body portion 158. Additionally, as shown in at least FIGS. 7-8, the end cap 162 and the second body portion 159 also define respective passages axially aligned with the first passage 163 to provide sufficient room for the tubular element 155 to move between its proximal, open position and its distal, closed position unobstructed. The second body portion 159 may receive the filter 160 and the filter cap 164. The filter 160 itself may be a structure or component assembly configured to prevent undesirable particulates and/or other contaminates which may be inadvertently introduced into, or may naturally accumulate within, the proximal supply line 140, from flowing into and potentially clogging liquid flow through the probe assembly 110. The filter cap 164 may be bonded, laser welded, adhered, or otherwise secured within the second body portion 159 to secure the filter 160 therewithin.

The filter 160 may contain, for example, but not limited to, a mesh or woven, polymeric or metallic filter element, or alternately may comprise a sintered metallic filter element. In some examples, the filter 160 may be completely, or partially, made from stainless steel, coated steel, titanium, polyurethane, polyethylene terephthalate (“PET”), polytetrafluoroethylene, or expanded polytetrafluoroethylene (“ePTFE”), or polypropylene, among other materials. In some examples, a filter element within the filter 160 may be configured to have pore sizes measuring within an inclusive range of about 1 micron to about 50 microns. Positioning the filter 160 downstream of the first axial tube valve assembly 154 and the proximal supply line 140 may be beneficial as such a configuration enable the flow of refrigerant to be filtered at a distal-most, or otherwise final, location before said gas flow enters the probe assembly 110.

As the refrigerant controller 120 starts and stops the flow of refrigerant through any valve assembly that may be contained within the sampling system 106, it logically follows that, in examples of FIGS. 5-19, the refrigerant controller 120 may be connected to the tubular element 155 so that movement of the refrigerant controller 120 results in corresponding axial translation of the tubular element 155. In some examples, the refrigerant controller 120 may be a simple slidable structure directly connected to the tubular element 155.

In other examples, such as shown in FIGS. 7-8, the refrigerant controller 120 may be a component assembly comprised of an activation button 180, a spring 182, and a sliding block 184. The activation button 180 may be a user-engageable component that may be exposed through an aperture 168 defined in the position controller 118. The spring 182 may extend between, and functionally connect, the sliding block 184 and the activation button 180. For example, the activation button 180 may define a first spring projection 187 (FIG. 8) and the sliding block 184 may define a second projection 189 (FIG. 8) each sized and shaped to extend into an inner diameter of the spring 182.

The spring 182 may also bias the activation button 180 upwardly and away from the sliding block 184 so that the activation button 180 naturally stays in continuous contact with surface geometry of the position controller 118 and/or outer housing 108. The sliding block 184 may be a structure that may interface with the tubular element 155 and slide proximally and distally within the outer housing 108 in a direction parallel to the longitudinal axis A1. For example, the sliding block 184 may include a recess 183 (FIGS. 7-8) within with a proximal end portion of the tubular element 155 may be bonded and/or sealed, and a base 185 sized and shaped to be retained and guided by a track 181 (FIG. 6) defined in the position controller 118. Thus, in view of the above, a user may translate the activation button 180 proximally to cause the sliding block 184 to open the first axial tube valve assembly 154 or translate the activation button 180 to cause the sliding block 184 to close the first axial tube valve assembly 154.

In some examples, the refrigerant controller 120 may further prevent inadvertent opening of the first axial tube valve assembly 154, among other valve assemblies, by way of including additional geometric features sized and shaped to engage corresponding geometric features on the position controller 118. For example, the activation button 180 may define a first angled surface 195 (FIG. 7) shaped to contact and engage a second angled surface 196 (FIG. 7) of the position controller 118; and the activation button 180 may further define a third angled surface 197 (FIG. 8) shaped to contact and engage a fourth angled surface 198 (FIG. 8) of the position controller 118.

In such an example, the first angled surface 195 may be normally biased, such as shown in FIG. 7, against the second angled surface 196 by the spring 182 when the refrigerant controller 120 is in a closed position to thereby prevent the activation button 180 from being translated proximally. However, if the activation button 180 is first depressed (i.e., moved in direction orthogonal to the longitudinal axis A1) toward the sliding block 184, the first angled surface 195 will disengage the second angled surface 196, thereby allowing the activation button 180 to be translated proximally.

Subsequently, if the activation button 180 is then released in a position where the third angled surface 197 is proximal to the second angled surface 196, and/or axially aligned with the fourth angled surface 198 on the position controller 118, the spring 182 will cause the activation button 180 to return upwardly and thereby drive the third angled surface 197 into engagement with the fourth angled surface 198, such as shown in FIG. 8, and thereby locking the refrigerant controller 120 in an open position without the application of continuous pressure by a user. Additionally, due to the nature of the engagement between the third angled surface 197 and the fourth angled surface 198, when the user wishes to return the refrigerant controller 120 to a closed position, the activation button 180 may simply be pushed or translated distally, without first being depressed, to cause the third angled surface 197 to disengage the fourth angled surface 198 and the first angled surface 195 to subsequently re-engage the second angled surface 196.

In still further examples, the sampling system 106 may be configured to self-vent excess refrigerant within the first axial tube valve assembly 154, or the valve assemblies illustrated in, and described with reference to, FIGS. 33-53 below. For example, a flip valve, a relief valve, or various other types of pressure-venting or bleeding mechanisms may be located within the outer housing 108 or the position controller 118 and configured such that when the refrigerant controller 120 is moved to a closed position, the distal supply line 161 may immediately vent pressure contained therein, and thus divert excess refrigerant, into the outer housing 108 or the position controller 118 rather than allowing any remaining refrigerant contained therein to flow distally to the distal tip 116.

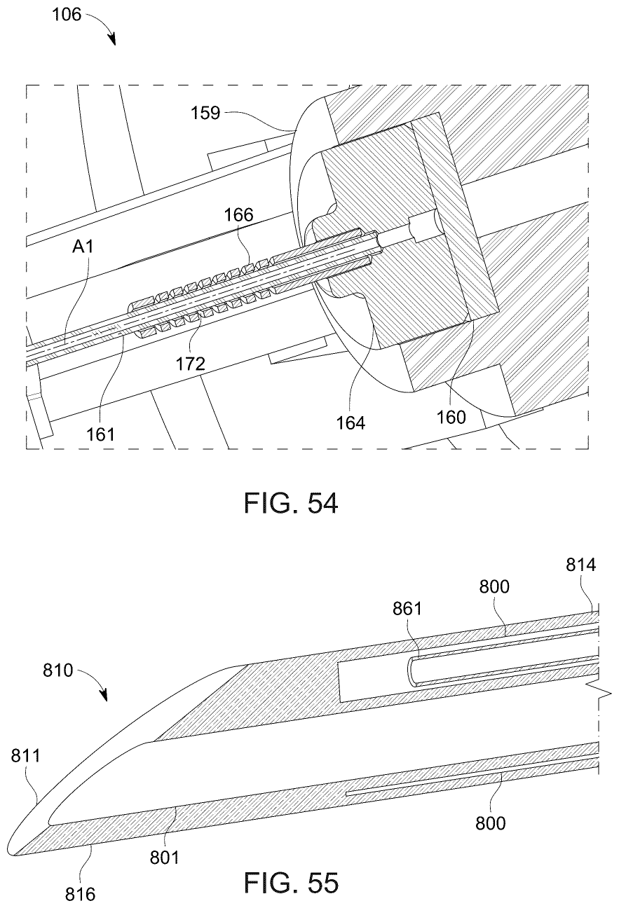

Turning now to the probe assembly 110, after the refrigerant passes through the filter 160 and/or the filter cap 164 from a valve assembly, it enters a distal supply line 161 extending distally from, and bonded, adhered, laser-welded, or otherwise sealed to, the filter cap 164. In some examples, such as shown in FIG. 54, the proximal terminus of the distal supply line 161 may be secured to the filter cap 164 via a sleeve element 166 (FIG. 54). The sleeve element 166 may be adapted to help facilitate a secure and longitudinally lengthened bond between the filter cap 164 and the distal supply line 161 while increasing the axial strength and maintaining deflective flexibility of the distal supply line 161.

For example, the sleeve element 166 may be bonded, laser-welded, adhered, reflowed, or otherwise secured to an outer surface or circumference of the distal supply line 161, and an inner surface or circumference of the filter cap 164 may be bonded, laser-welded, adhered, reflowed, or otherwise secured to an outer surface or circumference of the sleeve element 166. Further, the sleeve element 166 may include a plurality of slots 172 adapted to increase the flexibility (e.g., decrease the resistance to axial deflection) of the sleeve element 166 (FIG. 54). Moreover, the plurality of slots 172 may be conducive or may help to wick solder or adhesive axially along the distal supply line 161 to further increase the strength of the bond therebetween. In some examples, the sleeve element 166 may be made from metallic materials including, but not limited to, stainless, titanium, or nitinol.

The distal supply line 161 then extends to its distal terminus located within, or adjacent to, the distal tip 116. The distal supply line 161 is, like the proximal supply line 140, configured to prevent the compressed liquid refrigerant from expanding and/or transitioning into a gaseous state therewithin. As such, the distal supply line 161 may define an inner diameter that is similar to, or smaller than, the inner diameter of the proximal supply line 140, such as within an inclusive range of about 0.004 to about 0.010 inches. In one specific example, the distal supply line 161 may define an inner diameter of about 0.006 inches in diameter.

Moreover, in this regard, the inventors have appreciated that the inner diameter of all tubes and/or passages containing a liquid or gaseous refrigerant within the cryobiopsy device 100 should either be equal, or should step down or otherwise shrink in diameter, from a proximal direction to a distal direction within the cryobiopsy device 100 to help prevent compressed refrigerant from expanding and/or transitioning into a gaseous state. Accordingly, in some examples, an inner diameter of the distal supply line 161 may be less or equal to an inner diameter of the tubular element 155, and the inner diameter of the tubular element 155 may be less than or equal to an inner diameter of the proximal supply line 140. In some examples, the tubular element 155 may define an inner diameter within an inclusive range of about 0.010 inches to about 0.02 inches. In some specific examples, the tubular element 155 may define an inner diameter measuring about 0.015 inches or 0.016 inches.

The distal supply line 161 may, in some examples, define an outer diameter within an inclusive range of about 0.006 inches to about 0.014 inches. In one specific example, the proximal supply line 140 may define an outer diameter of about 0.012 inches. Still further, as the distal supply line 161 is, like the proximal supply line 140, that may maintain gas at relatively high pressures, it may also be constructed from a similarly pressure resistant material, such as, but not limited to, braided, woven, or solid stainless steel, titanium, nitinol, or coated or non-coated steel tubing, or braided, woven, or solid polymeric material such as, but not limited to, polyamide, polyimide, polyether ether ketone, or ethylene tetrafluoroethylene, or polytetrafluoroethylene, or polyvinylidene fluoride, among others.

The distal supply line 161 may define various longitudinal lengths, such as within an inclusive range of about 115 centimeters and about 130 centimeters. In one example, the distal supply line 161 may define a longitudinal length of about 125 centimeters. In this regard, the longitudinal length and/or inner and outer diameters of the probe assembly 110, including the distal supply line 161, the outer sheath 112, the inner sheath 114, among others components, may vary depending upon the size and length of the working channel 103 (FIG. 1) of the endoscope 102 (FIG. 1) and/or the endoscopic procedure (e.g., the anatomical location to be accessed and/or physical characteristics of the tissue to be sampled). In further examples, the inner diameter of the outer sheath 112 and outer diameter of the inner sheath 114 may be selectively configured to provide a significant air gap therebetween, such as sized to enable the distal tip 116 to be retracted into the outer sheath 112 with a tissue sample attached en face to, or circumferentially around, the distal tip 116. In such an example, the tissue sample may be protected from the possibility of being crushed during withdrawal of the sampling system 106 through an endoscope.

As previously mentioned, the probe assembly 110 extends distally through the outer housing 108. In some examples, the distal end portion 152 may include a flared distal passage 177 (FIG. 6) having a trumpet-like shape configured (e.g., sized and shaped) to help reduce stress on the distal supply line 161 and/or the inner sheath 114 and the outer sheath 112, such as during manipulation of the outer housing 108 while the probe assembly 110 is within an endoscope, by progressively engaging the probe assembly 110 to limit its axial deflection and/or bending to a gradual curvature. Such an arrangement may help to prevent kinking or breakage of the tube 107 and the distal supply line 161 passing there though.

The outer sheath 112 may also, in some examples, pass through, and be bonded within, a sleeve 113 fixedly secured to the distal end portion 152 in a location proximal to the flared distal passage 177. In such examples, the sleeve 113 may increase the deflection resistance of the probe assembly 110 near the outer housing 108 to further reduce the chance of kinking and/or breakage of the outer sheath 112, inner sheath 114, or the distal supply line 161. The sleeve 113 may be made from, for example, but not limited to, polyolefin heat shrinkable tubing, among others heat shrinkable materials, polymeric materials such as, but not limited to, polyurethane, or elastomeric materials, such as, but not limited to, silicone.

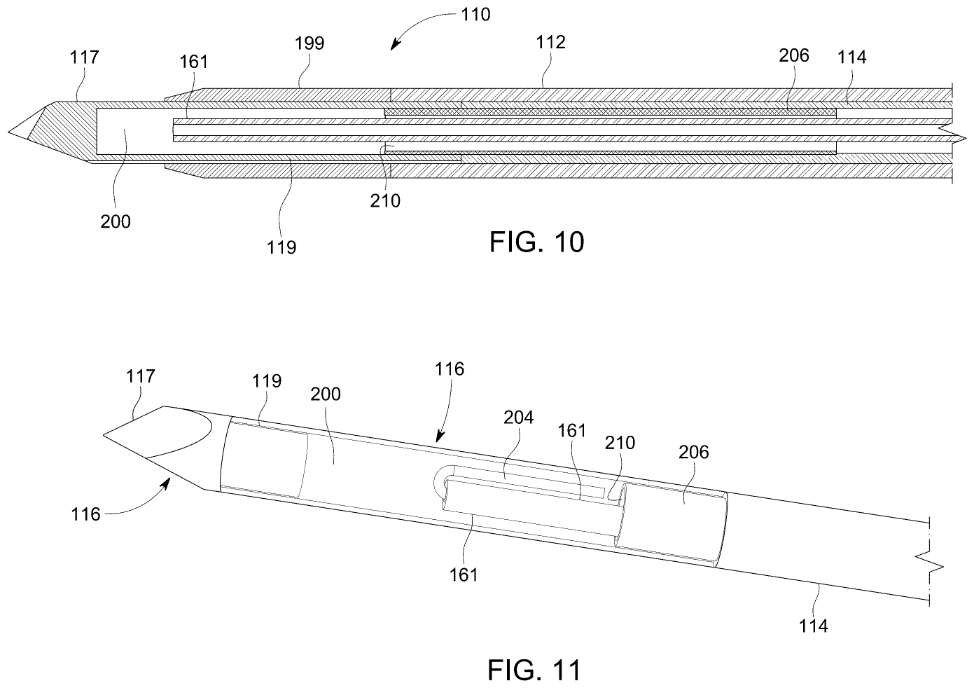

In other examples, the outer sheath 112 may be bonded or otherwise fixedly secured directly to the distal end portion 152 of the outer housing 108 to prevent the outer sheath 112 from moving relative to the inner sheath 114. In some examples, the outer sheath 112 may be formed from a flexible material, such as including, but not limited to, a braided composite of polyether block amides (e.g., “PEBAX”) to provide the outer sheath 112 with a rubber-like or elastic characteristics. In some examples, the outer sheath 112 may further include a distal cap 199 (FIGS. 10-11) defining a frustoconical, or tapered, shape that may form a gradual taper between an outer diameter of the outer sheath 112 and an outer diameter of the inner sheath 114.

In some examples, the outer sheath 112 may further be comprised of an inner layer and an outer layer. In such examples, the inner layer may be a liner layer made from a material that is more lubricious than the material comprising the outer layer in order to help reduce the force that may be used to translate the inner sheath 114 within the outer sheath 112. In some such examples, such a lubricous material may be, but not limited to, polyether block amides (e.g., “PEBAX”) with a polyimide additive, etched fluorinated ethylene propylene, etched polytetrafluoroethylene, or high-density polyethylene.

In some examples, the distal cap 199 may also increase the axial or lateral stiffness at a distal terminus of the outer sheath 112, such as to help the distal tip 116 penetrate tougher tissue. Accordingly, in such examples, the distal cap 199 may be made from a stiffer material than other portions of the outer sheath 112. The distal cap 199 may be made from various materials, such as including, but not limited to, a braided composition of polyether block amides (e.g., “PEBAX”) having a stiffening filler comprising up to 60 precent by weight of said composition. In various examples, such a filler may be, but not limited to, talc, glass fibers, or carbon black, among others. In some examples, such a filler may also include radiopaque additives to improve visualization during fluoroscopic imaging, such as, but not limited to, tungsten or barium sulfate.

The distal cap 199 may also define a longitudinal length, as measured in a direction parallel to the longitudinal axis A1, such as, but not limited to, within an inclusive range of about 1 millimeter and about 10 millimeters. In one specific example, the distal cap 199 may also define a longitudinal length of about 3 millimeters.

As previously mentioned, the outer sheath 112 may define a longitudinal length measured in direction parallel to the longitudinal axis A1, and inner and outer diameters, that may be varied widely to configure the sampling system 106 for various surgical procedures or surgical scopes. However, several non-limiting ranges of example dimensions are provided below. In some examples, the outer sheath 112 may define an overall length with an inclusive range of about 100 centimeters and about 130 centimeters. In one specific example, the outer sheath 112 may define a longitudinal length of about 115 centimeters. In some examples, the outer sheath 112 may also define an outer diameter within an inclusive range of about 1 millimeter and about 2.5 millimeters. In one specific example, the outer sheath 112 may define an outer diameter of 1.3 millimeters.

In some examples, the outer sheath 112 may define an inner diameter within an inclusive range of about 0.7 millimeters and about 1 millimeter. In one specific example, the outer sheath 112 may define an inner diameter of about 0.75 millimeters. In view of the above, the probe assembly 110 may have a substantially smaller outer or maximum diameter than any presently existing cryobiopsy device or cryoablation device, which may enable access to many anatomical locations previously inaccessible to such devices, such as deep or peripheral lung nodules, and reduce the risk of tissue damage and/or post operative bleeding. Moreover, such configurations may allow for a higher tissue sampling capacity and/or payload. For example, if the distal tip 116 or the outer sheath 112 defines an outer diameter of about 0.5 millimeters and is positioned within a traditional endoscope having a working channel of about 2 millimeters, about 94 percent of a diameter of the area within the working channel may be available for tissue samples to be retracted through or other instruments to pass.

Generally, the outer sheath 112 may be provided to protect surrounding tissue from both thermal damage (e.g., through undesirable cooling and/or freezing) and, in some examples, sharp surfaces or edges of the distal tip 116. The inner sheath 114 may be configured to be freely translatable with the outer sheath 112. In some examples, the inner sheath 114 may define an outer diameter measuring within an inclusive range of about 0.001 inches and about 0.003 inches less than the inner diameter of outer sheath 112. In one specific example, the inner sheath 114 may define an outer diameter of about 0.0017 inches less than the inner diameter of the outer sheath 112. The inner sheath 114 may also define various inner diameters, such as within an inclusive range of about 0.015 inches to about 0.030 inches. In one specific example, the inner sheath 114 may define an inner diameter of about 0.021 or 0.025 inches.

The inner sheath 114 may define a somewhat similar longitudinal length, as measured in a direction parallel to the longitudinal axis A1, to the outer sheath 112. In some examples, however, the inner sheath 114 may be slightly shorter. In some such examples, the inner sheath 114 may measure within an inclusive range of about 1 to about 10 centimeters shorter than the outer sheath. In one specific example, the inner sheath 114 may define a longitudinal length about 5 centimeters shorter than the outer sheath 112. Finally, the inner sheath 114 may be made from various materials, such as, but not limited to, TROGAMID®, polyamide, other polymeric materials, or a metallic material such as nitinol. In some examples, the inner sheath 114 may be significantly more flexible than the outer sheath 112, such as by lacking woven composition or a stiffening filler included in the outer sheath 112 and/or distal cap 199.

The distal tip 116 may be attached to, and extend distally from, a distal terminus of the inner sheath 114. In some examples, the distal tip 116 includes a first portion 117 (FIGS. 10-11) and a second portion 119 (FIGS. 10-11). In some examples, the first portion 117 and the second portion 119 may be integral with one another (e.g., a single component tip). In other examples, the first portion 117 and the second portion 119 may be formed from two separate pieces welded or otherwise bonded together (e.g., a two-component tip). In some examples, the first portion 117 may comprise a distal end portion or segment that is shaped for penetrated or otherwise contacting tissue to collect a biopsy sample, and the second portion 119 may extend proximally therefrom. In some examples, such as shown in FIGS. 10-11, the second portion 119 be hollow, such as by virtue of a defining a cavity 200, and the first portion 117 may be solid such that the cavity 200 is blocked or sealed off from a distal end surface of the first portion 117.

In other examples, the second portion 119 and the first portion 117 may be entirely solid, and the second portion 119 may define a recessed or reduced diameter outer surface relative to the first portion 117 to enable an outer diameter of the inner sheath 114 to extend flush with an outer diameter of the first portion 117. In still further examples, the second portion 119 and the first portion 117 may define a passage extending through to a distal end surface or terminus of the distal tip 116 so that the distal supply line 161 is instead in fluid communication with an exterior of the distal tip 116. Such a configuration may enable the probe assembly 110 to be configured for tissue ablation procedures, as the refrigerant will pass through both the distal supply line 161 and the distal tip 116 before expanding near or within targeted tissue.

The distal tip 116 may define various longitudinal lengths as measured in a direction parallel to the longitudinal axis A1. For example, the distal tip 116 may define a longitudinal length, as measured between a distal terminus of the first portion 117 and a proximal terminus of the second portion 119, within an inclusive range of about 3 millimeters and about 15 millimeters. In one specific example, the distal tip 116 may define a longitudinal length of about 10 millimeters. The distal tip 116 may also define a maximum outer diameter that is generally similar, or equivalent, to a maximum outer diameter of the inner sheath 114. For example, the distal tip 116 may define an outer diameter within an inclusive range of about 0.5 millimeters and about 1 millimeter. In one specific example, the distal tip 116 may define a maximum outer diameter of about 0.75 millimeters.

As previously noted, the second portion 119 of the distal tip 116 may be secured to a distal terminus of the inner sheath 114 such that an outer diameter of the inner sheath 114 extends, in a direction orthogonal to the longitudinal axis A1, flush with an outer diameter of distal tip 116. In some such examples, such as those as shown in FIGS. 10-11, 13, 15, and 17, a connecting element 206 may be bonded to, such as via welding, reflow or adhesive bonding, or other surface bonding techniques, an inner diameter of the second portion 119 and an inner diameter of the inner sheath 114. In such examples, a distal end surface of the inner sheath 114 (e.g., a surface extending orthogonal to the longitudinal axis A1) may also abut, and be bonded to, via reflow or adhesive bonding, among other surface bonding techniques, to a proximal end surface of the second portion 119 (e.g., a surface extending orthogonal to the longitudinal axis A1). In some examples, an outer diameter or surface of the distal supply line 161 may be welded, or otherwise bonded, to an inner diameter or surface of the connecting element 206 to help to increase the axial stiffness and tensile strength of the distal supply line 161.

In some examples, the connecting element 206 may be a tube defining an outer diameter selected to contact and engage an inner circumference or diameter of the second portion 119 and the inner sheath 114, respectively. In other examples, the connecting element 206 may be comprised of a plurality of axially oriented runners or stringers each distributed equidistantly around an inner circumference or diameter of the second portion 119 and the inner sheath 114, respectively. Finally, the connecting element 206 may be formed from a variety of materials including, but not limited to, stainless steel, titanium, or nitinol.

The connecting element 206 may help to strengthen the interface between the distal tip 116 and inner sheath 114, such as relative to a bond formed solely between the distal tip 116 and the inner sheath 114. Additionally, the connecting element 206 may serve to function as a reinforcing member that improves axial or lateral stiffness and/or reduces axial elongation (e.g., stretching) of the inner sheath 114 near the distal tip 116, such as caused by a user pulling distally on the outer housing 108 or the position controller 118, or the pressure generated by gas expansion. In additional examples, the outer sheath 112 and/or the inner sheath 114 may include additional bracing and/or reinforcing features to tailor their axial stiffness or lateral stiffness resistance, such as by including, among other features, a stacked coil, one or more metallic wires, or composite axial runners embedded therein, or alternately a laser-cut hypotube thereover or therein.

Finally, with regard to the axial and/or lateral stiffness of the inner sheath 114, the combination of a relatively dense material for the distal tip 116, such as, not limited to, stainless steel, may significantly contribute to the flexibility of a sampling or ablation probe, at least because the distal tip 116 is relatively heavy and the inner sheath 114 and the outer sheath 112 may be constructed from a relatively thin or small diameter tubular materials. In some such examples, this combination may help assist the probe assembly 110 navigate through highly tortious anatomy.

In some examples, the inner sheath 114 and/or the outer sheath 112 may also include radiopaque visualization markings, such as bands or patterns made from, but not limited to, tungsten or barium sulfate, spaced equidistantly or non-equidistantly along their longitudinal lengths. As is known in the art, such radiopaque bands or markings may act as depth indicators to show physician or clinician how deep the device is inside a patient, or how it is positioned relative to the endoscope, without the use of fluoroscopic imaging. Additionally, when the distal tip 116 of metallic construction, it may also inherently be radiopaque and may thus further aid a user in positioning the probe assembly 110 with a patient.

As previously noted, the first portion 117 of the distal tip 116 may engage tissue. In this regard, the first portion 117 may define a variety of different three-dimensional shapes, such as depending upon the density or other physical characteristics of tissue to be sampled and/or ablated, or the amount of tissue to be retrieved. In some examples, the shape of the first portion 117 may be similar to existing sharpened (e.g., cutting edged or needle pointed) probe tips, such as those for circumferential tissue acquisition. In other examples, the shape of the first portion 117 may be similar to existing semi-sharp (e.g., tapered, pyramidical, frustoconical, etc.) probe tips, such as those for tangential tissue acquisition. In additional examples, the shape of the first portion 117 may be similar to existing blunt (e.g., semi-hemispherical or flattened) probe tips, such as those for en face tissue acquisition.

In some specific examples, the first portion 117 may form a tantalum needle tip, or a trocar tip such as, but not limited to, a diamond trocar tip, a pyramidal trocar tip, a needle trocar tip, a dilating trocar tip, a conical pencil-like needle tip, or a lancet style tip. In further examples, the distal tip 116 may also be surface treated with a variety of biocompatible materials such as, but not limited to, gold, such as to increase radiopacity, or may further be textured and/or etched to improve visibility for an operating clinician.

Turning back to the distal supply line 161, the distal supply line 161 carries compressed gas, such as in a liquid state, from the filter 160 of the first axial tube valve assembly 154, or any other valve assembly disclosed herein, to the distal tip 116, and is housed within the inner sheath 114. In some examples, such as illustrated in FIGS. 10-11, 13, 15, and 17, a distal terminus of the distal supply line 161 may be located within the cavity 200 defined by the second portion 119 of the distal tip 116 so that compressed gas may exit the distal supply line 161 and expand (e.g., boil from a liquid state to a gaseous state) directly inside the distal tip 116 for a highly-efficient cooling effect. In this respect, the relatively large volume of the cavity 200, as compared to the relatively small inner diameter of the distal supply line 161, helps to facilitate rapid expansion of the refrigerant and thereby rapid cooling of the distal tip 116. However, the distal supply line 161 may alternatively terminate proximally of the distal tip 116, such as in examples that may include a solid distal tip 116.

Once the refrigerant transitions from a liquid state to a gaseous state within the distal tip 116, it begins moving in a proximal direction within the inner sheath 114 as gas continuously exits the distal supply line 161. In this regard, a size differential between an outer diameter of the distal supply line 161 and an inner diameter of the inner sheath 114 creates an exhaust lumen 210 for removing exhaust gases. In some examples, the size of the exhaust lumen 210 may be configured to achieve a desired amount of backpressure. In some examples, the exhaust lumen 210 may be radial space or distance, as measured orthogonally relative to the longitudinal axis A1, within an inclusive range of about 0.008 inches to about 0.013 inches.

The inner sheath 114 may define an exhaust lumen 210 due to a size differential between an outer diameter of the distal supply line 161 and an inner diameter of the inner sheath 114 and/or the connecting element 206 to enable expanded gases (e.g., exhaust gases) to leave the probe assembly 110 in a proximal direction. The size of the exhaust lumen 210 may be selected based on a desired amount of exhaust backpressure, which, as previously noted, may at least partially dictate the maximum size of an ice ball created in tissue at or near the distal tip 116 as a result of varying the boiling point of the refrigerant. For example, reducing a diametric gap between an outer diameter of the distal supply line 161 and an inner diameter of the inner sheath 114 to increase exhaust backpressure may cause an increase the boiling point of the refrigerant and thus reduce a maximum size of the ice ball created in tissue near, or in contact with, the distal tip 316.

Conversely, increasing a diametric gap between an outer diameter of the distal supply line 161 and an inner diameter of the inner sheath 114 and/or the connecting element 206 to decrease exhaust backpressure may reduce the boiling point of the refrigerant and thus increase a maximum size of the ice ball created in tissue near, or in contact with, the distal tip 316. Additionally, an amount of exhaust backpressure may, as those skilled in the art will appreciate, help to maintain the refrigerant under pressure throughout the entire refrigerant flow path (e.g., the distal supply line 161, a valve assembly, the proximal supply line 140, the connecting member 138, the piercing element 136, and the refrigerant cartridge 126 of the refrigerant system 104) until it exits the distal supply line 161, which may help to prevent a phase change from a liquid to a gas.

The inventors have also recognized that the exhaust backpressure may be selected or configured based on, but not limited to, the physical characteristics of the tissue to be sampled, the desired tissue sample size, and/or environmental factors such as ambient heat or natural ground elevation (e.g., height above sea level) that may affect the natural flow rate of exhaust gases.

In some examples, the distal supply line 161 may include a flow regulator 204 (FIGS. 10 and 13). The flow regulator 204 may be a structure that may limit or control the refrigerant flow rate through the distal supply line 161 in the distal tip 116, as well as also help ensure the refrigerant is maintained under a desired or otherwise adequate pressure at least until it exits the distal supply line 161. In some examples, such as shown in FIGS. 10-11, the flow regulator 204 may be a solid wire placed within a distal end or portion of the distal supply line 161. In some such examples, the flow regulator may terminate at, or extend slightly beyond, a distal terminus of the distal supply line 161. In other examples, the flow regulator 204 may be bent about 180 degrees around the distal terminus of the distal supply line 161 so that it extends into the exhaust lumen 210, which may further increase exhaust backpressure within the refrigerant flow path by creating a bottleneck effect at a proximal entrance to the exhaust lumen 210.

Accordingly, it is to be appreciated that both the longitudinal length, as measured in a direction parallel to the longitudinal axis A1, and the outer diameter, of the flow regulator 204 may be selected to vary the refrigerant flow rate through the distal supply line 161, the supply backpressure of the refrigerant (e.g., within the distal supply line 161), and the exhaust backpressure of the refrigerant (e.g., after exiting the distal supply line 161). In some examples, the flow regulator 204 may define an outer diameter within an inclusive range of about 0.002 and 0.005 inches. In one specific example, the flow regulator 204 may define an outer diameter of about 0.004 inches. In another specific example, the difference between an outer diameter of the flow regulator 204 and an inner diameter of the distal supply line 161 may be about 0.002 inches. In some examples, the flow regulator 204 may define a longitudinal length measuring within an inclusive range of about 0.5 inches and about 5 inches. In some specific examples, the flow regulator 204 may define a longitudinal length of about 1 inch or about 4 inches.

In some examples, an effective outlet diameter (e.g., the size differential between an outer diameter of the flow regulator 204 and an inner diameter of the distal supply line 161) to length (e.g., a length of a portion of the flow regulator 204 within the distal supply line 161) ratio may be within an inclusive range of about to about 0.2 to about 0.0004 inches. The flow regulator 204 may also be made from various materials, such as, but not limited to, stainless steel, titanium, coated steel, or a polymeric material such as polyamide or polyether ether ketone, among others. In some alternative examples, a distal end of the distal supply line 161 may be pinched, tapered, or otherwise narrowed to regulate or otherwise restrict the flow rate of refrigerant exiting from the distal supply line 161. In further examples, the flow regulator 204, or the distal supply line 161, may include features such as laser-cut holes and/or slits to vary flow characteristics or backpressure in area or region near a distal end of the distal supply line 161.

The inventors have also appreciated that the inclusion of the flow regulator 204 may, in some examples, be dependent upon the physical characteristics of the refrigerant. For example, relative to liquid nitrous oxide, liquid carbon dioxide, must be maintained at higher pressures in order to prevent it solidifying, and as such, an increased amount of backpressure in the refrigerant flow path must be maintained. In such examples, the flow regulator 204 may be employed. Conversely, as liquid nitrous oxide may be maintained at a lower pressure than liquid carbon dioxide without transitioning from a liquid to a gas, the flow regulator 204 may, in some examples, be unnecessary because adequate backpressure may be obtained, for example, through the use of a relatively small diameter refrigerant flow path.

As best shown in FIG. 9, a proximal terminus of the inner sheath 114 and the exhaust lumen 210 defined thereby is located within a fluid connector 170, illustrated in shadow in FIG. 10 to reveal an exhaust passage 171 therethrough, which is suspended within the position controller 118. Generally, the fluid connector 170 may be a structure or a component assembly that may fluidly connect the exhaust lumen 210 to an exhaust line 141. For example, a proximal terminus of the inner sheath 114 may be bonded, such as via reflowing, laser welding, or adhesive bonding, among other surface bonding techniques, within a first recess 169 (FIG. 9) of the fluid connector 170, and a distal terminus of the exhaust line 141 may be bonded, such as via reflowing, laser welding, or adhesive bonding, among other surface bonding techniques, within a second recess 173 (FIG. 9) of the fluid connector 170; and, the first recess 169 and the second recess 173 are in fluid communication with the exhaust passage 171.

In view of the above, exhaust gases flowing proximally from the distal tip 116 through exhaust lumen 210 may pass through the exhaust passage 171, as indicated by arrow 215 (FIG. 9) and into the exhaust line 141. Finally, the distal supply line 161 also extends distally, through the fluid connector 170 from its proximal terminus at the filter 160, and into the inner sheath 114 in direction to, or concentric with, the longitudinal axis A1. In this respect, the fluid connector 170 may be potted with an adhesive or other sealing material at the location where the distal supply line 161 enters the first recess 169 to prevent leakage of exhaust gases into the outer housing 108 and/or the position controller 118.

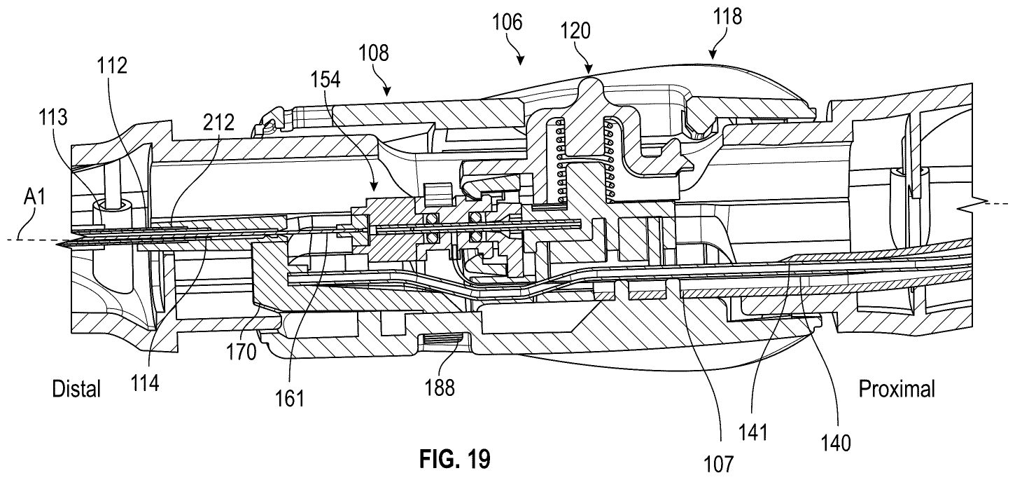

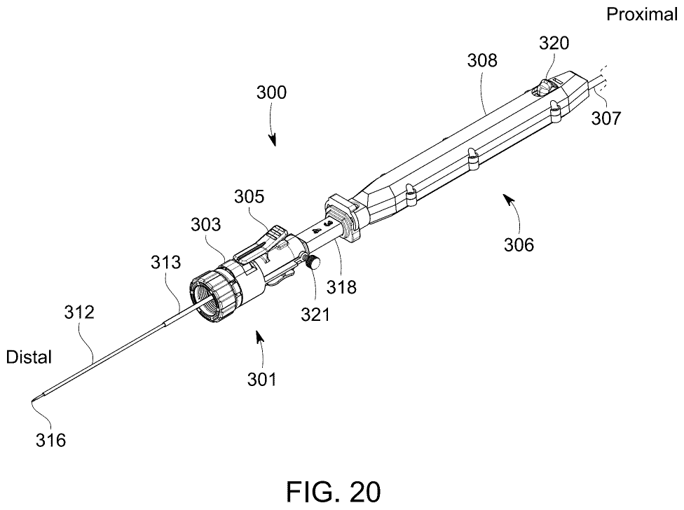

As best shown in FIGS. 19-20, the exhaust line 141 continues proximally from the fluid connector 170 to a proximal terminus within the tube 107, thus enabling exhaust gases to travel proximally around the proximal supply line 140 therein and vent to the atmosphere through the exhaust port 144, as previously discussed with reference to the base housing 124 (FIGS. 1-4). In this regard, the tube 107 may be potted with an adhesive or sealing material at the location where the exhaust line 141 enters the tube 107 to thereby prevent any leakage of the exhaust gases into the outer housing 108 and/or the position controller 118. In some examples, the exhaust line 141 may define a longitudinal length, as measured in a direction parallel to the longitudinal axis A1, within an inclusive range of about 1 inch and about 15 inches. In more specific examples, the exhaust line 141 may define a longitudinal length measuring about 10 inches.

In some examples, the exhaust line 141 may be configured to enable a user to selectively increase exhaust backpressure to, in turn, help limit or control the size of an ice ball created in tissue at or near the distal tip 116, as the inventors have recognized that increasing the exhaust backpressure may increase the boiling point of the refrigerant and thus reduce a maximum size of the ice ball created in tissue. In various examples, the exhaust backpressure may be selected or otherwise configured based on the physical characteristics of the tissue to be sampled, the desired tissue sample size, and/or environmental factors such as ambient heat or natural ground elevation (e.g., height above sea level) that may affect the natural flow rate of exhaust gases.

In some such examples, the exhaust line 141 may be made from a deformable material and the outer housing 108 may include or contain a pivotable, slidable, rotatable, or otherwise movable element configured to enable a user to impart a crush force to the exhaust line 141 to thereby reduce its internal diameter and increase exhaust backpressure by restricting or limiting the flow rate of exhaust gases proximally through the exhaust line 141. In other examples, the outer housing 108 may include or contain a rotatable mechanism adapted to enable a user to rotate a plurality of differently sized apertures or nozzles into the exhaust line 141 to thereby selectively restrict or limit the flow rate of exhaust gases proximally through the exhaust line 141 and into the tube 107.

Turning now to the position controller 118, as previously mentioned, the position controller 118 may be translatable about the outer housing 108 to extend and retract the inner sheath 114 and the distal tip 116 within the outer sheath 112. In some examples, such as shown in FIG. 6, the position controller 118 may be a two-piece assembly comprised of a first portion 250 and a second portion 252. As also previously mentioned, the fluid connector 170 is suspended within the position controller 118, and the fluid connector 170 retains a proximal terminus of the inner sheath 114. For example, the position controller 118 may receive a base 188 (FIGS. 6 and 18-19) of the fluid connector 170, such as by defining an aperture 168 (FIG. 6) and/or other geometry configured to interface with base 188 to prevent relative movement between the position controller 118 and the fluid connector 170. As such, the position controller 118 and the fluid connector 170 may be moved together to cause a corresponding movement in the inner sheath 114 and the distal tip 116.

Notably, in this regard, the fluid connector 170 is not connected to the outer sheath 112, which, as previously mentioned above, is affixed either to the outer housing 108, or in some examples, to the sleeve 113. For example, the fluid connector 170 may define a bore 212 (FIG. 19) configured (e.g., sized and shaped) to provide clearance for the outer sheath 112 so that the fluid connector 170 may translate freely around the stationary outer sheath 112. Further, in this regard, as the first axial tube valve assembly 154 and the refrigerant controller 120, and each of their individual components, are suspended with the position controller 118, any movement of the position controller 118 will result in corresponding movement of the first axial tube valve assembly 154 and the refrigerant controller 120, among other components such as the proximal supply line 140 and the tube 107, without changing the relative positions of the first axial tube valve assembly 154 and the refrigerant controller 120 with respect to each other.

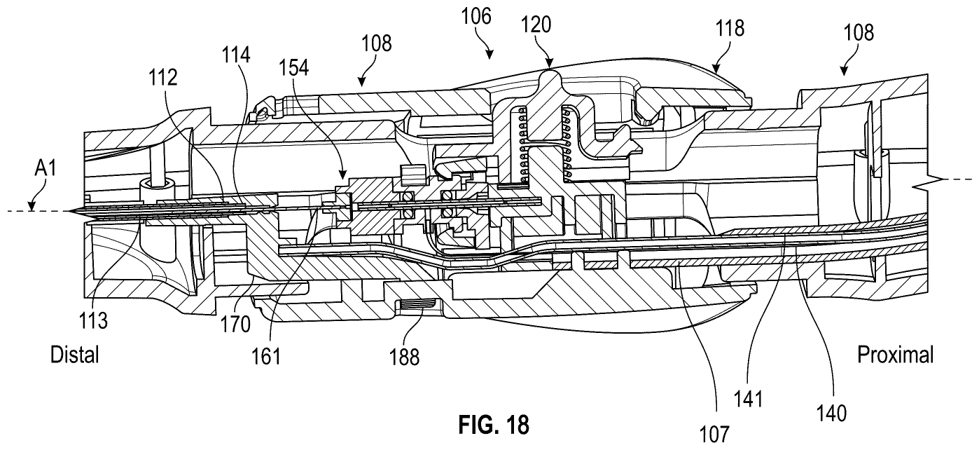

In view of all the above, the position controller 118 may be selectively translated into a relatively proximal position, such as shown in FIG. 19, to cause the distal tip 116 to retract into the outer sheath 112, such as shown in FIG. 13, and translated in a relatively distal position, such as shown in FIG. 18, to cause the distal tip 116 extend beyond the outer sheath 112, such as shown in FIG. 15. In various examples, the outer housing 108 and/or the position controller 118 may be configured to allow the position controller 118 to have a total longitudinal travel, in a direction parallel to the longitudinal axis A1, within an inclusive range of about 1 millimeter to about 10 millimeters.

The size of the tissue sample collected via cryo-adhesion (e.g., after cooling of the distal tip 116 and freezing tissue in contact therewith) may depend upon the distance the distal tip 116 protrudes beyond the outer sheath 112. For example, if a smaller tissue sample is desired, a user may translate the position controller 118 distally to cause the distal tip 116 to extend about 1-3 millimeters beyond the outer sheath 112. Alternatively, if a larger sample is desired, a user may translate the position controller 118 distally to cause the distal tip 116 to extend about 4-8 centimeters beyond the outer sheath 112.

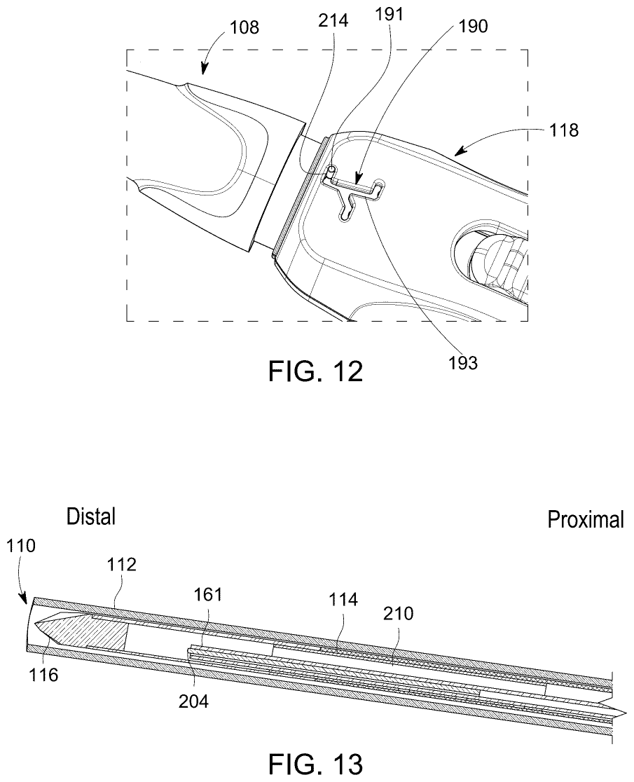

In some examples, the position controller 118 may further prevent inadvertent extension or retraction of the distal tip 116 during a surgical procedure. For example, as shown in FIGS. 5, 12, 14, and 16, the position controller 118 may define a gate 190 that may receive a locking element 191. The locking element 191 may be a pin or another type of projection or protrusion fixedly extending outwardly from an outer surface of the outer housing 108. The gate 190 may be patterned or shaped guide path for dictating the motion(s) and/or manipulations required through which the position controller 118 must be moved in order extend or retract the distal tip. In some examples, the gate 190 may define a central path 193 (FIGS. 12, 14, and 16) which extends parallel to the longitudinal axis A1, or otherwise in a generally longitudinally direction, and a plurality of offset paths each deviating from the central path 192 at an angle, such as within an inclusive range of about 45 degrees and about 90 degrees relative to the longitudinal axis A1 and/or the central path 193.

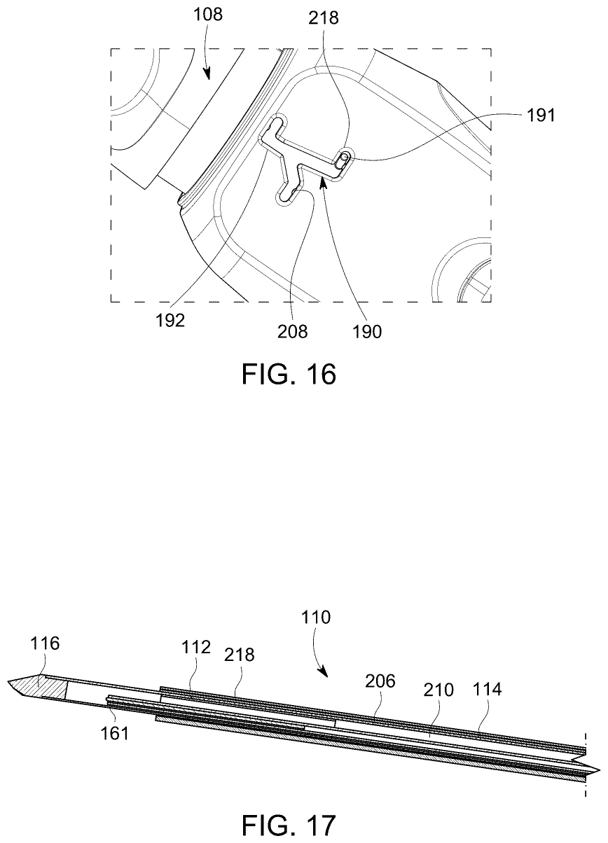

In such configurations, the position controller 118 may also rotate about the outer housing 108 and/or the longitudinal axis A1 to move the locking element 191 from the central path 192 into the each of the plurality of offset paths. Each of the plurality of offset paths may be spaced axially apart, such relative to the longitudinal axis A1, to thereby create a different pre-defined longitudinal position for the distal tip 116 relative to the outer sheath 112. In this regard, the plurality of offset paths may include one, two, three, four, five, six, or other numbers of individual paths to enable a user to easily select between a corresponding number of pre-defined longitudinal positions for the distal tip 116. Further, in some examples, any, or all, of the plurality of offset paths may include a protrusion 208 (FIG. 16) sized and shaped to increase resistance to the locking element 191 by contacting the locking element 191 as it passes into, or exits from, an individual offset path.

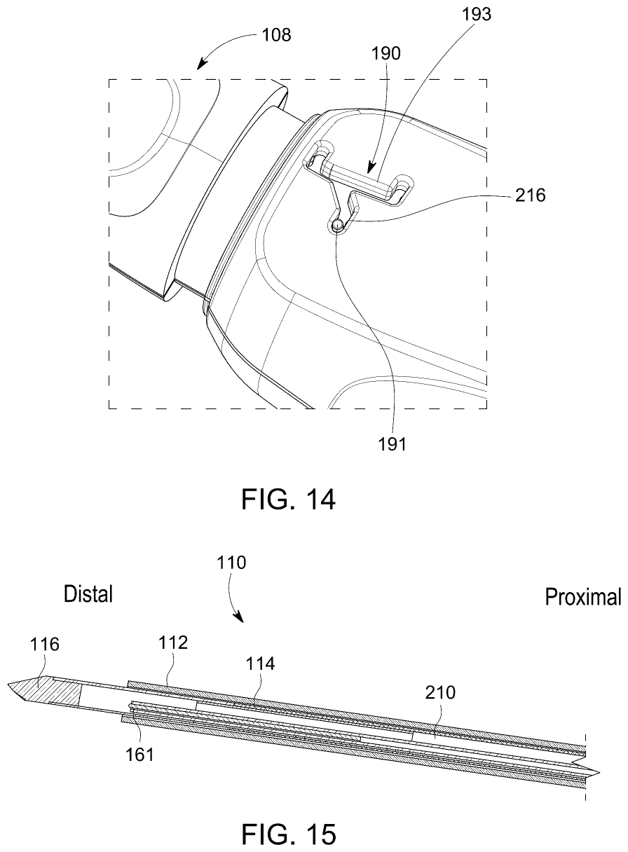

In some examples, such as shown in FIGS. 12, 14, and 16, the plurality of offset paths may include a first path 214 (FIG. 12) so that the position controller 118 has a first position, a second path 216 (FIG. 14) so that the position controller 118 has a second position, and a third path 218 (FIG. 16) so that the position controller 118 has a third position (FIG. 16). In some such examples, the distal tip 116 may be in a fully retracted position, as shown in FIG. 13, when the position controller 118 is in its first position (FIG. 12), the distal tip 116 may be a partially extended position, as shown in FIG. 15, when the position controller is in its second position (FIG. 14), and the distal tip 116 may be positioned in a fully extended position, as shown in FIG. 17, when position controller 118 is in its third position (FIG. 16). In one specific example, the distal tip 116 may extend about 3 centimeters distally beyond the outer sheath 112 when the position controller 118 is in its second position and the distal tip 116 may extend about 6 centimeters distally beyond the outer sheath 112 when the position controller its third position.

In view of the above, at least by virtue of requiring both longitudinal translation and axial rotation, the distal tip 116 may be securely maintained in different longitudinal positions, such as to prevent accidently movement and resist axial forces generating by advancing the distal tip 116 into tissue. However, the position controller 118 may be retained in various axial positions to resist axial forces using a variety of other mechanical techniques or devices known in the art, such as, among others, a ratcheting or toothed mechanism between the position controller 118 and the outer housing 108, a pin-and-hole detent mechanism between the position controller 118 and the outer housing 108, or a magnetic latching and/or braking mechanism between the position controller 118 and the outer housing 108.

The position controller 118, the outer housing 108, the refrigerant controller 120, among other components received therein, may also be comprised of various materials, such as including, but not limited to, polymeric materials such as a acrylonitrile butadiene styrene (“ABS”), an acrylonitrile butadiene styrene and polycarbonate blend (e.g., “Bayblend”), polycarbonate, nylon (polyamide), or high impact polystyrene (“HIPS”), among others. In further examples, the outer housing 108 could also include a gauge and/or sensor in communication with distal tip 116 to visibly display to a user a temperature of the distal tip 116.

In an example method of performing a surgical procedure with the cryobiopsy device 100, a user may first open a sterile package containing a sterile portion housing the sampling system 106 and the refrigerant system 104. In one example, the refrigerant cartridge 126 and the cartridge nest 122 may be supplied to the user within a non-sterile part of the sterile package. Next, the user may insert the cartridge nest 122, with the refrigerant cartridge 126 contained therein, into the base housing 124 to establish fluid communication between the refrigerant system 104 and the sampling system 106. The user may then guide the endoscope 102, or another type of surgical scope, to a target anatomical location, such as by inserting the endoscope 102 into a patient through a natural body orifice. Subsequently, the user may advance the probe assembly 110 distally beyond a distal end of the endoscope 102, such as by moving the outer housing 108, to a location adjacent the tissue to be biopsied, ablated, or otherwise treated.

The user may then translate and/or rotate the position controller 118 to cause the distal tip 116 to emerge from the outer sheath 112 and into contact with tissue. In this regard, in some examples, prior to movement of the distal tip 116 into contact with tissue, the outer sheath 112 may first be pressed against the target tissue before the distal tip 116 is advanced therefrom, which may help to stabilize the probe assembly 110 on the target tissue and help reduce tissue mobility and/or deflection of the distal tip 116 away from the target tissue. Next, the user may translate and/or depress the refrigerant controller 120 to open a valve assembly within the sampling system 106, thus allowing refrigerant from the refrigerant system 104 to begin rapidly cooling the distal tip 116, and/or or begin dispersing gas directly into tissue, to ablate or adhere tissue to the distal tip 116. In some examples, the refrigerant controller 120 may be locked into an open position without the continuous application of pressure thereon. In some examples, the refrigerant controller 120 may be left in an open position within an inclusive range of about 3 seconds to about 2 minutes to adequately treat and/or bond tissue to the distal tip 116.

The probe assembly 110 may then be withdrawn proximally through the working channel 103 of the endoscope 102. In this regard, in some examples where the probe assembly 110 may be sized and shaped to have a sufficient air gap between the outer sheath 112 and the inner sheath 114, a tissue sample adhered to the distal tip 116 may be removed by advancing the outer sheath 112 distally over the distal tip 116, or by proximally retracting the distal tip 116 back into the outer sheath 112, before pulling the outer housing 108 in a proximal direction. In such examples, the ice ball and/or frozen tissue sample retained between the distal tip 116 and the outer sheath 112 may also significantly stiffen an otherwise highly flexible distal end region of the probe assembly 110, such as to help improve the ease at which the probe assembly 110 may be retracted into working channel 103. Finally, in some examples, the user may scrape or otherwise remove a collected tissue sample from distal tip 316. In this regard, the sterile package in which the cryobiopsy device 300 is supplied to the user may also, in some examples, include a disposable tissue removal device, such as a tissue scraper for aiding the user in separating tissue from the distal tip 316.

With regard the above, the inventors have also appreciated that a similar method may be performed to ablate or otherwise free and/or cool tissue to address a wide variety of medical conditions, such as, but not limited to, those set out further below. For example, if the distal tip 316 is instead configured to be completely hollow, or may otherwise define a passage or bore extending through distal end surface of the distal tip 316, refrigerant may flow distally therebeyond and into contact with tissue when the refrigerant controller 320 is in a fully open or partially open position.

Finally, in the example method described above, some, or all, of the steps may be performed through the use of a surgical robot remotely controlled by a physician or clinician to, for example, more precisely move the endoscope 102, the sampling system 106, and their relative positions with respect to each other which may be helpful in surgical procedures having a target location that is relatively deep within a patient and/or that is located within relatively tortious anatomy.

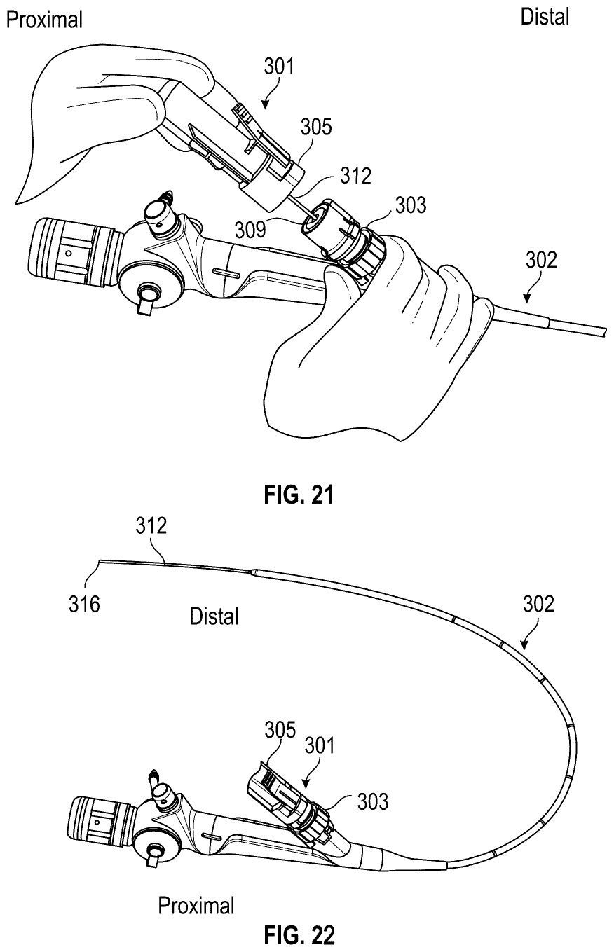

FIG. 20 is a perspective view of a cryobiopsy device 300 including a scope adapter 301, and FIGS. 21-22 illustrate the scope adapter 301 engaging with an endoscope 302. FIGS. 20-22 are discussed below concurrently. In some examples, the endoscope 302 may represent a variety of existing scopes defining a working channel defining a diameter within an inclusive range of about 1 millimeter and about 3 millimeters, and a longitudinal length within an inclusive range of about 60 centimeters and about 90 centimeters.

Like the cryobiopsy device 100 shown in, and described in detail with reference to, FIGS. 1-19 and 54 above, the cryobiopsy device 300 may be a sterile, single-use (e.g., disposable) tissue sampling or ablation system that may be used in interventional procedures. However, unlike the cryobiopsy device 100, the cryobiopsy device 300 may be used in surgical procedures where securing the sampling or ablation device to a surgical scope may be desirable or necessary, such as in hand-guided procedures to help prevent unintentional or undesirable (e.g., overly aggressive) movements therebetween.

The cryobiopsy device 300 may be comprised of three sub-assemblies, the refrigerant system 104 shown in, and previously described in detail with respect to FIGS. 1-4 above, a sampling system 306, and the scope adapter 301. As in the cryobiopsy device 100, the refrigerant system 104 is in fluid communication with the sampling system 306 by way of a tube 307 housing a proximal supply line and an exhaust lumen. Additionally, like the cryobiopsy device 100, the sampling system 306 includes an outer housing 308 forming a handle-shaped structure for a user to hold, and a probe assembly 310 which extends distally therefrom and may pass through a working channel of the endoscope 302. The probe assembly 310 may also be similar to the probe assembly 110 of the cryobiopsy device 100, at least in that it includes a distal tip 316 extendable from an outer sheath 213 to sample or ablate tissue.

However, in contrast to the sampling system 106, which utilizes a movable position controller (e.g., the position controller 118) to adjust the distal tip 116 relative to the outer sheath 112, the outer housing 308 of the sampling system 306 may be itself translatable proximally and distally to retract and advance the distal tip 316. More specifically, the outer housing 308 is fixedly connected to the outer sheath 312, and the outer housing 308 is translatable about a central body 318 extending between the outer housing 308 and the scope adapter 301.

Additionally, in contrast to the sampling system 106, the sampling system 306 includes the scope adapter 301. The scope adapter 301 may include any structure or component assembly comprising a connection mechanism for removably securing the central body 318 to a commercially available endoscope, such as an ultrasound endoscope, or another type of surgical scope, using mechanical coupling techniques, such as a threaded interface, a friction fit interface (e.g., snap fit), or a magnetic interface, among others.

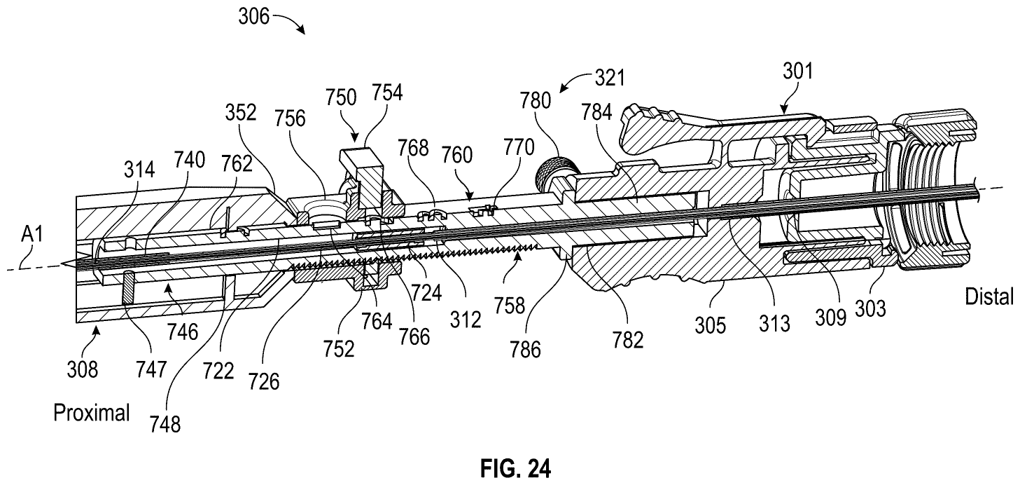

In some examples, the scope adapter 301 may be a two-piece structure comprised of a first component 303 that may interface with the endoscope 302 and a second component 305 that may interface with the central body 318. In such examples, the first component 303 may include a thread that mates with threads on the endoscope 302 in a manner consistent with many various styles of surgical scopes and scope adapters known in the art. In such examples, and as also known in the art, the first component 303 may define a probe opening 309 (FIGS. 21 and 24) that may enable a cryoprobe, such as the probe assembly 310, to be inserted into or retracted from the working channel of the endoscope 302. In some specific non-limiting examples, the first component 303 may be adapted to connect the second component 305 to a surgical scope from Fuji Film Holdings or Pentax Medical of Tokyo, Japan.

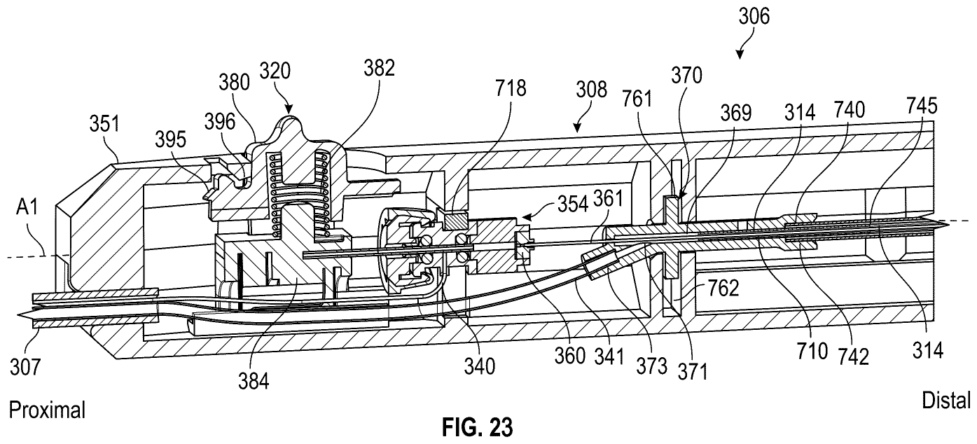

In some examples, the first component 303 may detachably engage the second component 305, such as, but not limited to, via a snap fit, a latch or clip mechanism, magnetic engagement, or other quick release mechanisms. By way of example, FIG. 22 shows the first component 303 secured to the endoscope 302 but detached from the second component 305, and FIG. 23 illustrates the first component 303 secured to the endoscope 302 and attached to the second component 305. In one example, such as illustrated in FIGS. 21-23, the scope adapter 301 may be the scope adapter included in the Compass Steerable Needle available from Serpex Medical of Santa Clara, California. In another example, the scope adapter 301 may be scope adapter included in the Recon Steerable Sheath available from Serpex Medical of Santa Clara, California. In a further example, the scope adapter 301 may be modified from the Compass Steerable Needle adapter or the Recon Steerable Sheath adapter to include an adjustment mechanism 321 (FIG. 21) adapted to allow adjustment of the outer sheath 312 of the probe assembly 310 with respect to the working channel of endoscope 302, even when the first component 303 is attached to the second component 305. Thus, the scope adapter 301 may enable the sampling system 306 to be used with a wide variety of different commercially available surgical scopes to obviate the need for a new, or otherwise customized, scope device.

In view of all the above, the cryobiopsy device 300, like the cryobiopsy device 100, does not require a conventional computerized refrigerant supply and/or control console to perform an endoscopic biopsy and/or ablation procedure. Accordingly, the cryobiopsy device 300 may be more portable, less expensive, and more accessible to both clinicians and patients than existing devices. The cryobiopsy device 300 is described in greater detail below with reference to FIGS. 23-32.

FIGS. 23-32 illustrate various views of the cryobiopsy device 300. FIGS. 23 and 24 also show a longitudinal axis A1 of the sampling system 306. FIGS. 23-32 are discussed below concurrently. As shown in FIGS. 23 and 32, the tube 307, with a proximal supply line 340 received therein, may pass through a proximal end portion 351 of the outer housing 308. The proximal supply line 340 may be similar or identical to the proximal supply line 140 previously discussed above.

In some examples, the proximal end portion 351 may include a flared proximal passage 375 (FIG. 32). The flared proximal passage 375 may be configured (e.g., sized and shaped) to help reduce stress on the proximal supply line 340 (FIG. 23) and/or the tube 307, such as during manipulation of sampling system 306 relative to the refrigerant system 104 (FIGS. 1-4), by progressively engaging the tube 307 to limit axial deflection and/or bending of the tube 307 to a gradual curvature. Such an arrangement may help to prevent kinking or breakage of the tube 307 and the proximal supply line 340 passing there though. In other examples, such as shown in FIG. 32, the tube 307 may simply extend through a correspondingly shaped aperture in the proximal end portion 351.

The refrigerant controller 320 may be configured, like the refrigerant controller 120 previously described in detail above, to enable a user to open and close any of various valve assemblies which may fluidly connect and disconnect the proximal supply line 340 and a distal supply line 361 (FIGS. 23 and 25-26). The refrigerant controller 320 and the distal supply line 161, including any of various components thereof, may be similar or identical to the distal supply line 161 previously described above. Additionally, as shown in FIG. 23, the outer housing 308 may include a first axial tube valve assembly 354 that may be identical to the first axial tube valve assembly 154 previously described above, except in that the first body portion 358 may be suspended in a mounting boss 718 of the outer housing 308 rather than the position controller 118. However, as with sampling system 106, the sampling system 306 may also utilize a wide variety of other valve assemblies with the outer housing 308, such as, but not limited to, any of the additional valve assemblies illustrated in, and described with reference to, FIGS. 33-53 below.

The refrigerant controller 320 may also, like the refrigerant controller 120, prevent inadvertent opening of the first axial tube valve assembly 354, among other valve assemblies, such as by including geometric features sized and shaped to engage corresponding geometric features of the outer housing 308. For example, the refrigerant controller 320 may include an activation button 380, a spring 382, and a sliding block 384 identical to the activation button 180, the spring 182, and the sliding block 184. As such, the activation button 380 may, in some examples, define a first angled surface 395 (FIG. 23) shaped to contact and engage a second angled surface 396 (FIG. 23) of the outer housing 308; and the activation button 380 may further define a third angled surface 397 (FIG. 32) shaped to contact and engage a fourth angled surface 398 (FIG. 32) of the outer housing 308. Each of the first angled surface 395, the second angled surface 396, the third angled surface 397, and the fourth angled surface 398 may function similarly to the first angled surface 195, the second angled surface 196, the third angled surface 197, and the fourth angled surface 198 previously described above.

Like the probe assembly 110 discussed in detail above, the probe assembly 310 may include the outer sheath 312, an inner sheath 314, and the distal tip 316. In some examples, the outer sheath 312, the inner sheath 314, and the distal tip 316 may be constructed from similar materials, define similar dimensions, and/or may include similar additional features, such as echogenic and/or radiopaque markings, as the outer sheath 112, the inner sheath 114, and the distal tip 116. Additionally, the outer sheath 312, the inner sheath 314, and the distal tip 316 may generally function similarly to the outer sheath 312, the inner sheath 314, and the distal tip 316, at least in that the outer sheath 312 receives the inner sheath 314, and the inner sheath 314 and its distal tip 316 are movable within the outer sheath 312.

In some examples, the inner and outer diameters of the outer sheath 312, the inner and outer diameters of the distal tip 316 and/or the inner and outer diameters of the inner sheath 314 (or its thermal jacket 315 discussed below) may also be similar or identical to the inner and outer diameters of the outer sheath 312, the inner and outer diameters of the distal tip 316, and/or the inner and outer diameters of the inner sheath 314. By way of example, outer sheath 312 may define an outer diameter within an inclusive range of about 1 millimeter and about 2.5 millimeters. In one specific example, the outer sheath 312 may define an outer diameter of 1.9 millimeters. Further, the outer sheath 312 may define an inner diameter within an inclusive range of about 1 millimeter and about 1.3 millimeters. In one specific example, the outer sheath 312 may define an inner diameter of 1.15 millimeters. However, the outer sheath 312, the inner sheath 314, and the thermal jacket 315 may be arranged differently and/or may be different in some other respects when compared to the outer sheath 112, the inner sheath 114, and the distal tip 116 of the sampling system 106, such as described below.

In this regard, the distal portion of the probe assembly 310, in contrast to the probe assembly 110, may additionally include a protective coil 332 (FIGS. 28 and 30) and a thermal jacket 315 (FIGS. 26 and 28). The protective coil 332 may be an individual component, or a component assembly such as a metallic element incorporated into a polymeric material layer, configured to help protect the endoscope 302 from the distal tip 316. In some examples, such as shown in FIG. 28, the protective coil 332 may be bonded to an inner diameter or circumference of the outer sheath 312. In some examples, the protective coil 332 may define a relatively short longitudinal length from its proximal terminus, shown in FIG. 28, to its distal terminus at or near the distal end of the outer sheath 112, such as shown in FIGS. 26 and 28, to help the outer sheath 312 maintain flexibility.

In some examples, the protective coil 332 may define a longitudinal length, as measured in a direction parallel to the longitudinal axis A1 (FIGS. 23 and 24), such as, but not limited to, within an inclusive range of about 0.75 inches and about 5 inches. In one specific example, the protective coil 332 may define a longitudinal length of about 2 inches. Finally, the protective coil 332 may be constructed, entirely or partly, from a variety of resilient materials such as, but not limited to, stainless steel, titanium, or coated or non-coated steel.

Further, as previously noted above, the probe assembly 310 may additionally include a thermal jacket 315 (FIGS. 26 and 28) disposed on the inner sheath 314. The thermal jacket 315 may be a material layer encompassing, and may be bonded to via laser welding, reflow, adhesive, or other surface bonding techniques, at least a portion of a longitudinal length of the inner sheath 314, such as defined in a direction parallel to the longitudinal axis A1 (FIGS. 23 and 24) of the sampling system 306. The thermal jacket 315 may extend from a location proximal, or proximally adjacent to, the distal tip 116 to a location beyond a proximal end or terminus of the protective coil 332, which may help to prevent inadvertent thermal damage to nearby tissue that could otherwise be in contact with a portion of the inner sheath 314. In this respect, the thermal jacket 315 may, in contrast to the inner sheath 114, be employed or otherwise include due to the inner sheath 114 being constructed from a metallic material including, but not limited to, stainless steel, nitinol, or titanium. In some examples, the thermal jacket 315 may also help to increase lubricity reduce friction between the protective coil 332 and the inner sheath 114.

In some examples, the thermal jacket 315 may, like the protective coil 332, define a relatively short longitudinal length in order to help the outer sheath 312 maintain a degree of flexibility. In some examples, the thermal jacket 315 may define a longitudinal length, as measured in a direction parallel to the longitudinal axis A1 (FIGS. 23 and 24), such as, but not limited to, within an inclusive range of about 4 centimeters and about 70 centimeters. In further such examples, the thermal jacket 315 may encompass a full or complete longitudinal length of the inner sheath 314, as measured in a direction parallel to the longitudinal axis A1. In one specific example, the thermal jacket 315 may define a longitudinal length of about 6 centimeters. In another specific example, the thermal jacket 315 may define a longitudinal length of about 70 centimeters.

The thermal jacket 315 may also be constructed from a variety of materials such as, but not limited to, high-density polyethylene, fluorinated ethylene propylene, or polytetrafluoroethylene, among others. In some examples, a distal end or terminus of the thermal jacket 315 may also comprise a tapered portion 399. The tapered portion 399 may define a frustoconical or tapered shape that may form a gradual taper between an outer diameter of the thermal jacket 315 and an outer diameter of the inner sheath 114 therein.

Additionally, the thermal jacket 315 may be positioned about the inner sheath 314 to leave a pre-determined longitudinal length of the distal tip 316 and/or the inner sheath 114 exposed for tissue sample collection via cryo-adhesion. For example, a distal end of the tapered portion 399 of the thermal jacket 315 may terminate within an includes range of about 1 centimeter and about 8 centimeters from a distal end surface of the distal tip 316. In some specific examples, a distal end of the tapered portion 399 of the thermal jacket 315 may terminate about 3 centimeters or about 6 centimeters from the from a distal end surface of the distal tip 316.

Further, the inner sheath 314 may be configured (e.g., sized and shaped) to be freely translatable with the outer sheath 312. For example, the thermal jacket 315 may define an outer diameter measuring within an inclusive range of about 0.001 inches and about 0.007 inches less than the inner diameter of outer sheath 112. In one specific example, the thermal jacket 315 may define an outer diameter of about 0.002 or about 0.003 inches less than the inner diameter of the outer sheath 312.

Finally, regarding the distal portion of the probe assembly 310, the distal tip 316 may, like the distal tip 116, be fixedly attached to, and extend distally from, a distal terminus or end portion of the inner sheath 314 as shown in FIGS. 26 and 28. Additionally, like the distal tip 116, the distal tip 316 may, in some examples, include a first portion 317 and a second portion 319. The first portion 317 may be similar, or identical, to any of the examples of the first portion 117 described in detail above. In some examples, such as shown in FIGS. 26 and 28, the second portion 319, like the second portion 119, be completely or partially hollow at least by virtue of defining a cavity 700 (FIGS. 26 and 28).

In such examples, the cavity 700 may function similarly to the cavity 200 of the distal tip 116, at least by providing additional space or volume for refrigerant exiting the distal supply line 361 to boil or otherwise expand into a gaseous state to enhance or otherwise improve cooling of the distal tip 316. Moreover, the distal supply line 161 may be similar or identical to the distal supply line 161 and may include similar additional features such as the flow regulator 204. In other examples, the second portion 319, like the second portion 119, may be solid. In one specific example, the distal tip 316 may define an outer diameter of about 0.8 millimeters. In other examples, the distal tip 316 may define an outer diameter within an inclusive range of about 0.5 millimeters and about 1 millimeter.

However, in contrast to at least the examples of the second portion 119 illustrated in FIGS. 10-11, the second portion 319 (FIGS. 26 and 28), may include a recessed outer surface 738 (FIG. 26) having a smaller outer circumference than an outer circumference of the first portion 317. In this respect, the recessed outer surface 738 may be sized and shaped to engage, and be bonded to, such as by laser welding, reflow, adhesive, or the other surface bonding techniques, an inner circumference 739 (FIG. 26) of the inner sheath 114 to thereby enable an outer circumference of the inner sheath 314 to be flush, as defined in a orthogonal to the longitudinal axis A1, with an outer circumference of the distal tip 116. Further, in such examples, the recessed outer surface 738 may be bonded, such as via laser welding, adhesives, or reflowing, among other surface bonding techniques, to an inner diameter or circumference of the inner sheath 114.

Finally, as previously noted above, the probe assembly 310 may, in some examples, be adapted for use in an endoscope, or other surgical scope, possessing a working channel defining a longitudinal length that is less than a longitudinal length of a working channel for which the probe assembly 110 is configured for use. In such examples, the inventors have recognized that, when the distal supply line 361 and the inner sheath 114, which define the exhaust lumen 311 therebetween, are reduced in longitudinal length, the flow rate of refrigerant through the distal supply line 161 and/or the flow rate of exhaust gases through the exhaust lumen 311 may increase. In view of the above, it is appreciated that such an increase in flow rate and/or backpressure may be compensated for, or otherwise addressed, by increasing the outer diameter and/or longitudinal length of the flow regulator 204.

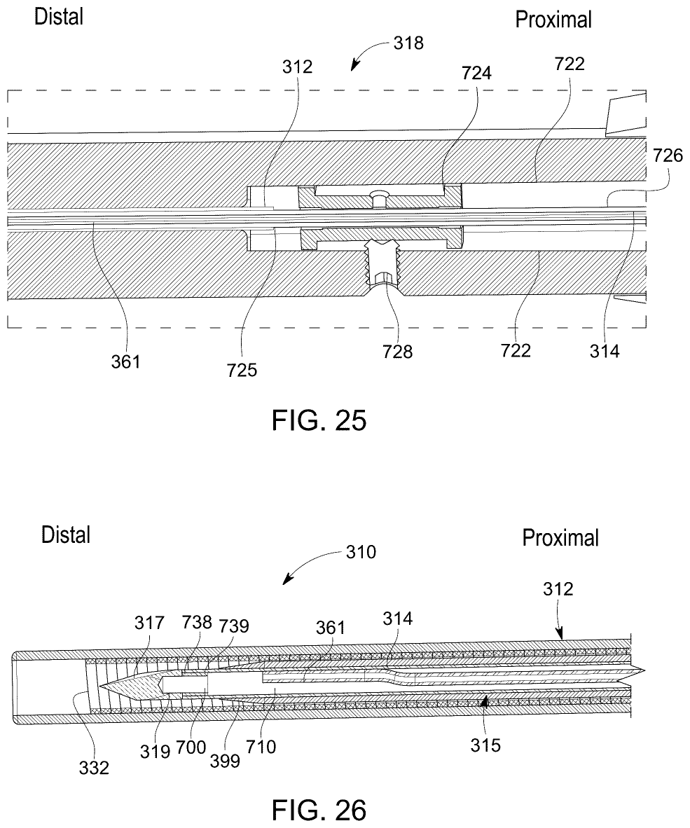

Turning back to the proximal portion of the probe assembly 310, the outer sheath 312 may extend distally to its distal end or terminus near the distal tip 316 from within the central body 318. In some examples, a proximal terminus 725 (FIG. 25) of the outer sheath 312 may be secured within a generally medial or middle portion of the central body 318, such as, but not limited to, within a proximal passage 722 (FIGS. 24 and 25) defined by a proximal portion of the central body 318. In such examples, the proximal passage 722 may be sized and shaped to accommodate a securing member 724 (FIGS. 24-25) comprising a separately formed component insertable distally into the central body 318 through the proximal passage 722. However, in other examples, the central body 318 may not include the proximal passage 722 and the securing member 724 may comprise integral geometry of the central body 318.

In some examples, the securing member 724 may facilitate a secure bond between the outer sheath 312 and a first sleeve element 726 (FIG. 25). For example, the securing member 724 may contact and engage an outer surface or circumference of the first sleeve element 726, and an inner surface or circumference of the outer sheath 312 may be bonded to the outer surface or circumference of the first sleeve element 726. In one specific example, the outer sheath 312 may be bonded to the first sleeve element 726 via the application of heat, such as in an example where the outer sheath 312 comprises a type of heat shrinkable tubing such as fluorinated ethylene propylene (“FEP”), among other heat shrinkable materials. In another example, the outer sheath 312 may reflowed onto the first sleeve element 726 via the application of heat, such as in an example where the outer sheath 312 comprises a thermoplastic material such as, but not limited to, polyether block amides (e.g., “PEBAX”) or high-density polyethylene.

In some examples, the first sleeve element 726 may be a hypo tube comprised of metallic materials including, but not limited to, stainless steel, titanium, or coated or non-coated steel. In any example including the first sleeve element 726, the inner surface or circumference may be sized and shaped to provide clearance for the outer sheath 312 to translate freely therein.

In some examples, the securing member 724 may engage the first sleeve element 726 via one or more set screws 728 (FIG. 25) passing through a sidewall of the securing member 724. In other examples, the securing member 724 may engage the first sleeve element 726 via a press or friction fit, or through laser welding, reflow, or adhesive bonding, among others surface bonding techniques. In some examples, a proximal terminus or end of the first sleeve element 726 may be located within a distal end portion 352 of the outer housing 308 and a distal terminus of the first sleeve element 726 may be located within the second component 305 of the scope adapter 301 to help brace or otherwise strengthen the inner sheath 114 and the distal supply line 361 in transition regions or zones between the central body 318 and the outer housing 308, and the central body 318 and the scope adapter 301.

Further, in some such examples, an outer surface or circumference of the outer sheath 312 may be further be bonded directly to the central body 318 in a location or position distal to the securing member 724 and the proximal passage 722. In some other examples, the securing member 724 may directly contact and engage an outer surface or circumference of the outer sheath 312 directly, which may eliminate the first sleeve element 726. In further additional examples, the central body 318 may not include the proximal passage 722, the securing member 724, or the first sleeve element 726, and an outer circumference or surface of the outer sheath 312 may simply be bonded directly to the central body 318.

In some examples, the sampling system 306 may further include a second sleeve element 313 (FIG. 24) that is bonded, such as via laser welding, reflow, or adhesive bonding, among other surface bonding techniques, within the second component 305 of the scope adapter 301. In such examples, the second sleeve element 313 may help to increase the rigidity and axial deflection resistance of the probe assembly 310 near the scope adapter 301 and thereby reduce the chance of kinking and/or breakage of the outer sheath 112 and/or the inner sheath 114 contained therein. The second sleeve element 313 may be made from various materials including, but not limited to, stainless steel, nitinol, or titanium, among others.

Turning now to the inner sheath 314, the inner sheath 314 may extend from its proximal terminus within the outer housing 308, such as shown in FIG. 23, pass longitudinally and distally through both the scope adapter 301 and the central body 318, such as shown between FIGS. 24 and 25, and terminate at the distal tip 116, such as shown in FIGS. 26 and 28. Like the inner sheath 114, a proximal terminus of the inner sheath 314 may be bonded or secured, such as via laser welding, adhesives, or reflowing, among other surface bonding techniques, within a first recess 369 (FIG. 23) of a fluid connector 370 (FIG. 23); and, like the fluid connector 170, the fluid connector 370 may fluidly connect, via an exhaust passage 371 (FIG. 23), an exhaust lumen 710 defined by a size differential between an inner diameter of the inner sheath 314 and an outer diameter of the distal supply line 261 (FIGS. 23, 26 and 28) to an exhaust line 341 (FIG. 23) bonded or secured, such as via laser welding, adhesives or reflowing, among other surface bonding techniques, within a second recess 373 (FIG. 23) of the fluid connector 370.

Finally, the fluid connector 370, like the fluid connector 170, may also be potted with an adhesive or other sealing material at the location where the distal supply line 361 enters the first recess 369 to prevent leakage of exhaust gases into the outer housing 308. The exhaust line 341 may generally be similar, or identical, to the exhaust line 141 previously discussed above, and the tube 307 may be potted with an adhesive or sealing material at the location where the exhaust line 141 enters the tube 307 to prevent leakage of the exhaust gases which, as previously noted, travel proximally through the tube 307 to the refrigerant system 104 for venting to the atmosphere.

However, unlike the fluid connector 170 contained within the position controller 118 of the sampling system 106, the fluid connector 370 of the sampling system 306 is immovable with respect to the outer housing 108. For example, the fluid connector 170 may define a protrusion 761 (FIG. 23) sized and shaped to be engaged and retained within an inner housing 763 (FIG. 23) defined by geometry of the outer housing 308. Thus, in view of the above, the outer housing 308 may be translated proximally and distally in a direction parallel to the longitudinal axis A1 to, in turn, cause the inner sheath 314 and the distal tip 316 to extend or retract within the outer sheath 312.

In further examples, the sampling system 306 may also include a third sleeve element 740 (FIGS. 23 and 25). The third sleeve element 740 may encompass a longitudinal length of the inner sheath 314 generally between a proximal end of the central body 318 and the fluid connector 370, such as to help increase the axial elongation or deflection resistance of the inner sheath 314 between the fluid connector 370 and the central body 318. In some examples, a proximal terminus or end of the third sleeve element 740 may be bonded, such as via adhesives or reflowing, among other surface bonding techniques, within a third recess 742 (FIG. 23) of the fluid connector 370, and a distal terminus of the third sleeve element 740 may be bonded within the central body 318 near its proximal end, such as illustrated in FIG. 24. Additionally, in such examples, an inner surface of the third sleeve element 740 may be bonded to an outer surface of the inner sheath 314 so that the third sleeve element 740 translates with the inner sheath 314 during movement of the outer housing 308 with respect to the central body 318.

In still further examples, the sampling system 306 may include a fourth sleeve element 745 (FIG. 23) to help strengthen the connection or bond between the inner sheath 314 and the fluid connector 370 and/or the third sleeve element 740. For example, as shown in FIG. 23, a proximal terminus of the fourth sleeve element 745 may be located between, and bonded to, both the first recess 769 of the fluid connector 370 and the inner sheath 314 to sandwich the inner sheath 314 therebetween, and a distal terminus of the fourth sleeve element 745 may be bonded to both the inner sheath 314 and/or the third sleeve element 740. Such a configuration may also help to increase the axial stiffness and/or deflection resistance of the inner sheath 314 in a region or zone near the fluid connector 370.

As previously noted above, the outer housing 308 may translate proximally and distally around the central body 318. For example, the outer housing 308 may define guide passage 746 (FIG. 24) extending within the distal end portion 352 of the outer housing 308 sized and shaped to allow free translation of the central body 318 therein. In some examples, the proximal portion of the central body 318 and the guide passage 746 may define various complimentary cross-sectional shapes including, but not limited, to squares, rectangles, circles, ellipsoids, or triangles, among others. In some examples, the central body 318 may further include one or more guide elements 747 (FIG. 24), and/or the outer housing 308 may further include one or more guide projections 748 (FIG. 24) that may slide along the outer housing 308 within the guide passage 746 to help stabilize the central body 318.

In some examples, the central body 318 may include a plurality of indicators 760 (FIG. 24). Each of the plurality of indicators 760 may be positioned to provide a clear visual indication of an axial position of the distal tip 316 with respect to the outer sheath 312, such as when one of the plurality of indicators 760 is aligned with a known reference point on or near the outer housing 308. In some examples, the known reference point may be a window defined through the outer housing 308, such as to enable a user to view one of the plurality of indicators within the guide passage 746. In another example, the known reference point may generally be a location distally adjacent to the distal end portion 352 of the outer housing 308. In further examples, the known reference point may be a window 756 of a pawl assembly 750.

In some examples, the plurality of indicators 760 may include one, two, three, four, five, six or other numbers of visual markings each spaced equidistantly, or non-equidistantly, apart in a direction parallel to the longitudinal axis A1. In some examples, each of the plurality of indicators 760 may be located within an inclusive range of about 0.5 centimeters to about 4 centimeters apart from one along the central body 318. In one specific example, the plurality of markings 150 may each be spaced about 1 centimeter apart.

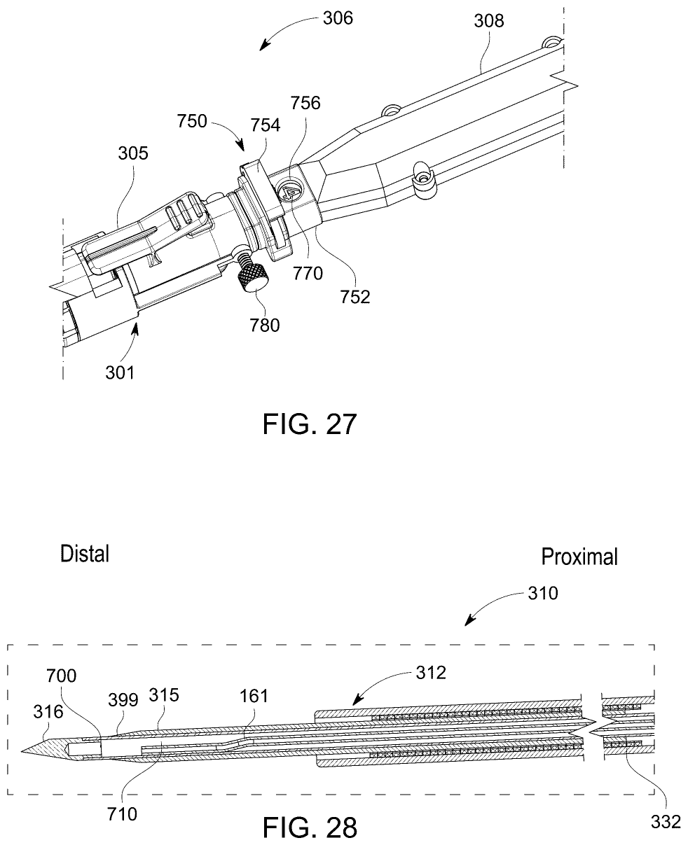

In one example, such as shown in FIGS. 26, the plurality of indicators 760 may comprise a first marking 762, a second marking 764, a third marking 766, a fourth marking 768, and a fifth marking 770 (FIGS. 24 and 27), respectively. In some such examples, the first marking 762 may be labeled on the central body 318 as “0”, the second marking 764 may be labeled on the central body 318 as “1”, the third marking 766 may be labeled on the central body 318 as “2”, the fourth marking 768 may be labeled on the central body 318 as “3”, and the fifth marking 770 may be labeled on the central body 318 as “4” t thereby numerically indicate the longitudinal distance, such as measured in centimeters, by which the distal tip 316 protrudes from the outer sheath 312.

By way of further example, the distal tip 316 may be in a fully retracted position, as shown in FIG. 26, when the outer housing 308 is in the first position (e.g., when the first marking 762 labeled as “0” is aligned with the known reference point), and the distal tip 116 may be a fully extended position, as shown in FIG. 28, when the outer housing 308 is in the fifth position (e.g., when the fifth marking 770 labeled as “4” is aligned with the known reference point). In one specific example, the distal tip 316 may extend about 4 centimeters distally beyond the outer sheath 312 when the outer housing 308 is in its fifth position. In still further examples, the distal tip 116 may be positioned in several different partially extended positions, such as when the second marking 764, the third marking 766, or the fourth marking 768 are aligned with the known reference point (e.g., when the outer housing 308 is in a second position, a third position, or a fourth position).

In some examples, the sampling system 306 may further prevent over extension of the distal tip 316. First, for example, the central body 318 may define a plurality of teeth 376 spaced equidistantly apart. Second, the sampling system 306 may include a pawl assembly 750 that may selectively engage the plurality of teeth 758 to adjustably limit distal translation of the outer housing 308 along the central body 318. The pawl assembly 750 may include a pawl housing 752 and a pawl element 754. The pawl housing 752 may be structure encompassing an outer perimeter of a portion of the central body 318. The pawl housing 752 may be sized and shaped to be freely translatable proximally and distally about the central body 318.

The pawl element 754 may be a locking device movably connected to the pawl housing 752 to disengage or engage the plurality of teeth 758 of the central body 318. In some examples, the pawl element 754 may be a push-button structure that is translatable in direction orthogonal to the longitudinal axis A1 (FIGS. 23 and 24) to cause the pawl element 754 to disengage the plurality of teeth 758. In such an example, the pawl element 754 may be spring loaded, or otherwise biased, such that the pawl element 754 is normally in contact with the plurality of teeth 758, such as until the pawl element 754 is manually depressed by a user. In some examples, the pawl housing 752 may further define a window 756 (FIGS. 24 and 27) so that a user may visualize one of the plurality of indicators 270 therein.

In view of the above, the pawl assembly 750 may help to increase patient safety by enabling a user to selectively control the maximum penetration depth of the distal tip 116 (e.g., the longitudinal distance by which the distal tip 116 protrudes from the outer sheath 112). For example, a user may first depress the pawl element 754 into the pawl housing 752 to cause the pawl element 754 to disengage the plurality of teeth 758. Then, with the pawl element 754 depressed, the user may freely translate the pawl housing 752 proximally or distally along the central body 318 until the window 356 displays a desired sampling or ablation position for the distal tip 316. Next, the user may release the pawl element 754 to cause the pawl element 754 to reengage the plurality of teeth 758 and prevent relative movement between the pawl assembly 750 and the central body 318. The user may then translate the outer housing 308 distally until the distal end portion 352 contacts the pawl assembly 350, thereby eliminating the possibility of overextending the distal tip 316.

However, in other examples, the outer housing 308 may be selectively retained in, or otherwise limited to, various positions with respect to the central body 318 using a variety of other mechanical techniques or devices known in the art, such as, among others, a ratcheting engagement between the outer housing 308 and the central body 318, a pin-and-hole detent mechanism between the outer housing 308 and the central body 318, or a magnetic latching and/or braking mechanism between the outer housing 308 and the central body 318.

In some examples, as previously mentioned above, the sampling system 306 may include an adjustment mechanism 321. The adjustment mechanism 321 may be configured to enable a user to selectively position the outer sheath 312 relative to a distal end of the endoscope 302, specifically when the sampling system 306 is secured to the endoscope 302. First, in such examples, the second component 305 of the scope adapter 301 may define a proximal opening 782 (FIG. 24) that may be sized and shaped to translatably receive a distal portion 784 (FIGS. 24 and 30) of the central body 318. In some examples, the distal portion 784 of the central body 318 and the proximal opening 782 may define various complimentary cross-sectional shapes, such as including, but not limited to, squares, rectangles, circles, ellipsoids, or triangles, among others.

Second, the adjustment mechanism 321 may include an engagement member 780. The engagement member 780 may be component or structure that is movable into the proximal opening 782 to engage, or disengage, the distal portion 784 of the central body 318 to prevent relative movement, or enable relative movement, respectively, between the central body 318 and the scope adapter 301. In some examples, such as shown in FIGS. 24, 27, and 31), the engagement member 780 may comprise one or more threaded fasteners that may rotatably engage a plurality of threads defined through a sidewall of the second component 305. Further, in some examples, the central body 318 may also define a projection 786 (FIGS. 24 and 30-31) that may be sized and shaped to contact the second component 305 of the scope adapter 301 to limit distal translation of the central body 318 with respect the scope adapter 301.

However, in other examples, the central body 318 may be selectively retained in, or adjusted between, various longitudinal or axial positions with respect to the scope adapter 301 using other mechanical techniques or devices known in the art, such as, but not limited to, a ratcheting interface between the central body 318 and the scope adapter 301, a pin-and-hole detent mechanism between the central body 318 and the scope adapter 301, or a magnetic latching and/or braking mechanism between the central body 318 and the scope adapter 301, among others.

In view of the above, the adjustment mechanism 321 may enable a user to change a longitudinal position of the outer sheath 312 with respect to the working channel of the endoscope 302, or other surgical scopes and/or instruments. More specifically, the adjustment mechanism 321 may provide a user with the ability to precisely adjust, after the sampling system 306 is secured to the endoscope 302 with the scope adapter 301, a specific longitudinal distance by which the outer sheath 312 protrudes beyond the endoscope 302. Moreover, the adjustment mechanism 321 may allow a user to, for example, detach the first component 303 from the second component 305 multiple times during a procedure without changing a pre-set relative position of outer sheath 312. In other words, each time the probe assembly 310 is reinserted into the endoscope 302 through the first component 303, the distal end of the outer sheath 312 will automatically return to the same position within a patient.

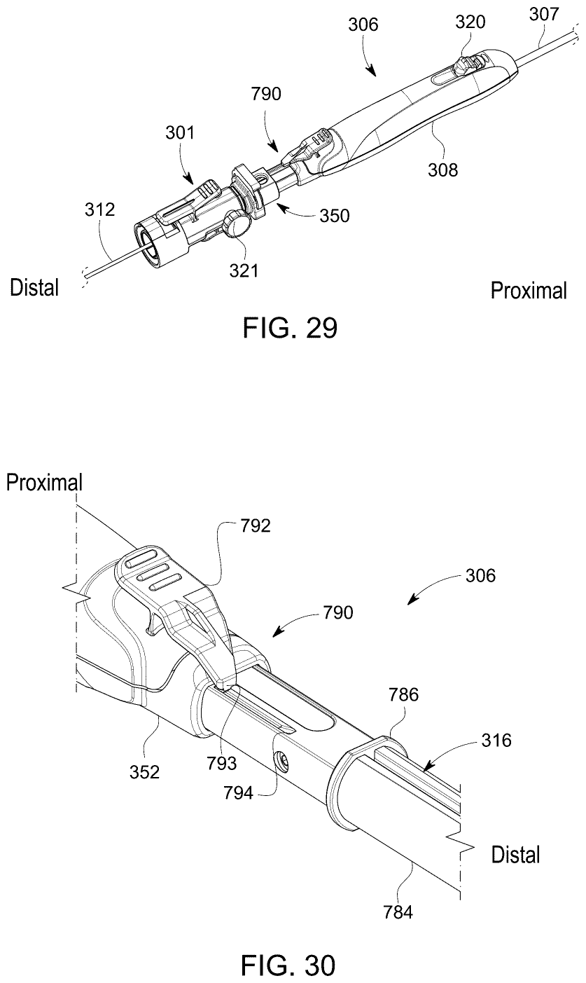

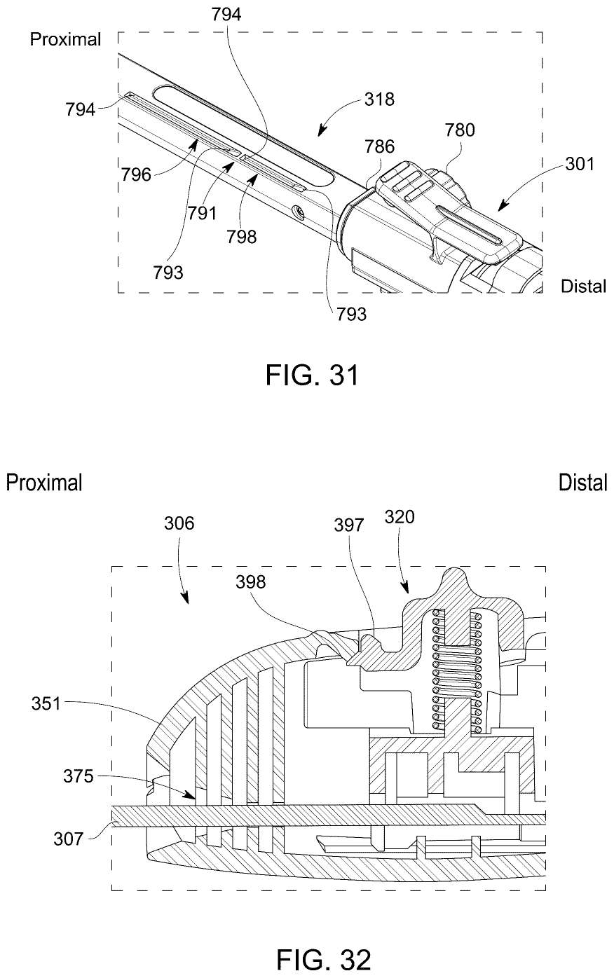

Finally, in some examples, the sampling system 306 may include a travel limiting mechanism 790 (FIGS. 29-31). In contrast to the pawl assembly 350, which, as noted above, may prevent over-extension of the distal tip 116 from the outer sheath 312, the travel limiting mechanism 790 may prevent over-retraction of the distal tip 316 back into the outer sheath 312. The travel limiting mechanism 790 may include a plurality of grooves 791 (FIG. 31) defined by the central body 318, and an engagement device 792 (FIGS. 30-31) disposed on the distal end portion 352 of the outer housing 308.

The engagement device 792 may be a pivotable structure, such as a lever, including a projection 793 that may be sized and shaped to translate within and axially through each of the plurality of grooves 791 during movement of the outer housing 308 relative to the central body 318. The engagement device 792 may be configured to bias the projection 793 downwardly toward the central body 318 so that the projection 793 is normally in contact with the central body 318. In other words, unless the engagement device 792 is pivoted, depressed, or otherwise manipulated by a user to move the projection 793 away from the central body 318, the projection 793 may be in contact with the central body 318 when the central body 318 is at least partially received within the outer housing 308.

The plurality of grooves 791 may comprise a series of axially aligned, and also axially spaced, elongated tracks or depressions which extend within the central body 318 in a direction parallel to the longitudinal axis A1. In some examples, the plurality of grooves 791 may include one, two, three, four, five, six or other numbers of individual grooves spaced equidistantly or non-equidistantly apart from one another. Additionally, each of the plurality of grooves 791 may include a distal angled surface 794 (FIG. 31) and a proximal vertical surface 795 (FIG. 31). The distal angled surface 794 may be configured to enable the projection 793 of the engagement device 792 to freely slide between each of the plurality of grooves 791 when the outer housing 308 moves in a distal direction (e.g., toward the central body 318).

However, the proximal vertical surface 795 of each of the plurality of grooves 791 may be configured to stop the projection 793 of the engagement device 792 from moving between any of the plurality of grooves 791 when the outer housing 308 moves in a proximal direction (e.g., away from the central body 318). Accordingly, when a user translates the outer housing 308 distally to extend the distal tip 316 into the tissue, the engagement device 392 may proceed unimpeded, but when the outer housing 308 beings to move proximally, such as after the refrigerant controller 320 has been utilized to adhere a tissue sample to the distal tip 316, the projection 793 will soon contact the proximal vertical surface 795 of one of the plurality of grooves 791, thereby stopping the outer housing 308 from being translated further until a user operates the engagement device 392 to cause the projection 793 to disengage (e.g., lift up and over) the proximal vertical surface 795.

In view of the above, it also logically follows that, in some examples, the plurality of grooves 791 may be configured to correspond to various pre-defined positions of the probe assembly 310 and/or the outer housing 308. For example, if the plurality of indicators 760 on the central body 318 includes one indicator for visually indicating to a user that the distal tip 116 is in a fully extended position, the plurality of grooves 791 may include only one single groove, with the proximal vertical surface 795 thereof being axially positioned to contact the projection 793 when the outer housing 308 reaches a fully extended position along the central body 318.

As another example, if the plurality of indicators 760 of the central body 318 includes two indicators for visually indicating to a user when the distal tip 116 is in a fully extended position and a partially extended position, respectively, the plurality of grooves 791 may include a first groove 796 and a second groove 798, respectively, such as shown in FIG. 31, with the proximal vertical surface 795 of the first groove 796 being axially positioned to contact the projection 793 when the outer housing 308 reaches a partially extended position and proximal vertical surface 795 of the second groove 798 being axially positioned to contact the projection 793 when the outer housing 308 reaches a fully extended position.

In view of the above, the travel limiting mechanism 790 may be highly advantageous in examples where the outer sheath 312 and the inner sheath 314 and/or the thermal jacket 315 are not configured to provide a sufficient air gap therebetween to accommodate a tissue sample adhered to, or otherwise collect by, the distal tip 316. For example, by preventing the outer housing 308 from retracting the distal tip 316 back into the outer sheath 312, the travel limiting mechanism 790 may prevent the tissue sample from being crushed between the outer sheath 112 and the inner sheath 314 and/or being dislodged from the distal tip 316. Moreover, by preventing the distal tip 316 from being retracted back into the outer sheath 312, a user may pull proximally on the outer housing 308 with significant force without causing the distal tip 316 to retract, which may be helpful in procedures involved relatively tough tissue that may require a degree of pulling force to remove.

In an example method of performing a surgical procedure with the cryobiopsy device 300, a user may first open a sterile package containing a sterile portion which houses the sampling system 306, the refrigerant system 104, and/or the scope adapter 301. In some examples, the refrigerant cartridge 126 and the cartridge nest 122 may be supplied within a non-sterile portion of the sterile package. Next, the user may insert the cartridge nest 122, with the refrigerant cartridge 126 contained therein, into the base housing 124 to establish fluid communication between the refrigerant system 104 and the sampling system 306. The user may then guide the endoscope 302 and the probe assembly 310 to a target anatomical location, such as by first inserting the endoscope 302 into a patient through a natural body orifice, and subsequently inserting the probe assembly 310 through the probe opening 309 of the first component 303 before securing the second component 305 to the first component 303, thereby coupling the sampling system 306 to the endoscope 302.

In some examples, the user may then advance the outer sheath 312 of the probe assembly 310 distally beyond a distal end of the endoscope 302, such as by utilizing the adjustment mechanism 321, to a location adjacent the tissue to be biopsied, ablated, or otherwise treated. Next, the user may translate the outer housing 308 toward the scope adapter 301 to cause the distal tip 316 to emerge from the outer sheath 112 and into contact with tissue. In some examples, a user may first utilize the pawl assembly 350 to prevent inadvertent over-extension of the distal tip 316 into the target tissue. The user may then translate and/or depress the refrigerant controller 320 to open a valve assembly within the sampling system 306, thus allowing refrigerant from the refrigerant system 104 to begin rapidly cooling the distal tip 316, and/or or begin dispersing gas directly into tissue, to ablate or adhere tissue to the distal tip 316. In some examples, the refrigerant controller 320 may be locked into an open position without the continuous application of pressure thereon. In some examples, the refrigerant controller 320 may be left in an open position within an inclusive range of about 2 seconds to about 2 minutes to adequately treat and/or bond tissue to the distal tip 316.

The probe assembly 310 may then be withdrawn through the working channel of the endoscope 302, such as by first disconnecting the first component 303 from the second component 305. Alternatively, in some examples where the probe assembly 310 may be configured to have a sufficient air gap between the outer sheath 312 and the inner sheath 314, a tissue sample adhered to the distal tip 316 may be removed by advancing the outer sheath 312 distally over the distal tip 316, such by utilizing the adjustment mechanism 321, or by proximally retracting the distal tip 316 back into the outer sheath 312 or pulling the outer housing 308 in a proximal direction. Finally, the user may scrape or otherwise remove the tissue sample from distal tip 316.

With regard the above, the inventors have also appreciated that a similar method may be performed to ablate tissue to address a wide variety of medical conditions, such as, but not limited to, those disclosed further below. For example, if the distal tip 316 is instead configured to be completely hollow, or may otherwise define a passage or bore extending through distal end surface of the distal tip 316, refrigerant may flow distally therebeyond and into contact with tissue when the refrigerant controller 320 is in a fully open or partially open position.

Still further, inventors have appreciated that the sterile package in which the cryobiopsy device 100 or the cryobiopsy device 300 is supplied may, in some examples, also include a disposable tissue removal device, such as a tissue scraper for aiding the user in separating tissue from the distal tip thereof. In some examples, such a tissue removal tool may be a customized tool adapted to help reduce the shear-forces imparted to a collected tissue sample to thereby reduce the chance of imparting crush artifacts to the collected tissue during removal. In one such example, a tweezers with pivotable jaws sized and shaped to close around and circumferentially engage, and slide along, the inner sheath, outer sheath, or distal tip may be provided. In another example, a sample collection vial may be provided which includes a tissue removal mechanism, such as an opening or aperture including a flange or other projection or protrusion adapted to engage a collected tissue sample to cause it to fall into the vial. In some examples, such as vial may also be filled with a preservation agent such as formalin.

FIG. 33 is a cross-section of a second axial tube valve assembly 400 in an open position and FIG. 34 is a cross-section of a second axial tube valve assembly 400 in a closed position. FIGS. 33-34 are discussed below concurrently. In some examples, the first axial tube valve assembly 154 (or the first axial tube valve assembly 354) may be replaced by the second axial tube valve assembly 400. The first axial tube valve assembly 354 may generally be similar, or identical, to first axial tube valve assembly 154, at least in that the second axial tube valve assembly 400 relies on a tubular element 412 which translates proximally and distally, as indicated by arrow 413, to enable or disable a flow 416 of compressed gas to a distal supply line of a probe assembly, such as, but not limited to, the distal supply line 161 or the distal supply line 361.

The second axial tube valve assembly 400 may include a filter 404, a first seal 406, a second seal 408, and a spacer 410 each received within the valve body 402. The valve body 402 may also define a first passage 418 for guiding the tubular element 412, and the spacer 410 may define a second passage 411 in fluid communication with a proximal supply line 414 connected to the valve body 402. The first seal 406 and the second seal 408 are positioned on opposite sides of the spacer 410 within the valve body 402 to ensure gas may escape the valve body 402 only by passing through the tubular element 412.

As such, when an aperture 407 in a sidewall of the tubular element 412 is aligned with the second passage 411, the second axial tube valve assembly 400 is open as gas flows through the filter 404, and when the aperture 407 is spaced proximally away from the second passage 411 beyond the second seal 408, the second axial tube valve assembly 400 is closed. The valve body 402 may also include the end cap 162 (FIG. 6) to ensure that the second seal 408 is securely retained within the valve body 403.

As may by appreciated from FIGS. 33-34, and in contrast to the first axial tube valve assembly 154 and the first axial tube valve assembly 354, the valve body 402 may be made from a single, unitary piece of material rather than being comprised of two separate components, such as the first body portion 158 (FIG. 6) and the second body portion 159 (FIG. 6). Finally, various components of the second axial tube valve assembly 400 may be identical to various components of the first axial tube valve assembly 154 sharing the same name, such as, but not limited to, the filter 404, the first seal 406, the second seal 408, and/or the tubular element 412.

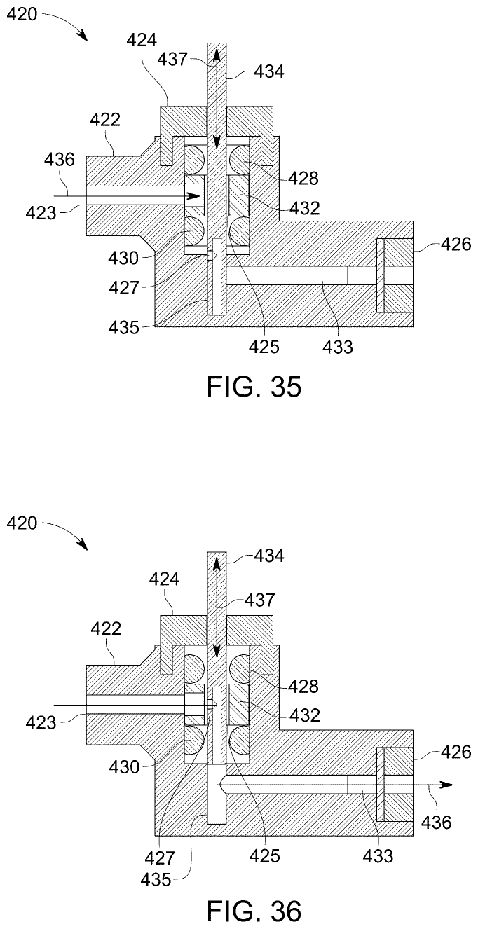

FIG. 35 illustrates a first vertical tube valve assembly 420 in a closed position, and FIG. 36 illustrates the first vertical tube valve assembly 420 in an open position. FIGS. 35-36 are discussed below concurrently. In some examples, the first axial tube valve assembly 154 (or the first axial tube valve assembly 354) may be replaced by the first vertical tube valve assembly 420. The first vertical tube valve assembly 420 includes a tubular element 434 which, unlike the first axial tube valve assembly 154, translates orthogonally to the longitudinal axis A1 of the sampling system 106 (or the sampling system 306), as indicated by arrow 437, to enable or disable a flow 436 of compressed gas to a distal supply line of a probe assembly, such as, but not limited to, the distal supply line 161 or the distal supply line 361 previously described above.

The first vertical tube valve assembly 420 may include a filter 426, a first seal 428, a second seal 430, and a spacer 432 each received within a valve body 422. The valve body 422 may include a first passage 435 for guiding the tubular element 434, the spacer 410 may define a second passage 425 in fluid communication with a proximal supply passage 423 defined by the valve body 402, and the valve body 422 may further include a third passage 433 for guiding the flow 436 of gas to the filter 426. The first seal 428 and the second seal 430 are positioned on opposite sides of the spacer 432 within the valve body 402 to ensure gas may escape the valve body 402 only by passing through the tubular element 434.

As such, when an aperture 427 in a sidewall of the tubular element 434 is aligned with the third passage 433, the first vertical tube valve assembly 420 is open, and when the aperture 427 is spaced away from the second passage 411 and above the second seal 430, the first vertical tube valve assembly 420 is closed. The valve body 422 may also include an end cap 424 to ensure that the first seal 428 is securely retained within the valve body 422. Finally, various components of the first vertical tube valve assembly 420 may be identical to various components of the first axial tube valve assembly 154 sharing the same name, such as, but not limited to, the filter 426, the first seal 428, the second seal 430, and/or the tubular element 434.

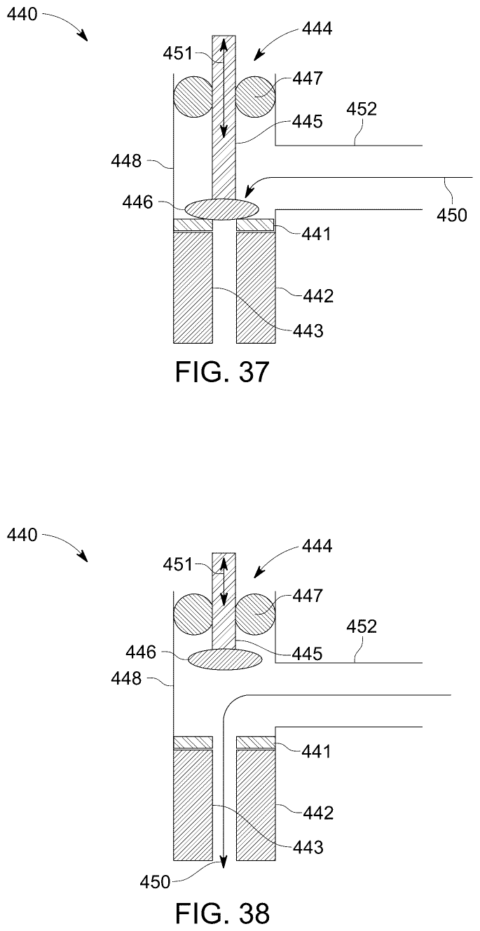

FIG. 37 illustrates a first plunger valve assembly 440 in a closed position and FIG. 38 illustrates the first plunger valve assembly 440 in an open position. FIGS. 37-38 are discussed below concurrently. In some examples, the first axial tube valve assembly 154 (or the first axial tube valve assembly 354) may be replaced by the first plunger valve assembly 440. The first plunger valve assembly 440 includes a plunger shaft 445 and a plunger head 446 which translates orthogonally to the longitudinal axis A1 (FIGS. 7-8) of the sampling system 106, as indicated by arrow 451, to enable or disable a flow 450 of gas to a distal supply line of a probe assembly, such as, but not limited to, the distal supply line 161 previously described above.

The plunger head 446 may form different shapes, such as an ellipsoidal shape, a circular or spherical shape, a semi-spherical or a semi-hemispherical shape, or other oblong or elongated non-polygonal shapes. The first plunger valve assembly 440 further includes a valve body 448 with a spacer 442 and a seal 447 received therein. In some examples, the spacer 442 includes a deformable surface 441 positioned to be engaged by the plunger head 446 to create an air-tight seal therebetween. However, in other examples, the plunger head 446 may alternatively (e.g., instead of the spacer 442) include the deformable surface 441 to create an air-tight seal between the spacer 442 and the plunger head 446.

The deformable surface 441 may be comprised of a compliant gasket material capable of creating an air-tight seal, such as, but not limited to, polymeric materials such as, but not limited to rubber or elastomers (e.g., Ethylene-Propylene Rubber (“EPM”), Ethylene Propylene Diene Monomer (“EPDM”), silicone, nitrile, polychloroprene, etc.), among others.

The spacer 442 defines a first passage 443 guiding the flow 450 to a downstream filter and a distal supply line, such as filter 160 and the distal supply line 161. The valve body 448 may also define, or be connected to, a second passage 452 functioning as a proximal supply line, such as the proximal supply line 140. The seal 447 is positioned about the plunger shaft 445 and engages the valve body 448 to ensure gas may only escape the valve body 448 through the first passage 443. As such, when the plunger head 446 is in contact with the deformable surface 441, the first plunger valve assembly 440 is closed, and when the plunger head 446 is spaced away from the deformable surface 441, the first plunger valve assembly 440 is open.

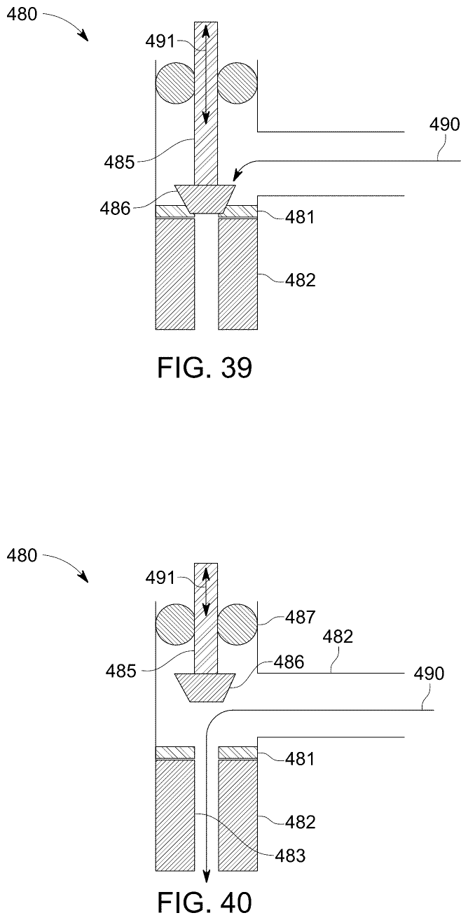

FIG. 39 illustrates a second plunger valve assembly 480 in a closed position, and FIG. 40 illustrates the second plunger valve assembly 480 in an open position. FIGS. 39-40 are discussed below concurrently. In some examples, the first axial tube valve assembly 154 (or the first axial tube valve assembly 354) may be replaced by the second plunger valve assembly 480. The second plunger valve assembly 480 includes a plunger shaft 485 and a plunger head 486 which translates orthogonally to the longitudinal axis A1 (FIGS. 8-9) of the sampling system 106, as indicated by arrow 491, to enable or disable a flow 490 of gas to a distal supply line of a probe assembly, such as, but not limited to, the distal supply line 161 previously described above.

In some examples, the second plunger valve assembly 480 may be similar to the first plunger valve assembly 440 discussed above, except in that the plunger head 486 may form a trapezoidal shape, a rectangular or cuboidal shape, a triangular shape, or other polygonal shapes. As such, when the plunger head 486 is in contact with a deformable surface 481 of a spacer 482, the second plunger valve assembly 480 is closed, and when the plunger head 486 is spaced away from the deformable surface 481, the second plunger valve assembly 480 is open. However, in other examples, the plunger head 486 may alternatively (e.g., instead of the spacer 482) include the deformable surface 481 to create an air-tight seal between the spacer 482 and the plunger head 486.

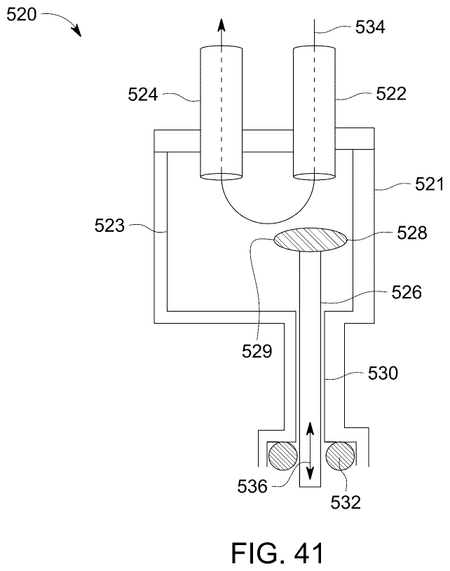

FIG. 41 is a cross-section of a third plunger valve assembly 520 in an open position, according to one example of the present disclosure. In some examples, the first axial tube valve assembly 154 (or the first axial tube valve assembly 354) may be replaced by the third plunger valve assembly 520. The third plunger valve assembly 520 includes a plunger shaft 526 and a plunger head 528 including a deformable outer surface 529, which translates orthogonally to the longitudinal axis A1 of the sampling system 106, as indicated by arrow 536, to enable or disable a flow 534 to a distal supply line of a probe assembly, such as, but not limited to, the distal supply line 161 previously described above.

In some examples, the plunger head 528 may include a deformable outer surface 529. The deformable outer surface 529 may be comprised of a compliant gasket material capable of creating an air-tight seal, such as, but not limited to, polymeric materials such as, but not limited to, rubber or elastomers (e.g., ethylene-propylene rubber (“EPM”), ethylene propylene diene monomer (“EPDM”), silicone, nitrile, neoprene, etc.), among others. The third plunger valve assembly 520 also includes a valve body 521 defining a chamber 523 and a first passage 530 for guiding translation of the plunger shaft 526 within the valve body.

The third plunger valve assembly 520 further includes a second passage 522 functioning, or connected to, a proximal supply line, such as the proximal supply line 140, and a third passage 524 functioning, or connected to, a filter and distal supply line, such as, but not limited to, the filter 160 and the distal supply line 161. The seal 532 is positioned about the plunger shaft 526 and engages the valve body 521 to ensure gas may only escape the valve body 521 through the third passage 524. As such, when the deformable outer surface 529 of the plunger head 528 is in contact with the second passage 522, the third plunger valve assembly 520 is closed, and when the plunger head 528 is spaced away from the second passage 522, the third plunger valve assembly 520 is open.

Alternatively, in other examples, the second passage 522 (e.g., instead of the plunger head 528) may include the deformable outer surface 529. In such examples, when the second passage 522 is in contact with the plunger head 528, the third plunger valve assembly 520 is closed, and when the plunger head 528 is spaced away from the second passage 522, the third plunger valve assembly 520 is open.

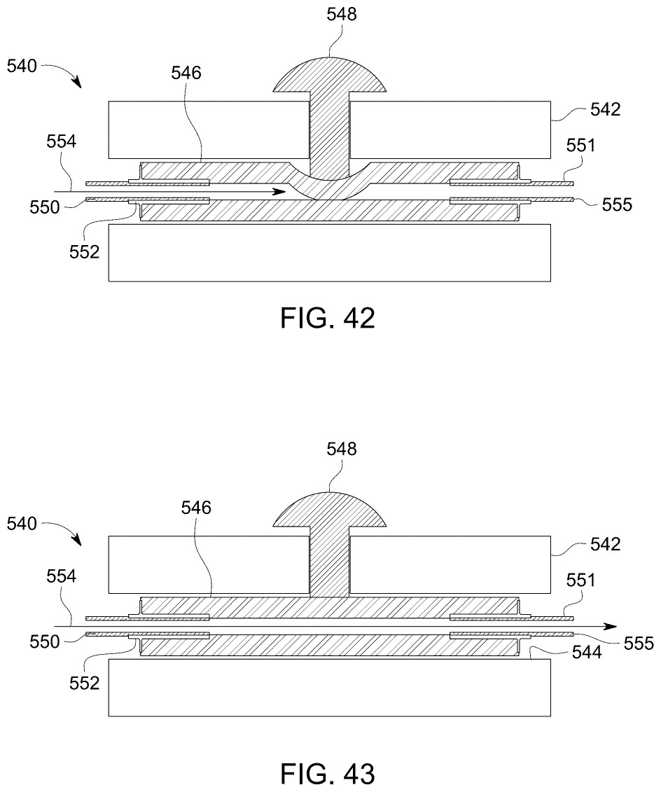

FIG. 42 is a cross-section of a first crush valve assembly 540 in a closed position, and FIG. 43 is a cross-section of the first crush valve assembly 540 in an open position. FIGS. 42-43 are discussed below concurrently. In some examples, the first axial tube valve assembly 154 (or the first axial tube valve assembly 354) may be replaced by the first crush valve assembly 540. The first crush valve assembly 540 includes a refrigerant controller 548 and a valve body 542 housing a deformable tube 546 which deforms to enable or disable a flow 554 of gas to a probe assembly, such as the probe assembly 110 or 310.

The deformable tube 546 may be made from any soft, deformable tubing material designed to collapse or compress under pressure, such as, but not limited to, rubber or elastomers (e.g., ethylene-propylene rubber (“EPM”), ethylene propylene diene monomer (“EPDM”), silicone, nitrile, neoprene, etc.). The refrigerant controller 548 may be any push button structure that may impart a crush force to the deformable tube 546 in a direction perpendicular to the longitudinal axis A1 of the sampling system 106 (or the sampling system 306). As the deformable tube 546 is under significant pressure from compressed gas, the refrigerant controller 548 may include various mechanisms that may limit or prevent return travel when engaging the deformable tube 546, such as detents, a “push-push” or “click-on/click-off” style mechanism, a depressible slide-style mechanism as described with to the refrigerant controller 120 of the sampling system 306, among others.

The deformable tube 546 may also be coupled to a proximal supply line 550 for supplying pressurized gas, such as the proximal supply line 140, and a distal supply line 551 for sending pressurized gas to a filter and distal supply line, such as the filter 160 and the distal supply line 161. The proximal supply line 550 may be secured to the deformable tube 546 via a first coupling member 552 and the distal supply line 551 may be secured to the deformable tube 546 via a second coupling member 555. In various examples, the first coupling member 552 and the second coupling member 555 may be representative of adhesives, polymeric or elastomeric connectors, or metallic connectors, among others. In view of the above, when the refrigerant controller 548 is in contact with the deformable tube 546, the first crush valve assembly 540 is closed, and the refrigerant controller 548 is spaced away from the deformable tube 546, the first crush valve assembly 540 is open.

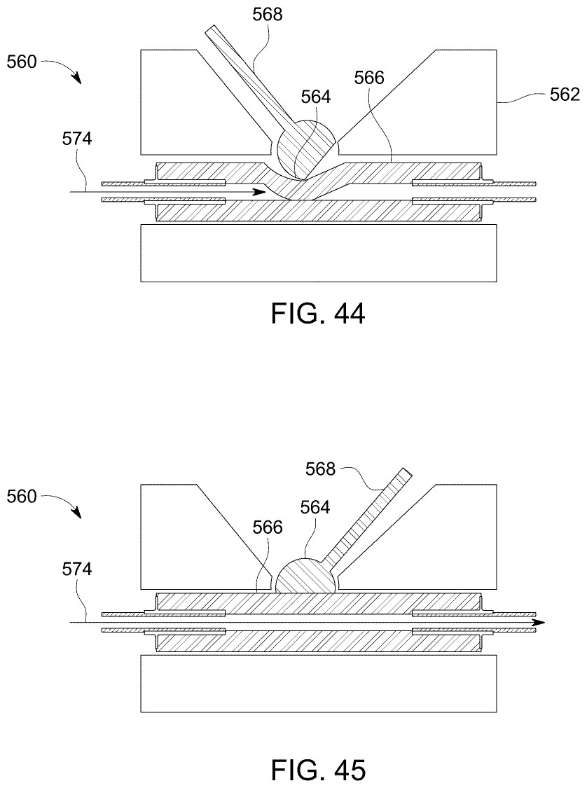

FIG. 44 is a cross-section of a second crush valve assembly 560 in a closed position, and FIG. 45 is a cross-section of the second crush valve assembly 560 in an open position. FIGS. 44-45 are discussed below concurrently. In some examples, the first axial tube valve assembly 154 (or the first axial tube valve assembly 354) may be replaced by the second crush valve assembly 560. The second crush valve assembly 560 includes a refrigerant controller 568 and a valve body 562 housing a deformable tube 566 which deforms to enable or disable a flow 574 of gas to a probe assembly, such as the probe assembly 110 or 310.

The second crush valve assembly 560 may be similar to the first crush valve assembly 540 previously described above, except in that the refrigerant controller 568 may pivot or rotate to cause a cam 564 to impart a crush force to the deformable tube 566 In view of the above, when the cam 564 is in contact with the deformable tube 566, the first crush valve assembly 540 is closed, and when the cam 564 not in contact with the deformable tube 546, the second crush valve assembly 560 is open.

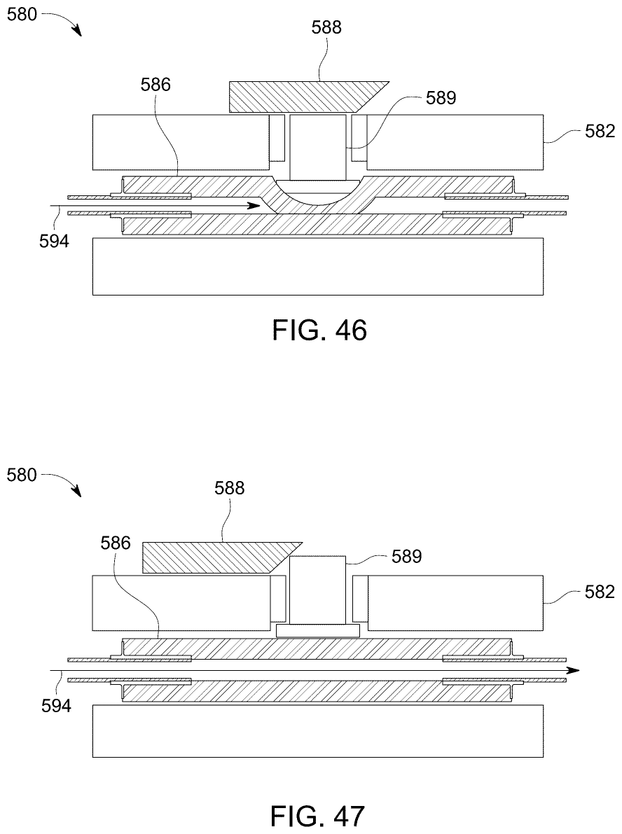

FIG. 46 is a cross-section of a third crush valve assembly 580 in a closed position, and FIG. 47 is a cross-section of the third crush valve assembly 580 in an open position. FIGS. 46-47 are discussed below concurrently. In some examples, the first axial tube valve assembly 154 (or the first axial tube valve assembly 354) may be replaced by the third crush valve assembly 580. The third crush valve assembly 580 includes a deformable tube 586 which deforms to enable or disable a flow 594 of gas to a probe assembly, such as the probe assembly 110 or 310, from a proximal supply line to a distal supply line.

The third crush valve assembly 580 may be similar to the first crush valve assembly 540 previously described above, except in that the refrigerant controller 588 may translate, in a direction parallel to the longitudinal axis A1, to cause a ramp 592 thereof to engage a crush member 589. As may be appreciated, because the ramp 592 slidably engages the crush member 589, the crush member 589 will translate in a direction perpendicular to the longitudinal axis A1 and impart a crush force to the deformable tube 586. In view of the above, when the crush member 589 is in contact with the deformable tube 566, the third crush valve assembly 580 is closed, and when the crush member 589 is not in contact with the deformable tube 586, the third crush valve assembly 580 is open.

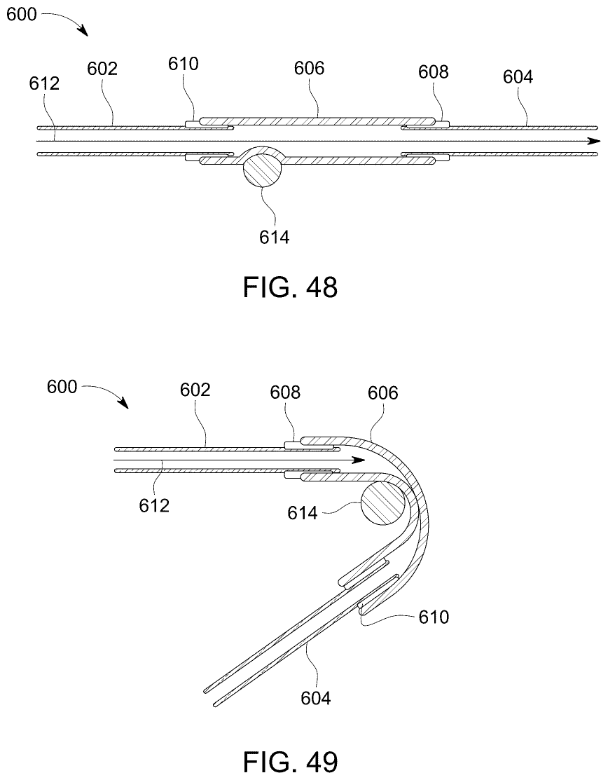

FIG. 48 is a cross-section of a fourth crush valve assembly 600 in an open position, and FIG. 49 is a cross-section of the fourth crush valve assembly 600 in a closed position. FIGS. 48-49 are discussed below concurrently. In some examples, the first axial tube valve assembly 154 (or the first axial tube valve assembly 354) may be replaced by the fourth crush valve assembly 600. The fourth crush valve assembly 600 may be similar to the first crush valve assembly 540, at least in that the fourth crush valve assembly 600 includes a deformable tube 606 connected to a proximal supply line 602 and a distal supply line 604, and which deforms to enable or disable a flow 612 of gas to a probe assembly, such as the probe assembly 110 or 310.

The proximal supply line 602 may be secured to the deformable tube 606 via a first coupling member 610 and the distal supply line 604 may be secured to the deformable tube 606 via a second coupling member 608. In various examples, the first coupling member 610 and the second coupling member 608 may be representative of adhesives, polymeric or elastomeric connectors, or metallic connectors, among others.

In the examples of FIGS. 48-49, a refrigerant controller of a sampling system, such as the sampling systems 106 or 306, may either pivot the proximal supply line 602 of the distal supply line 604 to cause the deformable tube 606 to bend around a fixed member 614 and kink the deformable tube 606. In view of the above, when the deformable tube 606 is wrapped around the fixed member 614, the fourth crush valve assembly 600 is closed, and when the deformable tube 606 is not wrapped around the fixed member 614, the fourth crush valve assembly 600 is open.

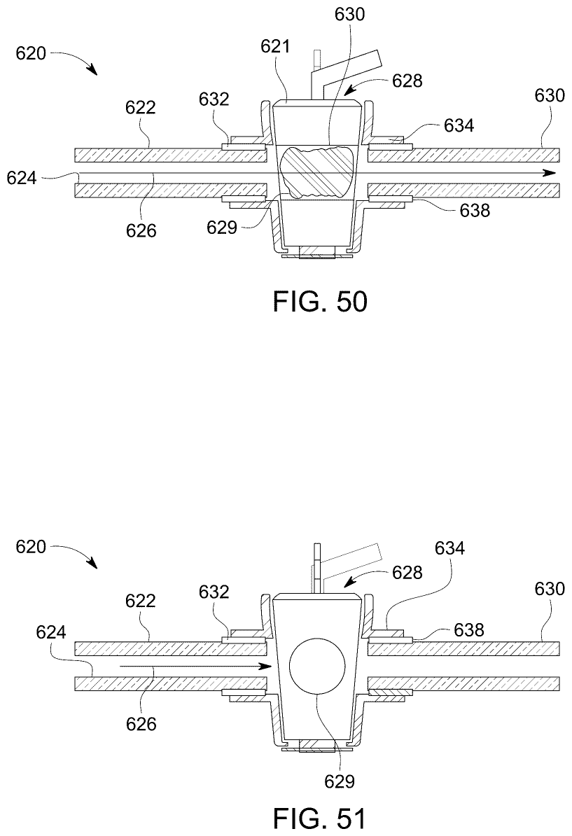

FIG. 50 is a cross-section of a rotatable valve assembly 620 in an open position, and FIG. 51 is a cross-section of the rotatable valve assembly 620 in a closed position. FIGS. 50-51 are discussed below concurrently. In some examples, the first axial tube valve assembly 154 (or the first axial tube valve assembly 354) may be replaced by the rotatable valve assembly 620. The rotatable valve assembly 620 includes a valve body 634 housing a refrigerant controller 628 which rotates in a direction perpendicular to the longitudinal axis A1 of the sampling system 106 (or the sampling system 306) and within the valve body 634 to enable or disable a flow 626 of gas to a probe assembly, such as the probe assembly 110 or 310, through an aperture or opening 629 in the refrigerant controller 628. The valve body 634 is coupled to a proximal supply line 622 for supplying pressurized gas, such as the proximal supply line 140, and a distal supply line 630 for sending pressurized gas to a filter and distal supply line, such as the filter 160 and the distal supply line 161.

The proximal supply line 622 may be secured to the valve body 634 via a first coupling member 632 and the distal supply line 630 may be secured to the valve body 634 via a second coupling member 638. In various examples, the first coupling member 632 and the second coupling member 638 may be representative of adhesives, polymeric or elastomeric connectors, or metallic connectors, among others. In view of the above, when the refrigerant controller 628 is an axially aligned orientation, such as shown in FIG. 50, the rotatable valve assembly 620 is open, and when the refrigerant controller 628 is a non-aligned orientation, such as the perpendicular position shown in FIG. 51, the rotatable valve assembly 620 is closed.

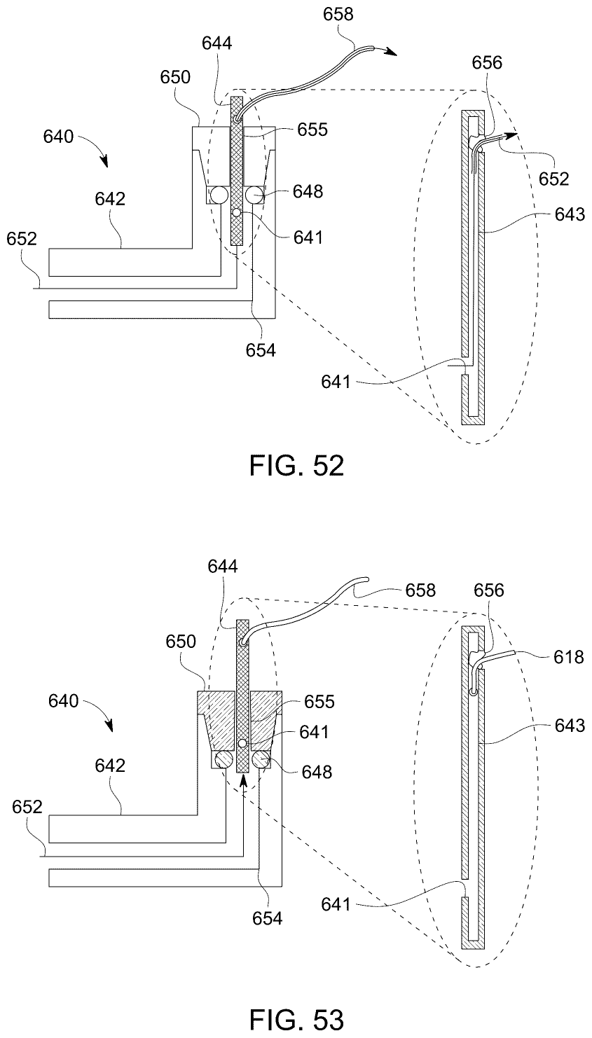

FIG. 52 is a cross-section of a third vertical tube valve assembly 640, and FIG. 53 is a cross-section of the third vertical tube valve assembly 640. FIGS. 52-53 are discussed below concurrently. The third vertical tube valve assembly 640 includes a tubular element 644 which, unlike the first axial tube valve assembly 154, translates orthogonally to the longitudinal axis A1 of the sampling system 106 (or the sampling system 306) to enable or disable a flow 652 of compressed gas to a distal supply line of a probe assembly, such as, but not limited to, the distal supply line 161 previously described above.

The third vertical tube valve assembly 640 may include a first seal 648 and an end cap 650 received by a valve body 642. The end cap 650 may include a first passage 655 for guiding the tubular element 644, and the valve body 642 may include a second passage 654 for guiding the flow 652 of gas, which may function as, or may be connected to, a proximal supply line such as the proximal supply line 140. The first seal 648 is positioned within the valve body 642 to ensure gas may escape the valve body 642 only by passing through a passage 643 in the tubular element 644. As such, when an aperture 641 in a sidewall of the tubular element 644 is beneath the first seal 648, the third vertical tube valve assembly 640 is open, and when the aperture 641 is above the first seal 648, the third vertical tube valve assembly 640 is closed.

Additionally, in the example of FIGS. 52-53, the tubular element 644 may include a second aperture 656 sealed by, for example, an adhesive or other sealing material through which a flexible distal supply line 658 passes. Thus, the third vertical tube valve assembly 640 does not require a second seal like the first vertical tube valve assembly and the second vertical tube valve assembly. It is also appreciated that such an arrangement (e.g., a second aperture in the tubular element having a sealed distal supply line extending thereinto) may also be applied to an axial tube valve to eliminate the need for a seal on both or opposing sides of a first aperture in the tubular element.

For example, the securing member 724 may contact and engage an outer surface or circumference of the first sleeve element 726, and an inner surface or circumference of the outer sheath 312 may be bonded to the outer surface or circumference of the first sleeve element 726. In one specific example, the outer sheath 312 may be bonded to the first sleeve element 726 via the application of heat, such as in an example where the outer sheath 312 comprises a type of heat shrinkable tubing. In some examples, the first sleeve element 726 may be a hypo tube comprised of metallic materials including, but not limited to, stainless steel, titanium, or coated or non-coated steel.

FIG. 55 is a cross-section of a distal tip 816 of a probe assembly 810, according to one example of the present disclosure. The probe assembly 810 may be similar to the probe assembly 110 and 310 previously described above, at least in that the probe assembly 810 may include a distal supply line 861 adapted to function similarly to the distal supply line 161 and 361. The probe assembly 810 may also include a cavity 800 adapted to function similarly to the cavity 200 of the distal tip 116, or the cavity 700 of the distal tip 316, at least by providing additional space or volume for refrigerant exiting the distal supply line 861 to boil or otherwise expand into a gaseous state to enhance or otherwise improve cooling of the distal tip 816. Moreover, the distal supply line 861 may, in some examples, also include the flow regulator 204 to limit or control the refrigerant flow rate through the distal supply line 861 in the distal tip 816.

Additionally, the distal tip 816 or the inner sheath 814 may define similar outer dimensions to the distal tip 116 and the inner sheath 114, or the distal tip 316 and the inner sheath 314 and/or thermal jacket 315 previously described above. In one specific example, the distal tip 816 and the inner sheath 814 may each define an outer diameter of about 0.95 millimeters. In another specific example, the distal tip 816 and the inner sheath 814 may each define an outer diameter of about 1.1 millimeters. Further, the distal tip 816 may form any of the various shapes, or include any of the various additional features, such as radiopaque markings or textures, or a radiopaque coating, previously described above with respect to the distal tip 116 and the distal tip 316.

However, in contrast to the probe assembly 110 and the probe assembly 310 previously described above, the probe assembly 810 may further include a bore 801 defined through a distal end surface 811 of the distal tip 816 and extending coaxially with the distal supply line 861 and/or the cavity 800. The bore 801 may also extend proximally within the inner sheath 314 into a sampling system which includes the probe assembly 810. Such a sampling assembly may be similar to and/or may include any of various features of, the sampling system 106 or 306, except in that the bore 801 may fluidly connected to a splitter, plenum, or other feature or mechanism therein adapted to establish fluid communication between a valve assembly of the sampling system and a proximal terminus of the bore 801.

In some such examples, the splitter, plenum, or other feature or mechanism may fluidly connect a first valve assembly (e.g., the first axial tube valve assembly 154 or 354, or the valve assemblies shown in, and described with reference to, FIGS. 33-53 above) of the sampling system to the bore 801 to thereby cause the bore 801 to receive refrigerant when the distal supply line 861 receives refrigerant, such as when the refrigerant controller 120 or 320 previously described above is positioned in a fully open or partially open position. In other examples, the bore 801 may alternatively be fluidly connected to a second valve assembly (e.g., the first axial tube valve assembly 154 or 354, the valve assemblies shown in, and described with reference to, FIGS. 33-53 above, among others) of the sampling system that is separate and independent from the first valve assembly. In such examples, the second valve assembly may be adapted to control the flow of refrigerant from a proximal supply line, such as the proximal supply line 140 or 340, into the bore 801.

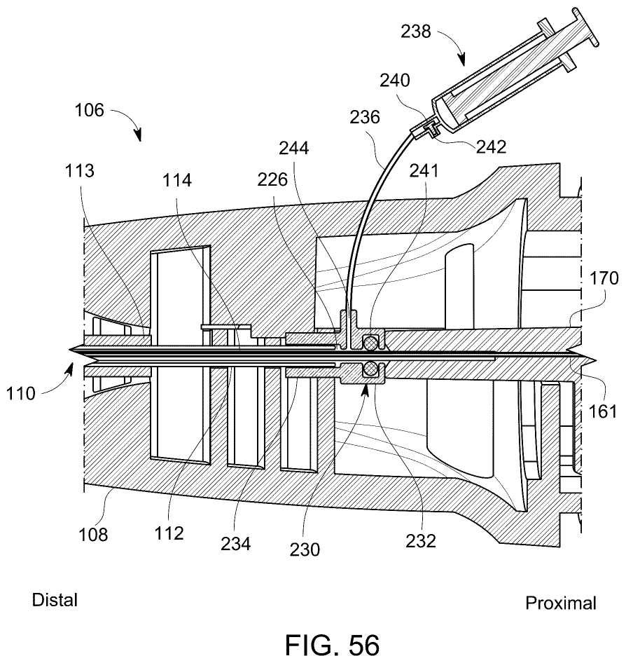

In some examples, a proximal end or terminus of the bore 801 located within a sampling system may be fluidly connected to a suction source, such as a locking or non-locking syringe, located externally to the sampling system. In one such example, the suction source may be the syringe 238 illustrated in FIG. 56. In such an example, the bore 801 may be used to retrieve a tissue sample via aspiration in a manner similar to an aspiration biopsy. The bore 801 may also define various diameters, such as, but not limited to, a diameter measuring within an inclusive range of about 0.015 inches to about 0.050 inches. In one example, the bore 801 may define a diameter of about 0.028 inches.

In an example method of performing a surgical procedure using a cryobiopsy device including the probe assembly 810, a user may, after first connecting a sampling system from which the probe assembly 810 extends to the refrigerant system 104, such as previously discussed above with respect to the sampling system 106 and the sampling system 306, guide an endoscope with the probe assembly 810 positioned therein to a target anatomical location. In some examples, the user may then advance an outer sheath containing the distal tip 816 and the inner sheath 814, such as an outer sheath similar to the outer sheath 112 or the inner sheath 114 previously described above, beyond a distal end of the endoscope to a location adjacent tissue to be biopsied, ablated, or otherwise treated. Next, the user may translate the distal tip 816 out of the outer sheath and into contact with tissue.

In some examples, a user may then activate a suction source in fluid communication with the bore 801 to thereby extract a tissue sample from the patient through the bore 801 in a manner similar to an aspiration biopsy. Alternatively, if a larger tissue sample is desired, the user may instead open a valve assembly of the sampling system to allow refrigerant from the refrigerant system 104 to begin cooling the distal tip 816 and thereby cause tissue to adhere around an outer surface or area the distal tip 816. In either of the above examples, if tissue bleeding is observed by the user, such as after the distal tip 816 pierces tissue and a tissue sample is aspirated through the bore 801 or pulled proximally after cryo-adhesion to the distal tip 816, a user may open a valve assembly within the sampling system to allow refrigerant from the refrigerant system 104 to flow through the bore 801 and freeze and/or ablate tissue nearby tissue to inhibit or stop any observable bleeding.

In further examples, rather than collecting a biopsy sample, the user may instead translate the distal tip 816 outwardly from an outer sheath and open a valve assembly of the sampling system to cause refrigerant from the refrigerant system 104 to flow through the bore 801 and distally beyond the distal end surface 811 to ablate targeted tissue in order to treat various medical conditions through cryo-ablation. Alternatively, or additionally, in some examples, the flow of refrigerant through the bore 801 may be used to cool and/or freeze tissue to help treat Leukoplakia, lung transplant anastomotic strictures, tracheal stenosis, malignancies and/or tumors, respiratory papillomatosis, benign strictures, post-intubation tissue trauma, hemoptysis, hypoxic pulmonary vasoconstriction (“HPV”), Wegener′ granulomatosis, among other medical conditions, or in stent or stricture management.

Further, one of skill in the art will, in light of this teaching, understand that cryoablation procedures, where a cooling effect from a hollow distal tip may be used to destroy abnormal tissues (palliative devitalization), such as to treat, among other conditions, cancer (e.g., kidney, liver, lung, or prostate tumors), heart disorders (e.g., atrial fibrillation or other arrhythmias), or skin conditions (e.g., warts or precancerous lesions). Subsequently, in the above example method, the probe assembly 810 may be retracted proximally into a working channel of the endoscope 302 and the endoscope may then be withdrawn from the patient.

FIG. 56 is a cross-section of the sampling system 106 including a suction connector 230, according to one example of the present disclosure. In some examples, the sampling system 106 may be further adapted to allow an operating physician or clinician to determine whether a blood vessel has been pierced during a surgical procedure. In some such examples, as illustrated in FIG. 56, a gap 226 defined between an inner diameter of the outer sheath 112 and an outer diameter of the inner sheath 114 may be configured (e.g., sized) to allow for aspiration of blood or other fluids therethrough. In other examples, such as illustrated in FIG. 18, the outer sheath 112 may instead define a lumen 228 extending longitudinally therethrough that is separate from a lumen in which the inner sheath 114 is translatably received.

In some examples, the sampling system 106 may include a suction connector 230. The suction connector 230 may be a component or a structure adapted to facilitate suction or aspiration through the gap 226, or alternately, through the lumen 228 (FIG. 18) defined the outer sheath 112. For example, the suction connector 230 may include a distal portion 234 that is bonded, laser welded, adhered, or otherwise sealed to an outer diameter or surface of the outer sheath 112, and a proximal portion 232 in fluid communication with a syringe 238, or another type or style of suction source, via a suction line 236 passing through the outer housing 108 and into a suction passage 244 of the proximal portion 232. Additionally, the proximal portion 232 may also be adapted to retain a seal element 241 that is sized and shaped to seal around an outer circumference of the inner sheath 114 to establish an air-tight seal therebetween. The seal element 241 may, in some examples, be made from similar materials as the first seal 156 or the second seal 157 previously discussed above.

In some examples, the suction line 236 may be detachably connected to the syringe 238 via a luer-lock or other types of connecting mechanisms known in the art. In some examples, such a connecting mechanism may include a stopcock 240 to help provide for precise control over fluid withdrawal, and/or connect the suction line 236 to a waste line or container at an exit port 242 so that aspirated fluid does not enter the syringe 238. Finally, it is appreciated that the relative sizes and/or shapes of the shaping of the fluid connector 170, the suction connector 230, and/or the outer sheath 112 may be selected to ensure sufficient axial clearance is present between the fluid connector 170 and the suction connector 230 within the outer housing 108 to enable the sheath 114 and the fluid connector 170 to translate proximally and distally along the longitudinal axis A1 without contacting or otherwise being inhibited by the stationary outer sheath 112 and the suction connector 230.

In the operation of any of the above examples, a user may, such after the user has advanced the distal tip 116, 316, and/or 816 into contact with tissue, apply suction to the probe assembly 110 using the syringe 238 to determine whether a blood vessel has been pierced or damaged. For example, when the distal tip 116, 316, and/or 816 has entered tissue, the user may open the stopcock 240 and operate the syringe 238 to cause fluid to be drawn through the gap 226 or the lumen 228, through the suction connector 230, and into the suction line 236.

Subsequently, if the fluid is blood, or otherwise contains a significant quantity of blood, the user may decide to move the distal tip 116, 316, and/or the distal tip 816 into a different position, after which the syringe 238 may again be operated to once again determine whether the selected location is suitable for collecting tissue (e.g., if little or no blood is present in the fluid withdrawn through the suction line 236). In view of the above, the suction connector 230, along with the gap 226 and/or the lumen 228, may be advantageous in helping a user to position the probe assembly 110 within a patient. Finally, while the concept of applying suction to monitor for vessel bleeding is discussed above with respect to the sampling system 106, it is also appreciated that the sampling system 306 may be modified in a similar fashion. However, in this regard, the inventors have also appreciated that monitoring tissue bleeding via aspiration may not be necessary in surgical procedures involving ultrasonic imaging endoscopes, such as the endoscope 302, as blood vessels may instead be monitored through ultrasonic imaging and thus the devices or systems used therewith (e.g., the sampling system 306) may not need a suction source and suction gaps and/or lumens.

Although not specific discussed above, one of skill in the art will, in light of this teaching, understand that the biopsy systems described above may be applied to, among others, pulmonary biopsies (e.g., biopsies of the lungs and/or respiratory system), urology biopsies, (e.g., biopsies of the bladder, ureter, or renal tissue), gastrointestinal biopsies (e.g., biopsies of the bile duct or pancreatic duct), thoracic biopsies (e.g., biopsies of the pleural space tissue), abdominal biopsies (e.g., biopsies of the peritoneum or solid organ metastases), dermatologic biopsies (e.g., biopsies of skin), among others, or in submucosal and extramural lesion removal of the tracheobronchial tree, among many other types and locations of internal or external lesions.

Finally, although the invention has been described above in terms of particular embodiments and applications, one of ordinary skill in the art will, in light of this teaching, be able to generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A cryobiopsy device, comprising:
a refrigerant system including a refrigerant cartridge; and
a sampling system including:
a distal tip translatable relative to an outer sheath;
a position controller connected to the distal tip; and,
a gas controller.

2. The cryobiopsy device of claim 1, wherein the position controller is translatable between at least a first position in which the distal tip is within an outer sheath and a second position in which the distal tip extends distally beyond the outer sheath; and, wherein the sampling system further includes a refrigerant controller movable between an open position in which refrigerant may flow through the sampling system to the distal tip and a closed position in which refrigerant is prevented from flowing through the sampling system to the distal tip.

3. The cryobiopsy device of claim 2, wherein the refrigerant system further comprises:
a cartridge nest holding the refrigerant cartridge; and
a base housing receiving the cartridge nest and fluidly connecting the refrigerant cartridge to sampling system.

4. The cryobiopsy device of claim 3, wherein the base housing defines a first plurality of threads and the cartridge nest defines a second plurality of threads engaged with the first plurality of threads.

5. The cryobiopsy device of claim 4, wherein the base housing includes a piercing element passing through a septum of a refrigerant cartridge within the cartridge nest.

6. The cryobiopsy device of claim 3, further comprising a valve assembly in fluid communication with the refrigerant system, the valve assembly movable between an open position in which gas flows through the valve assembly to the distal tip and a closed position in which refrigerant is prevented from flowing through the valve assembly to the distal tip.

7. The cryobiopsy device of claim 6, further comprising:
a proximal supply line fluidly connecting the refrigerant cartridge to the valve assembly; and
a tube encompassing the proximal supply line between an outer housing of the sampling system and the base housing of the refrigerant system, the tube fluidly connecting an exhaust port in fluid communication with a whistle chamber of the base housing to an exhaust line located within the outer housing.

8. The cryobiopsy device of claim 6, wherein the sampling system includes a probe assembly extending distally from an outer housing, the probe assembly comprising:
an outer sheath connected to the outer housing; and
an inner sheath connected to the position controller, wherein the distal tip is received within, and extends distally beyond, a distal end of the inner sheath.

9. The cryobiopsy device of claim 8, wherein the inner sheath includes a wire positioned to regulate refrigerant flow to the distal tip within the inner sheath.

10. The cryobiopsy device of claim 8, wherein the valve assembly includes a translatable tubular element which opens and closes the valve assembly.

11. The cryobiopsy device of claim 10, wherein an aperture extends through a sidewall of the tubular element, wherein the aperture is aligned with a proximal supply line when the valve assembly is open.

12. The cryobiopsy device of claim 11, wherein the refrigerant controller is translatable in a direction parallel to a longitudinal axis to open and close the valve assembly.

13. The cryobiopsy device of claim 11, wherein the refrigerant controller is engageable with the position controller to lock the valve assembly in an open position.

14. (canceled)

15. The cryobiopsy device of claim 11, wherein the position controller is configured to guide the distal tip between, and maintain the distal tip in, a retracted position, a partially extended position, and a fully extended position.

16. A cryobiopsy device, comprising:
a refrigerant system including a refrigerant cartridge; and
a sampling system including:
a scope adapter including a connection mechanism;
an outer housing translatable between at least a first position relative to the scope adapter in which a distal tip is within an outer sheath and a second position relative to the scope adapter in which the distal tip extends distally beyond the outer sheath; and
a refrigerant controller.

17. The cryobiopsy device of claim 16, further comprising a central body connecting the outer housing to the scope adapter, wherein the outer housing is translatable about the central body to extend and retract the distal tip.

18. The cryobiopsy device of claim 17, further comprising a pawl assembly, wherein the central body includes a plurality of teeth engageable by the pawl assembly to prevent translation of the outer housing about the central body.

19. The cryobiopsy device of claim 18, wherein the central body is adjustably connected to the scope adapter to adjust a position of a distal end of the cryobiopsy device with respect to a distal end of an endoscope.

20. The cryobiopsy device of claim 16, further comprising a valve assembly located within the outer housing and in fluid communication with the refrigerant system, the valve assembly movable between an open position in which refrigerant flows through the valve assembly to the distal tip and a closed position in which refrigerant is prevented from flowing through the valve assembly to the distal tip.

21. (canceled)

22. The cryobiopsy device of claim 20, wherein the valve assembly includes a tubular element translatable in a direction parallel to a longitudinal axis of the sampling system to open or close the valve assembly.

23-35. (canceled)
